(19) United States
(12) Patent Application Publication
(10) Pub. No.: US 2026/0018244 A1
(43) Pub. Date: Jan. 15, 2026

(54) METHODS AND SYSTEMS FOR PREDICTION OF PEPTIDE PRESENTATION BY MAJOR HISTOCOMPATIBILITY COMPLEX MOLECULES

(71) Applicant: Genentech, Inc., South San Francisco, CA (US)

(72) Inventors: Suchit Sushil JHUNJHUNWALA, Foster City, CA (US); Kai LIU, Belmont, CA (US); Nicolas Winston LOUNSBURY, Redwood City, CA (US); Jason PERERA, Chicago, IL (US); William John THRIFT, San Mateo, CA (US); Adric Quade BROADWELL, San Francisco, CA (US); Jieming CHEN, Foster City, CA (US)

(21) Appl. No.: 19/228,568

(22) Filed: Jun. 4, 2025

Related U.S. Application Data
(63) Continuation of application No. PCT/US2023/082356, filed on Dec. 4, 2023.
(60) Provisional application No. 63/430,297, filed on Dec. 5, 2022.

(51) Int. Cl. G16B 15/30; G16B 40/20; G16H 20/10

(52) U.S. Cl. CPC G16B 15/30; G16B 40/20; G16H 20/10

(57) ABSTRACT

This present disclosure relates to immunology, particularly methods of predicting whether a therapeutic protein is likely to trigger an immunogenic response. An example method for predicting an amino acid-immunoprotein complex (IPC) interaction may comprise: accessing a set of amino acid sequences; accessing an immunoprotein complex (IPC) sequence identified for an IPC of a subject; processing a set of amino acid sequence representations to generate a set of transformed amino acid sequence representations based on a set of element-focused scores representing binding cores of the set of amino acid sequence representations; processing an IPC sequence representation to generate a transformed IPC sequence representation; generating composite representations; and determining one or more predicted amino acid-IPC interactions based on the composite representations.

### CROSS-REFERENCE TO RELATED APPLICATIONS

This application is a continuation of International Application No. PCT/US2023/082356, filed on Dec. 4, 2023, which claims priority to U.S. Provisional Patent Application No. 63/430,297, filed on Dec. 5, 2022, entitled “PREDICTION OF PEPTIDE PRESENTATION BY MAJOR HISTOCOMPATIBILITY COMPLEX MOLECULES,” the content of which is hereby incorporated by reference in its entirety.

### REFERENCE TO AN ELECTRONIC SEQUENCE LISTING

The content of the electronic sequence listing (146392060901seqlist.xml; Size: 70,386 bytes; and Date of Creation: Sep. 26, 2025) is herein incorporated by reference in its entirety.

### FIELD

This present disclosure generally relates to immunology, particularly methods of predicting whether a neoantigen or therapeutic protein is likely to trigger an immunogenic response.

### BACKGROUND

Therapeutic proteins are a type of medicinal product (biologic) obtained from living sources (e.g., animal, plant, fungal, or microbial cells). Many therapeutic proteins, including monoclonal antibodies and soluble receptors, are now produced using recombinant DNA technology. This is in contrast to small molecule drugs, which are typically simpler compounds manufactured by chemical synthesis.

Some therapeutic proteins have the same primary amino acid sequence as native human proteins, which typically does not trigger an immune response. However, it is often desirable to modify the amino acid sequence of a therapeutic protein to optimize various properties such as potency, stability, and bioavailability. However, therapeutic proteins having significant differences in amino acid sequence than proteins of an intended recipient (e.g., human subject) can be recognized as foreign by the recipient's immune system, thereby triggering an immune response as an antigen (a toxin or foreign substance) might do. In this way, a foreign-looking therapeutic protein is perceived to be more of a vaccine than a medicinal compound. While it is imperative for a vaccine to elicit an immune response to be effective, it is detrimental for a therapeutic protein to elicit an immune response. If a therapeutic protein elicits an immune response, it will render the therapeutic protein ineffective and/or will cause a deleterious immune response in the recipient. In particular, repeated administration of an immunogenic therapeutic protein typically elicits anti-drug antibodies (ADA). At best, the immunogenic therapeutic protein will be neutralized by ADA of a recipient, thereby reducing its pharmaceutical activity. At worst, an immunogenic therapeutic protein will induce a hypersensitivity reaction in the recipient, which can be life threatening.

Human leukocyte antigens (HLA) are expressed as cell surface receptors that present antigenic peptides to T cells in a restricted manner, which allows discrimination between self- and foreign antigens. The HLA complex is a complex of genes on chromosome 6 that encodes the cell-surface proteins responsible for the regulation of the immune system. The human major histocompatibility complex (MHC) is residence to HLA genes that play a fundamental role in the acceptance of transplanted tissues. The MHC contains many of the genes associated with cell-mediated immune defenses. The MHC complex encodes the α-chains of the MHC class I molecules HLA-A, HLA-B, and HLA-C (alleles) and the α- and β-chains of the MHC class II molecules HLA-DR, HLA-DP, and HLA-DQ (allotypes), all of which are expressed in a co-dominant fashion.

Activation of helper T (Th) cells upon recognition of peptide fragments (epitopes) of a protein antigen, which are bound to MHC class II (MHC-II) molecules of antigen-presenting cells, results in the development of antigen-reactive antibodies. If the antigen is a therapeutic protein, the antibodies will be ADA. Peptide binding to an MHC molecule at a sufficient affinity is a prerequisite for immunogenicity (ability of a peptide to trigger an immune response). As such, it would be desirable to predict MHC-II epitopes of a candidate therapeutic protein so as to identify amino acid residues of the protein that could be safely altered, thereby eliminating MHC-II epitopes of the candidate therapeutic protein, in order to reduce its immunogenicity. Peptide-MHC binding affinity is primarily determined by the amino acid sequence of the peptide binding core (typically nine amino acids long); however, the amino-terminal flanking (N-flank) sequence and/or the carboxy-terminal flanking (C-flank) sequence may also affect peptide-MHC binding.

Tumors, like the subjects they afflict, are heterogeneous. In particular, the somatic mutations that cause a cell to become cancerous vary even among tumors derived from the same cell type. Moreover, while humans are predicted to share 99.9% of their genome, the 0.1% difference is consequential, especially in terms of the immune system. As such, a therapeutic cancer vaccine is ideally designed as a personalized cancer vaccine.

Neoantigens are tumor-specific antigens that result from somatic mutations in a tumor cell's genome. Peptide fragments (epitopes) of a protein neoantigen bind to major histocompatibility complex (MHC) molecules expressed on the surface of a subject's cancer cells and antigen presenting cells, where they are able to activate CD8+ cytotoxic T lymphocytes (CTL) and CD4+ helper T (Th) cells, respectively. Neoantigen vaccines are a promising approach for individualized cancer therapy in that they are able to prime a subject's T cells to recognize and attack cancer cells expressing neoantigen(s), while sparing healthy cells.

The tumor profile of a subject can be defined by determining DNA and/or RNA sequences from tumor cells obtained from a biopsy. From the subject-specific tumor profile, neoantigens of interest that are present in tumor cells, but absent in healthy cells, can be identified. However, the vast majority of mutant sequences that are detected in tumor cells correspond to neoantigens that are poorly-expressed, do not contain epitopes that are presented by MHC molecules, and/or are otherwise are not bound by T cell receptors (TCRs). Such neoantigens would fail to trigger an immunological response by a CD8+ CTL in the case of MHC class I (MHC-I) molecules, or by a CD4+Th cell in the case of MHC class II (MHC-II) molecules. Consequently, such neoantigens are poor candidates for inclusion in an individualized cancer vaccine for generating a tumor-specific immune response.

There are tools for predicting peptide binding to MHC molecules. However, simply identifying peptide fragments of a neoantigen capable of binding to MHC molecules is insufficient for identifying neoantigens for inclusion in a personalized cancer vaccine. This is because many of the peptide binders will be false positives in that they will not effectively prime a cellular immune response. Thus, what is needed in the art are tools for accurately identifying epitopes of neoantigens that are presented on the surface of tumor such that they provoke a robust immune response to aid in selecting peptide fragments of neoantigens for inclusion in therapeutic cancer vaccines.

### SUMMARY

Disclosed herein are systems, methods, and programming for determining a prediction of whether and/or an extent to which a peptide interacts with an MHC molecule using a machine-learning model. The machine-learning model can perform a combination of peptide data processing, nFlank/cFlank data processing, MHC data processing, TCR data processing, and/or protein data processing for obtaining one or more interaction predictions (e.g., a predicted peptide interaction with an MHC molecule), one or more interaction affinity predictions (e.g., a predicted binding affinity between a peptide and an MHC), and/or one or more immunogenicity predictions (a prediction of the ability of a peptide to provoke an immune response with respect to an MHC) as described herein. In an example workflow, the processing of MHC data can involve processing of a BOS token-appended MHC sequence using one or more transformer stages or alternatively involve processing of an MHC sequence embedding generated using a protein language model. In the example workflow, the processing of peptide data can involve processing of a BOS token-appended peptide sequence using one or more transformer stages or alternatively involve processing of a peptide sequence not appended with a BOS token using a cross-attention module. The example workflow may additionally incorporate the processing of protein data or alternatively may not incorporate the processing protein data. The example workflow may additionally incorporate the processing of TCR data or alternatively may not incorporate TCR data.

In some embodiments, the machine-learning model processes a set of amino acid sequence representations and an immunoprotein complex (IPC) sequence representation using separate processing blocks and in parallel. A sequence representation can be an embedding of features of the corresponding sequence. The machine-learning model uses a set of element-focused scores that represent the binding cores of the set of amino acid sequence representations and combines the BOS token embeddings of transformed amino acid sequence representations with the BOS token embedding of a transformed IPC sequence representation to generate composite representations. The composite representations are used to determine one or more predicted amino acid-IPC interactions, such as an interaction affinity prediction that predicts a binding affinity between a peptide and an MHC, an interaction prediction that predicts whether an MHC will present a peptide at a cell surface, or an immunogenicity prediction that predicts the ability of a peptide to provoke an immune response with respect to an MHC.

Some aspects include accessing a set of amino acid sequences. Each of the amino acid sequences of the set may have been identified from at least one protein. An immunoprotein complex (IPC) sequence identified for an IPC of a subject can be assessed. One or more first processing blocks in a processing subsystem of a machine-learning model can be used to process a set of amino acid sequence representations to generate a set of transformed amino acid sequence representations based on a set of element-focused scores representing binding cores of the set of amino acid sequence representations. Each of the amino acid sequence representations may have been generated based on one of the amino acid sequences appended with a beginning-of-sequence (BOS) token. A second processing block in the processing subsystem can be used to process an IPC sequence representation to generate a transformed IPC sequence representation. The IPC sequence representation may have been generated based on the identified IPC sequence appended with a BOS token. The set of amino acid sequence representations and the IPC sequence representation can be processed in parallel. The system may generate composite representations by combining each of the transformed BOS token representations of the set of transformed amino acid sequence representations with the transformed BOS token representation of the transformed IPC sequence representation. The system may determine one or more predicted amino acid-IPC interactions based on the composite representations.

In some embodiments, the set of transformed amino acid sequence representations comprises a set of amino-terminal flanking (N-flank) representations or a set of carboxy-terminal flanking (C-flank) representations.

In some embodiments, the IPC of the subject is a major histocompatibility complex (MHC).

In some embodiments, the set of transformed amino acid sequence representations comprises a set of MHC-binding representations.

In some embodiments, the MHC comprises MHC class II (MHC-II).

In some embodiments, the MHC comprises MHC class I (MHC-I).

In some embodiments, the IPC of the subject is a T-cell receptor (TCR).

In some embodiments, the at least one protein can be a therapeutic protein.

In some embodiments, the at least one protein is present in a disease sample from the subject.

In some embodiments, the disease sample can be a tumor cell biopsy. In some embodiments, the disease sample includes cancer. In some embodiments, the disease sample includes tissue.

In some embodiments, generating composite representations comprises: for each of the set of transformed amino acid sequence representations, elementwise multiplying a transformed amino acid beginning-of-sequence (BOS) representation corresponding to the transformed amino acid sequence representation by a transformed IPC beginning-of-sequence (BOS) representation corresponding to the transformed IPC sequence representation.

In some embodiments, processing a set of amino acid sequence representations comprises processing a peptide beginning-of-sequence (BOS) representation to generate a transformed peptide sequence representation.

In some embodiments, processing an IPC sequence representation comprises processing an MHC beginning-of-sequence representation (BOS) to generate a transformed MHC sequence representation.

Some additional aspects include, for each of a set of IPC sequences, perform the steps of: accessing the IPC sequence, processing the set of amino acid sequence representations, processing the IPC sequence representation using the respective IPC sequence, and generating the composite representations. The determined one or more predicted amino acid-IPC interactions can be based on the composite representations corresponding to the set of IPC sequences.

In some embodiments, the set of IPC sequences comprises twelve major histocompatibility complex (MHC) allotypes of the subject and/or six MHC alleles of the subject.

Some additional aspects include, selecting one or more peptide-IPC combinations from the set of amino acid sequences and the set of IPC sequences to include as a target for an immunotherapy based on the one or more determined amino-acid IPC interactions.

In some embodiments, processing the set of amino acid sequence representations comprises: transforming the set of amino acid sequence representations using the one or more first processing blocks into the set of transformed amino acid sequence representations. Each of the one or more first processing blocks includes a set of processing sub-blocks.

Some additional aspects include embedding the set of amino acid sequences to generate a set of embedded amino acid sequence representations; and positionally encoding the set of embedded amino acid sequence representations.

In some embodiments, processing the IPC sequence representation comprises: transforming the IPC sequence representation using the second processing block into a transformed IPC sequence representation. The second processing block includes a set of processing sub-blocks.

Some additional aspects include embedding the IPC sequence to generate an embedded IPC sequence representation; and positionally encoding the embedded IPC sequence representation.

In some embodiments, the machine-learning model includes one or more transformer encoders, each of the one or more transformer encoders including a processing layer.

In some embodiments, each of the one or more first processing blocks or the second processing block comprises a set of processing sub-blocks; and each of the set of processing sub-blocks includes a neural network comprising at least one processing layer.

In some embodiments, the set of amino acid sequence representations comprises aggregate sequence representations, the aggregate sequence representations including a set of peptide representations and one or more of: a set of amino-terminal flanking (N-flank) representations or a set of carboxy-terminal flanking (C-flank) representations.

Some additional aspects include, prior to generating the set of transformed amino acid sequence representations: flattening the aggregate sequence representations into a single array; and densifying the aggregate sequence representations by removing empty rows from the array. The transformed amino acid sequence representations are generated based on the densified aggregate sequence representations.

In some embodiments, the IPC sequence representation comprises aggregate sequence representations, the aggregate sequence representations including one or more of: a major histocompatibility complex (MHC) sequence representation or a T-cell receptor (TCR) sequence representation.

In some embodiments, processing the set of amino acid sequence representations comprises: for each amino acid sequence representation of the set: determining, for each element of the amino acid sequence representation, a plurality of vectors based on a set of weights associated with a processing layer of the machine-learning model; and generating the set of element-focused scores based on the plurality of vectors and the set of weights.

In some embodiments, the plurality of vectors comprises a key vector, a value vector, and a query vector, and the set of weights comprises a set of key weights, a set of value weights, and a set of query weights.

In some embodiments, generating the set of element-focused scores comprises: determining each element-focused score from each pair of elements from the query vector and the key vector.

In some embodiments, the one or more first processing blocks and the second processing block comprise attention blocks, each attention block comprising a set of attention sub-blocks, each attention sub-block comprising a self-attention layer; and the machine-learning model is an attention-based machine learning model.

Some additional aspects include by one or more of the attention blocks, generating attention maps including masks limiting attention applied by the attention sub-blocks to a sequence length according to the masks.

Some additional aspects include processing, using a fully connected block in an output subsystem of the machine-learning model, the composition representations to generate a first output; applying, using a dropout block in the output subsystem of the machine-learning model, a dropout to the first output to generate a second output; and selecting, using a max layer in the output subsystem of the machine-learning model, a subset of the second output to generate a result. The one or more predicted amino acid-IPC interactions are determined based on the result.

In some embodiments, the set of amino acid sequences comprises a peptide sequence and the IPC sequence comprises a major histocompatibility complex (MHC) sequence, and the one or more predicted amino acid-IPC interactions comprise one or more of: an interaction affinity prediction for a peptide-IPC combination that predicts a binding affinity between a peptide and MHC; an interaction prediction for the peptide-IPC combination that predicts whether the MHC will present the peptide at a cell surface; or an immunogenicity prediction for the peptide-IPC combination that predicts an ability of the peptide to provoke an immune response with respect to the MHC.

In some embodiments, determining the one or more predicted amino acid-IPC interactions comprises: processing the composite representations to generate a set of results; and selecting an amino acid-IPC combination based on a highest result among the set of results.

In some embodiments, the one or more predicted amino acid-IPC interactions comprise a prediction of tumor-specific immunogenicity of a peptide.

In some embodiments, the set of amino acid sequences comprises a set of peptide sequences. The one or more predicted amino acid-IPC interactions identify a subset of peptide sequences having increased tumor-specific immunogenicity or increased likelihood of presentation by the IPC relative to the set of peptide sequences.

Some additional aspects include identifying a subset of peptides from the set of amino acid sequences to include in an individualized vaccine based on the determined one or more predicted amino acid-IPC interactions.

Some additional aspects include generating a treatment recommendation that includes the individualized vaccine.

Some additional aspects include selecting a subset of peptides from the set of amino acid sequences to include as a target for an immunotherapy based on the determined one or more predicted amino acid-IPC interactions.

In some embodiments, the immunotherapy comprises one or more of: a T cell therapy, a personalized cancer therapy, an antigen-specific immunotherapy, an antigen-dependent immunotherapy, a vaccine, or a natural killer (NK) cell therapy.

Some additional aspects include selecting a subset of peptides from the set of amino acid sequences to exclude as a target for an immunotherapy based on the determined one or more predicted amino acid-IPC interactions.

In some embodiments, the immunotherapy comprises one or more of: a T cell therapy, a personalized cancer therapy, an antigen-specific immunotherapy, an antigen-dependent immunotherapy, a vaccine, or a natural killer (NK) cell therapy.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed can be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 is a block diagram of an example prediction system, in accordance with some embodiments.

FIG. 2 is a flowchart of an example process for predicting an amino acid-immunoprotein complex (IPC) interaction using a machine-learning model, in accordance with some embodiments.

FIG. 3 is a schematic diagram of an example configuration for a machine-learning model, in accordance with some embodiments.

FIG. 4A is an example workflow diagram for obtaining one or more interaction predictions, one or more interaction affinity predictions, and/or one or more immunogenicity predictions, in accordance with some embodiments.

FIG. 4B is an example workflow diagram for obtaining one or more interaction predictions, one or more interaction affinity predictions, and/or one or more immunogenicity predictions, in accordance with some embodiments.

FIG. 4C is an example workflow diagram for obtaining one or more interaction predictions, one or more interaction affinity predictions, and/or one or more immunogenicity predictions, in accordance with some embodiments.

FIG. 4D is an example workflow diagram for obtaining one or more interaction predictions, one or more interaction affinity predictions, and/or one or more immunogenicity predictions, in accordance with some embodiments.

FIG. 4E illustrates an example protein language model for generating a protein sequence embedding, in accordance with some embodiments.

FIG. 4F illustrates an example protein language model for generating an MHC sequence embedding, in accordance with some embodiments.

FIG. 4G is an example workflow diagram for predicting multiple peptide interactions with an MHC molecule that may have been expressed by multiple alleles or allotypes, in accordance with some embodiments.

FIG. 4H is an example workflow diagram for attention masking, in accordance with some embodiments.

FIG. 4I is an example workflow diagram for attention masking with a calibration step for reducing model bias, in accordance with some embodiments. Sequences on x-axis and y-axis (SEQ ID NO: 14).

FIGS. 5A-5C are schematic diagrams of example machine-learning models, in accordance with some embodiments.

FIG. 6 is a schematic diagram of an example processing block, in accordance with some embodiments.

FIG. 7A is a flowchart of an example process for processing a sequence representation using an example processing layer, in accordance with some embodiments.

FIG. 7B is a schematic diagram illustrating an example process for processing a sequence representation using an example processing layer, in accordance with some embodiments.

FIG. 8 is a flowchart of an example process for generating information about immunological activity of various peptides, in accordance with some embodiments.

FIG. 9 is a flowchart of an example process for generating information about immunological activity of various peptides, in accordance with some embodiments.

FIG. 10 is a flowchart of an example process for training a machine-learning model and using the trained machine-learning model to generate predictions relating to amino acids and IPCs, in accordance with some embodiments.

FIG. 11 is an illustration that includes an example of training data, in accordance with some embodiments. In this figure, the IPCs are MHC class II allotypes (HLA-DR, HLA-DP, and HLA-DQ allotypes). nFlank sequences from top to bottom: SEQ ID NO: 15, SEQ ID NO: 18, SEQ ID NO: 21, SEQ ID NO: 24, SEQ ID NO: 27, SEQ ID NO: 30, SEQ ID NO: 32, SEQ ID NO: 34, SEQ ID NO: 36, SEQ ID NO: 39. Peptide sequences from top to bottom: SEQ ID NO: 16, SEQ ID NO: 19, SEQ ID NO: 22, SEQ ID NO: 25, SEQ ID NO: 28, SEQ ID NO: 31, SEQ ID NO: 33, SEQ ID NO: 35, SEQ ID NO: 37, SEQ ID NO: 40. cFlank sequences from top to bottom: SEQ ID NO: 17, SEQ ID NO: 20, SEQ ID NO: 23, SEQ ID NO: 26, SEQ ID NO: 29, SEQ ID NO: 38, SEQ ID NO: 41.

FIG. 12 is an example method for predicting which therapeutic antibodies are likely to increase immunogenicity risk. 1205 sequence (SEQ ID NO: 42). 1210 sequences (SEQ ID NO: 43, SEQ ID NO: 44, SEQ ID NO: 45, SEQ ID NO: 46). 1215 sequences (SEQ ID NO: 47, SEQ ID NO: 48, SEQ ID NO: 49, SEQ ID NO: 50, SEQ ID NO: 51, SEQ ID NO: 52). 1220 sequences (SEQ ID NO: 53, SEQ ID NO: 54, SEQ ID NO: 55, SEQ ID NO: 56, SEQ ID NO: 57, SEQ ID NO: 58). 1225 sequences (SEQ ID NO: 59, SEQ ID NO: 60, SEQ ID NO: 61, SEQ ID NO: 62). 1230 sequences (SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71). 1240 sequences (SEQ ID NO: 72, SEQ ID NO: 73, SEQ ID NO: 74, SEQ ID NO: 75, SEQ ID NO: 76). 1250 sequences (SEQ ID NO: 77, SEQ ID NO: 78).

FIG. 13 is an illustration of an example neoantigen candidate (mutant antigen) and the corresponding potential neoepitope candidates (mutant peptides), in accordance with some embodiments.

FIG. 14A includes an example plot indicating the performance of an example P-MHC-II Model, in accordance with some embodiments.

FIG. 14B includes an example plot indicating the performance of a previously-used approach, Model A, with respect to its elution output, in accordance with some embodiments.

FIG. 15 is an example plot comparing example average precision values of elution-ligand outputs of a previously-used approach, Model A, and an example P-MHC-II Model for each allotype in a test data set, in accordance with some embodiments.

FIGS. 16A and 16B are example plots that illustrate the performance of an example P-MHC-II Model and previously used approach, Model A, respectively, in accordance with some embodiments.

FIG. 17 illustrates a plot of a latent space that includes a plurality of peptide vectors, in accordance with some embodiments.

FIG. 18 illustrates a histogram showing the counts of peptides having different levels of information content in an example dataset, in accordance with some embodiments.

FIG. 19A illustrates a protein space colored by protein expression, in accordance with some embodiments.

FIG. 19B illustrates the cellular compartmentalization of different proteins and where they appeared in the latent space, in accordance with some embodiments.

FIG. 20 illustrates example performance data, in accordance with some embodiments.

FIG. 21 is a block diagram of a computer system, in accordance with some embodiments.

FIG. 22 is a block diagram of an example artificial intelligence (AI) architecture included as part of the example computing system of FIG. 16, in accordance with some embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

Recognizing the importance of being able to predict which mutant peptides (e.g., neoantigens) to select as candidates for an individualized vaccine, the embodiments described herein provide methodologies and systems for making more accurate predictions than currently available methods and systems. The embodiments described herein use machine-learning methodologies and systems to improve prediction performance by, for example, without limitation, reducing the number of false positives generated when analyzing mutant peptide sequences to determine the viability of those mutant peptides as vaccine candidates. The embodiments described herein also provide methodologies and systems for determining whether certain therapeutic antibodies may present an immunogenicity risk to a subject.

Disclosed herein are systems, methods, and programming for obtaining one or more interaction predictions (e.g., a predicted peptide interaction with an MHC molecule), one or more interaction affinity predictions (e.g., a predicted binding affinity between a peptide and an MHC), and/or one or more immunogenicity predictions (a prediction of the ability of a peptide to provoke an immune response with respect to an MHC) as described herein. The machine-learning model can perform a combination of peptide data processing, nFlank/cFlank data processing, MHC data processing, TCR data processing, and/or protein data processing for predicting a peptide interaction with an MHC molecule. In an example workflow, the processing of MHC data can involve processing of a BOS token-appended MHC sequence using one or more transformer stages or alternatively involve processing of an MHC sequence embedding generated using a protein language model. In the example workflow, the processing of peptide data can involve processing of a BOS token-appended peptide sequence using one or more transformer stages or alternatively involve processing of a peptide sequence not appended with a BOS token using a cross-attention module. The example workflow may additionally incorporate the processing of protein data or alternatively may not incorporate the processing protein data. The example workflow may additionally incorporate the processing of TCR data or alternatively may not incorporate TCR data.

For example, the embodiments described herein provide a machine-learning model, various methodologies of using a machine-learning model, and/or the output generated by a machine-learning model to analyze sequences identified from a disease sample from a subject. To predict whether and/or the extent to which a mutant peptide identified from the disease sample interacts with an IPC such as an MHC molecule (e.g., MHC-I or MHC-II), the machine-learning model processes a set of amino acid sequence representations separately from the processing of an IPC sequence representation (e.g., an MHC sequence representation). A sequence representation can be an embedding of features of the corresponding sequence. In some embodiments, a mutant peptide sequence representation can be referred to as a variant-coding sequence. An IPC sequence (e.g., MHC sequence) may comprise at least a portion of an MHC molecule, the full sequence, a pseudosequence of the MHC molecule (the portion that interacts with the mutant peptide (including a binding pocket, some other portion that includes the pseudosequence, etc.)).

The machine-learning model includes various subsystems for processing. The machine-learning model may include, for example, a representation subsystem, a processing subsystem, a composite subsystem, and an output subsystem. Each “subsystem” may comprise one or more blocks, with each block comprising one or more sub-blocks and/or layers. A sub-block may comprise any number of layers (or units).

A processing subsystem can be used to generate one or more transformed sequence representations such as a set of transformed amino acid sequence representations (e.g., which may include a variant-coding sequence), a transformed IPC sequence representation, etc. In some embodiments, the processing subsystem may process one or more (e.g., a set of) amino acid sequence representations independent of, or separately from (e.g., in parallel), an IPC sequence representation. For example, a set of amino acid sequence representations can be processed using one or more first processing blocks in a processing subsystem, and the IPC sequence representation can be processed using a second processing block in the processing subsystem. Processing the set of amino acid sequence representations and the IPC sequence representation via these parallel processing engines and/or separate processing blocks may improve the predictive performance of the machine-learning model. The separate processing engines can force the system to learn separate representations for different biological structures. In contrast, processing different biological structures via a shared processing engine may cause model overfitting.

Further, the embodiments described herein recognize and take into account that training a model corresponding to a series of biological events may require significantly more data than training a model corresponding to a single biological event. Training a model for sequence analysis can be particularly complicated due to the sheer number of sequences potentially observable. Not only are there millions of potential neoantigens, but genes encoding the proteins for MHC class-II molecules, for example, are also highly polymorphic. In fact, nearly 6,000 variant alpha and beta chain proteins of HLA-DR, HLA-DQ and HLA-DP (three classical class-II molecules of humans) are currently present in the IPD-IMGT/HLA Database. Thus, the embodiments described herein provide methodologies and systems for training a machine-learning model that both reduces the training complexity and improves the training performance. For example, the variant-coding sequences used for training can be selected and/or trimmed such that training is performed using variant-coding sequences having an amino acid length at or below a threshold amino acid length (e.g., nine (9) amino acids). Generating a training dataset that includes variant-coding sequences having a length equal to, or shorter than, the threshold amino acid length may reduce the overall training complexity as well as improve training and/or prediction performance (e.g., reduce variation in performance metrics per epoch to thereby improve prediction performance).

Accordingly, the techniques disclosed herein include machine-learning-based approaches for determining predicted amino acid-IPC interactions related to immunological activity associated with a peptide, such as a mutant peptide. A machine-learning model may generate an output comprising one or more predicted amino acid-IPC interactions. For example, the output may comprise one or more of: one or more interaction predictions, one or more interaction affinity predictions, or one or more immunogenicity predictions (i.e., a prediction relating to the ability of a peptide to provoke an immune response). An interaction prediction may include a prediction related to whether a peptide (e.g., a mutant peptide, including a given ordered set of amino acids as identified by a given variant-coding sequence) experiences one or more target interactions. In some embodiments, a target interaction can be the binding of a peptide to an IPC (e.g., an MHC molecule, a TCR), a peptide being presented by an MHC molecule at a cell surface, or another type of target interaction. An interaction affinity prediction may include a prediction of the affinity for one or more target interactions. For example, an interaction affinity prediction may indicate a binding affinity with respect to a peptide-MHC binding. An interaction (e.g., binding) affinity can be determined based on the tendency, strength, and/or stability of the interaction (e.g., binding). An immunogenicity prediction refers to predicting the ability of the peptide to elicit an immune response. The immune system recognizes the peptide as a non-self or foreign. Once recognized, the peptide stimulates the immune system to produce a response. This response can include the production of antibodies by B cells (humoral immunity) and the activation of T cells (cell-mediated immunity) to eliminate the cells presenting the peptide.

In some embodiments, the output may include or indicate an immunogenicity of a peptide or therapeutic antibody. For example, the output may predict whether a peptide will trigger an immune response in a particular subject or group of subjects. These immunogenicity predictions can be determined for each of a plurality of mutant peptides. In some embodiments, the immunogenicity predictions can be used to select or rank one or more mutant peptides and/or pharmaceutical compositions to be included in a vaccine and/or used in treatment. For example, without limitation, mutant peptides associated with high predicted binding affinity, a high probability of being presented at tumor cell surfaces, and/or high predicted immunogenicity can be selected for inclusion in a vaccine or use in a treatment.

The embodiments described herein provide methods and systems for using a machine-learning model to determine a predicted amino acid-IPC interaction indicative of the immunological activity related to amino acids (e.g., peptides) and immunoprotein complexes (IPCs). An IPC may comprise an MHC or a TCR. A set of amino acid sequences identified from at least one protein can be accessed. In some embodiments, the at least one protein is a therapeutic protein. In some embodiments, the at least one protein is present in a disease sample from a subject. An IPC sequence can be identified for an IPC of the subject and then accessed. A set of amino acid sequence representations are processed using one or more first processing blocks in a processing subsystem of a machine-learning model. The processing of the set of amino acid sequence representations may generate a set of transformed amino acid sequence representations. An IPC sequence representation can be processed using a second processing block in the processing subsystem to generate a transformed IPC sequence representation. In some embodiments, the BOS token embedding of each of the set of transformed amino acid sequence representations can be combined with the BOS token embedding of the transformed IPC sequence representation to generate composite representations. The method may generate an output comprising a predicted amino acid-IPC interaction that is determined based on the composition representations. The result (e.g., output) may comprise one or more of: one or more interaction predictions, one or more interaction affinity predictions, or one or more immunogenicity predictions for a corresponding amino acid-IPC combination. In some embodiments, a report is generated based on the output.

The techniques described herein provide numerous technical advantages. For example, the techniques described herein can avoid or reduce overfitting. Overfitting is an undesirable machine learning behavior that occurs when the machine learning model gives accurate outputs for training data but not for new data. Providing insufficient training data on the binding of peptides to MHC alleles for the implementation of a machine-learning model such as a transformer, can result in overfitting. A typical transformer can have a large number of parameters learned by the model from training data. In particular, data on the binding of peptides to MHC alleles can be challenging to obtain for several reasons, including a) high variability of MHC molecules, b) complexity of peptide binding, c) subject variation in MHC expression, d) ethical constraints and e) other technical limitations requiring specialized equipment and expertise. The collection of training data for other IPCs machine learning model predictions, e.g., TCRs, may suffer similar challenges.

Some of the embodiments illustrate computational techniques to train machine-learning models in a way that eliminates overfitting caused by insufficient training data. Such techniques include the use of a Protein Language Model (PLM) to process an IPC allele, e.g., an MHC allele, and infer useful and generalizable amino acid or residue features that can be used in the training process of a machine learning model. Thus reducing the likelihood of overfitting and improving the performance of the machine-learning model. For example, when an IPC allele sequence is processed by a PLM (e.g., to produce training data), the trained machine learning model can learn which residues or amino acids in a peptide-MHC binding complex are relevant or impact such a binding.

In some examples, the techniques described herein can advantageously incorporate information of the source protein from which the peptide is derived. Without incorporating information on the source protein, a model may lack useful protein/peptide features to generate the predictions described herein. By incorporating information on the source protein (e.g., via a protein sequence embedding generated by a PLM), the system can select peptides that are more likely to be presented by MHC molecules because the system can capture features such as: processing signals (i.e., flank signals produced when enzymes break down proteins into peptides), expression of the gene associated with the source protein, cellular localization of the source protein, and other suitable features that affect the presentation of a peptide by an MHC molecule and other interactions predicted by the implementations described herein. In some instances, source protein expression can be important because a source protein that is not sufficiently expressed in a subject will not result in sufficient peptides, even if such peptides can elicit an immunogenic effect. Cellular localization of the source protein is another feature that can be obtained by processing the source protein with the PLM. MHC I presentation can occur more often when the source protein located or generated from within the cell. On the other hand, proteins that are primarily extracellular (e.g., a protein that resides only in the vesicles) may be unlikely to be presented by MHC class I because they are in a different part of the cell. Conversely, proteins that are primarily extracellular (e.g., a protein that resides only in the vesicles) can be more likely to be presented by MHC II. Thus, for MHC I, it is preferred that the source protein is originated within the cell, and for MHC II, vesicle or extracellular proteins are preferred. In sum, there are many features related to the source protein that affect peptide presentation, which are advantageously incorporated in the techniques described herein.

In some embodiments, the techniques described herein can advantageously leverage a cross-attention module to incorporate MHC allele-specific binding information. MHC allele information can be valuable when processing the peptide data. For example, a peptide may contain multiple binding cores (i.e., specific peptide regions where binding occurs), which may bind to different MHC alleles. The system can use the cross-attention module to incorporate an MHC allele-specific vector into the processing of the peptide data. In this way, the system can predict the binding core information from peptides that have multiple binding cores that can bind to different MHC alleles and produce multiple binding core predictions corresponding to the multiple different alleles. In other words, techniques presented herein are not limited to predictions of one binding core per peptide. In some implementations, the machine learning models can predict more than one binding core per peptide.

In some examples, the techniques described herein can advantageously prevent over-prediction of a peptide amino acid position indicating the beginning of a binding core. For instance, a position zero (i.e., the start position) is unlikely to be the binding core start position of the peptide because the binding core is expected to be a portion (e.g., a 9-mer) of the peptide (e.g., a 20-mer) and to be surrounded by binding core flanks within the peptide. To mitigate the over-prediction problem, the techniques described herein can include a calibration step to remove the bias toward the position zero or other peptide position suffering from over-prediction. Before performing the calibration step, the system can first calculate model biases toward any single position in the peptide. The system can perform the calibration step by modifying an attention map by subtracting the model bias to remove model biases toward any single position in the peptide.

In some embodiments, the techniques described herein can advantageously use a dimensionality reduction module to process MHC data (e.g., the MHC sequence embedding) to prevent overfitting. For example, if a different type of model such as a neural network is trained to reduce the dimensionality of an input embedding, overfitting may occur. If the neural network is trained using the same set of MHC alleles used to train the PLM model, which typically is a relatively small set of MHC alleles, the neural network model may produce a correct output when processing an input embedding corresponding to an MHC allele in the training data but produce an incorrect output when processing an input embedding corresponding to a new MHC allele not included in the training data. The PCA technique, on the other hand, can be trained using a larger set of MHC alleles (e.g., the space of all alleles) and thus can efficiently and accurately reduce the dimension of an input embedding for a new allele not in the training dataset, thus avoiding the overfitting problem.

In the description that follows, it should be noted that the embodiments illustrated herein are not confined to the specific advantages disclosed above. This disclosure encompasses a variety of technical benefits and enhancements which are detailed throughout this written description. The embodiments are presented in a non-limiting manner, with the understanding that numerous modifications, variations, and refinements can be made without departing from the spirit and scope of the invention. The following description provides further technical advantages and novel aspects inherent in the presented embodiments, thereby offering a broader perspective on the applicability and utility of the disclosed invention.

The description below provides example implementations of these methods and systems in which an output (e.g., predicted amino acid-IPC interaction) can be used to plan for, design, and/or manufacture a treatment.

### Example Prediction System

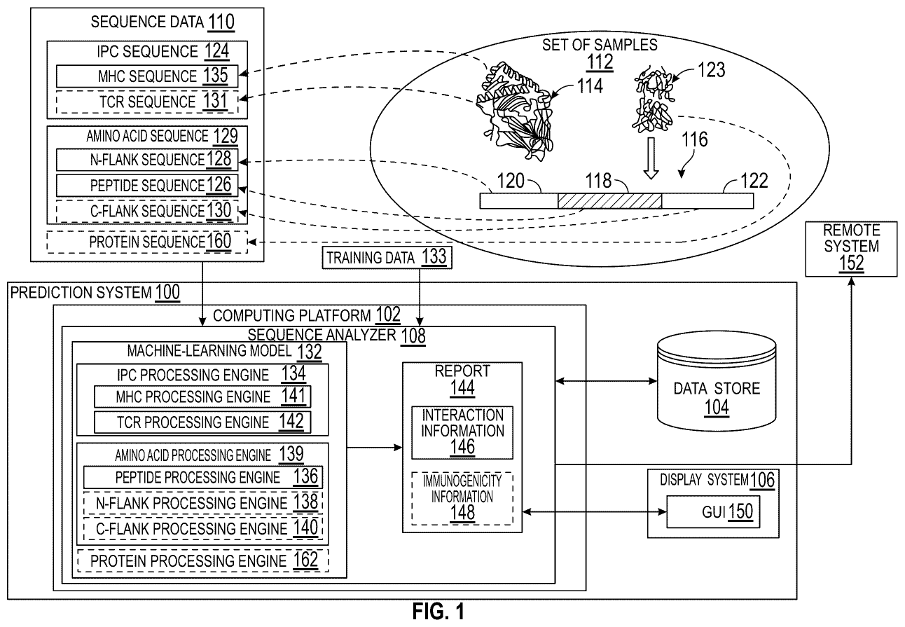

FIG. 1 is a block diagram of an example prediction system, in accordance with some embodiments. Prediction system 100 is used to determine a predicted amino acid-IPC interaction related to the immunological activity of peptides and, in particular, mutant peptides. The prediction system 100 includes computing platform 102, data store 104, and display system 106. Computing platform 102 may take various forms. In some embodiments, the computing platform 102 includes a single computer (or computer system) or multiple computers in communication with each other. In some embodiments, the computing platform 102 can be a cloud computing platform.

Data store 104 and display system 106 are each in communication with computing platform 102. In some examples, one or more of: data store 104 or display system 106 can be considered part of, or otherwise integrated with, computing platform 102. Thus, in some examples, computing platform 102, data store 104, and display system 106 can be separate components in communication with each other, but in other examples, some combination of these components can be integrated together. Communication between the different components can be implemented using any number of wired communications links, wireless communications links, optical communications links, or a combination thereof.

The prediction system 100 includes a sequence analyzer 108, which can be implemented using hardware, software, firmware, or a combination thereof. In some embodiments, the sequence analyzer 108 is implemented in the computing platform 102. The sequence analyzer 108 receives sequence data 110 for processing. For example, the sequence data 110 can be sent as input into the sequence analyzer 108, retrieved from the data store 104 or some other type of storage (e.g., cloud storage), accessed from cloud storage, or obtained in some other manner. In some cases, the sequence data 110 can be retrieved from the data store 104 in response to receiving user input entered by a user via an input device.

The sequence data 110 can be generated from processing of a set of samples 112. The set of samples 112 may take the form of one or more biological samples from one or more subjects (e.g., a diseased sample, a healthy sample, or a combination thereof). The set of samples 112 may include a sample obtained from a tumor of a subject. The tumor can be a manifestation of, for example, lung cancer, melanoma, breast cancer, ovarian cancer, prostate cancer kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T cell lymphocytic leukemia, non-small cell lung cancer, small-cell lung cancer, or a combination thereof.

A sample in the set of samples 112 may include, for example, various IPC molecules, various peptides, or a combination thereof. When the set of samples 112 includes a diseased sample, the peptides may include one or more mutant peptides (e.g., neoantigens). The IPC molecules may include, for example, various MHC molecules, various TCR molecules, or a combination thereof.

In some embodiments, the set of samples 112 includes immunoprotein complex (IPC) 114 (e.g., MHC Class I molecule, MHC Class II molecule, various TCR molecules, etc.). Further, the set of samples can include at least one protein 123 (i.e., the source protein). The amino acid chain 116 can be identified from the at least one protein 123 and can be a chain of amino acids that includes a peptide 118, an N-flank 120, and a C-flank 122. The amino acid chain 116 can be include or exclude the N-terminus between the peptide 118 and the N-flank 120, or the C-terminus between the peptide 118 and the C-flank 122. The peptide 118 is considered a mutant peptide when it includes one or more variants (e.g., one or more sequence variations) when compared to a corresponding reference sequence. The protein 123 is a source protein for the amino acid chain 116, which can be generated through proteolysis, which is the process by which proteins (e.g., the protein 123) are broken down into smaller polypeptides or amino acids. The protein 123 can be broken down into smaller polypeptides or amino acids by enzymatic cleavage, where specific enzymes called proteases cut the peptide bonds between amino acids in the protein 123.

The set of samples 112 can be processed to generate the sequence data 110. In some embodiments, multiple samples in the set of samples 112 can be processed at different times. In some embodiments, the prediction system 100 includes a sample analyzer that is used in processing the set of samples 112 to generate the sequence data 110. The sequence data 110 includes, for example, at least one amino acid sequence 129 and at least one immunoprotein complex (IPC) sequence 124 (e.g., one IPC sequence 124 corresponding to IPC 114). The amino acid sequence 129 may comprise one or more of: a peptide sequence 126 (e.g., one peptide sequence 126 corresponding to peptide 118), an amino-terminal flanking (N-flank) sequence 128 (e.g., one N-flank sequence 128 corresponding to N-flank 120), or a carboxy-terminal flanking (C-flank) sequence 130 (e.g., one C-flank sequence 130 corresponding to C-flank 122). One or more sub-sequences of amino acid sequence 129 (e.g., peptide sequence 126, N-flank sequence 128, and C-flank sequence 130) can be processed separately or as a single sequence.

When immunoprotein complex 114 is an MHC, IPC sequence 124 can be, for example, an MHC sequence 135 that characterizes at least a portion of the MHC. When immunoprotein complex 114 is a TCR, IPC sequence 124 can be, for example, a TCR sequence 131 that characterizes at least a portion of the TCR. In some embodiments, IPC sequence 124 may include both an MHC sequence 135 characterizing at least a portion of an MHC molecule and a TCR sequence 131 characterizing at least a portion of a TCR molecule. In some embodiments, the sequence data 110 may include IPC sequence 124 in the form of an MHC sequence 135 characterizing at least a portion of an MHC molecule, as well as a separate TCR sequence 131 characterizing at least a portion of a TCR.

Protein sequence 160 characterizes at least a portion of the protein 123. In some embodiments, the protein sequence 160 can be identified by performing a reverse lookup in a database (e.g., the UniProt database) based on the mutant peptide data (e.g., IPC sequence 124) obtained from the sample.

Peptide sequence 126 characterizes at least a portion of the peptide 118. N-flank sequence 128 characterizes at least a portion of the N-flank 120. In some embodiments, when the number of amino acids (or amino acid residues) upstream from the N-terminus is large, the corresponding sequence for N-flank 120 can be trimmed to generate the N-flank sequence 128. C-flank sequence 130 characterizes at least a portion of the C-flank 122. In some embodiments, when the number of amino acids (or amino acid residues) downstream from the C-terminus is large, the corresponding sequence for C-flank 122 can be trimmed to generate the C-flank sequence 130.

Sequence analyzer 108 receives the sequence data 110 as input for processing. The sequence analyzer 108 includes the machine-learning model 132 that processes the sequence data 110. In some embodiments, the sequence data 110 is sent directly into the machine-learning model 132 for processing. In some embodiments, the sequence analyzer 108 preprocesses the sequence data 110 prior to sending the sequence data 110 into machine-learning model 132 for processing. Pre-processing the sequence data 110 may include appending a beginning-of-sequence (BOS) token to each of a plurality of sequences in the sequence data 110. A BOS token appended to a peptide sequence may serve as an additional data structure that can be used to represent the properties of the peptide which, can be used to determine presentation likelihood, binding affinity, or prediction of immunogenicity of the corresponding peptide sequence 126. This BOS token may indicate interaction information such as whether the peptide will be presented by an allele/allotype, binding affinity, immunogenicity, or any other suitable purpose for which machine-learning model 132 has been trained.

The machine-learning model 132 can be implemented in any of a number of different ways. In some embodiments, the machine-learning model 132 can be any type of model that uses a set of element-focused scores that represent binding cores of the set of amino acid sequence representations. Machine-learning model 132 can be used in either a training mode or a prediction mode. In the training mode, the machine-learning model 132 is trained using training data 133. Examples of the training data 133 are described in more detail below. The machine-learning model 132 is trained such that it can be used in the prediction mode.

The machine-learning model 132 processes the IPC sequence 124 via an IPC processing engine 134 and the amino acid sequence 129 via an amino acid processing engine 139. The separate processing engines for IPC and amino acid enable improved predictive performance of the machine-learning model 132. In some embodiments, the machine-learning model 132 processes one or more of: a MHC sequence 135 via an MHC processing engine 141 or a TCR sequence 131 via a TCR processing engine 142. In some embodiments, the machine-learning model 132 processes one or more of: a peptide sequence 126 via a peptide processing engine 136, an N-flank sequence 128 via an N-flank processing engine 138, or a C-flank sequence 130 via a C-flank processing engine 140. In some embodiments, the machine-learning model 132 processes the protein sequence 160 via a protein processing engine 162. Examples of implementations for these different processing engines are described in greater detail below.

As used herein, the terms “processing engine” and “engine” identify at least one software component and/or a combination of at least one software component and at least one hardware component which are designed/programmed/configured to interact and/or communicate data to other software and/or hardware components including but not limited to other processing engines.

The machine-learning model 132 processes the sequence data 110 to generate an output that is used to generate a report 144. The report 144 may include the exact output of the machine-learning model 132, a transformed or filtered version of the output, or both. In some cases, the report 144 may include notifications, recommendations, alerts, or other information generated by the sequence analyzer 108 based on the output of the machine-learning model 132.

The report 144 can be an output that includes, for example, information about immunological activity of interest with respect to one or more peptides (e.g., one or more mutant peptides). For example, the report 144 may include information about the immunological activity relating to the amino acid 116 (e.g., peptide 118, N-flank 120, C-flank 122, etc.) and IPC 114 (e.g., MHC-I, MHC-II, TCR, etc.). The report 144 may include, for example, interaction information 146 (e.g., an interaction affinity prediction that predicts a binding affinity between a peptide and an MHC, or an interaction prediction that predicts whether an MHC allele or allotype will present a peptide at a cell surface), immunogenicity information 148 (e.g., an immunogenicity prediction that predicts the ability of a peptide to provoke an immune response with respect to an MHC), or both. The interaction information 146 may provide predictions about a selected set of interactions between the amino acid 116 and the IPC 114. The immunogenicity information 148 may provide predictions about the immunogenicity of amino acid 116 (e.g., including the immunogenicity of the peptide 118).

In some embodiments, a report 144 can be displayed on a graphical user interface (GUI) 150 on the display system 106. A user may view and/or interact with the report 144 via the graphical user interface 150. In some embodiments, the user may use the report 144 to make decisions about the treatment of a subject from which at least one of the set of samples 112 was obtained (or collected).

In some embodiments, the prediction system 100 sends the report 144 to the remote system 152 (e.g., wirelessly). The remote system 152 can be a cloud computing platform, cloud storage, another computer system, a user device (e.g., a smartphone, a tablet, a laptop, etc.), or some other type of platform. In some embodiments, the remote system 152 can be a treatment manufacturing system (or machine) or a portion thereof.

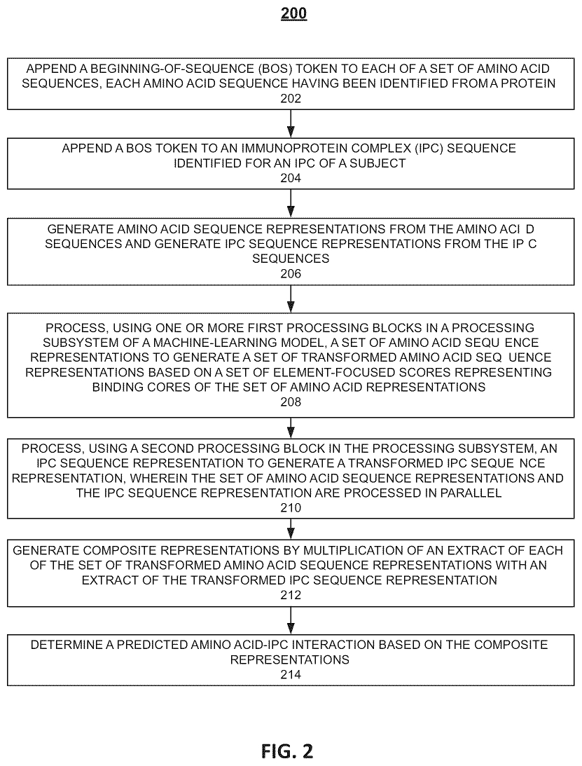

FIG. 2 is a flowchart of an example process (e.g., computer-implemented method) for predicting an amino acid-IPC interaction using a machine-learning model, in accordance with some embodiments. Process 200 can be implemented using the prediction system 100 described in FIG. 1. For example, process 200 can be implemented using the sequence analyzer 108 and the machine-learning model 132 in FIG. 1.

Process 200 may include, for example, step 202. Step 202 includes appending a BOS token to each amino acid sequence in a set of amino acid sequences. Each of the amino acid sequences of the set may have been identified from at least one protein. In some embodiments, the at least one protein is a therapeutic protein. In some embodiments, the at least one protein is present in a disease sample from a subject. As one non-limiting example, the disease sample can be a tumor cell biopsy. Additionally or alternatively, the disease sample may include cancer, tissue, or both. In some embodiments, each of the amino acid sequences comprises one or more of: an amino-terminal flanking (N-flank) sequence or a carboxy-terminal flanking (C-flank) sequence.

Step 204 includes appending a BOS token to an IPC sequence identified for an IPC of a subject. In some embodiments, the IPC of the subject is an MHC. The MHC may comprise MHC class II (MHC-II) or MHC class I (MHC-I). In some embodiments, the IPC of the subject is a TCR.

Step 206 includes generating a set of amino acid sequence representations (e.g., embeddings) for each of the amino acid sequences and generating a set of IPC sequence representations (e.g., embeddings) from the set of IPC sequences. An embedding module may generate each of the sequence representations by creating an embedding for each element (e.g., a single amino acid or a single nucleic acid) of the sequence to represent features of the element as a low-dimensional feature vector. The embedding module may also generate, as part of the sequence representation, positional embeddings representing each absolute position (corresponding to one of the amino acids or to one of the nucleic acids). The embedding corresponding to the BOS token may have a length equal to the number of features corresponding to each individual sequence element represented in the respective sequence to which the BOS token was appended.

Step 208 includes processing the set of amino acid sequence representations using one or more processing blocks in a processing subsystem of a machine-learning model. Processing the set of amino acid sequence representations through each of one or more transformer stages may generate a set of transformed amino acid sequence representations stored in the embedding corresponding to the BOS token. Sequences having simple molecular structures (e.g., N-flank/C-flank sequence) may only require a single transformer stage, whereas sequences having a more complex molecular structure (e.g., peptide sequences) may require multiple transformer stages. The transformer stages compute information about a frequency of correlations between each pair of amino acids in the input amino acid sequence representation and store information about the pairwise correlations in the transformer output. The transformer stages may thereby store information about pairwise correlations between amino acids as well as the absolute position of each amino acid within each amino acid sequence (e.g., from the positional embedding) into the embedding corresponding to the BOS token within the transformed amino acid sequence representation. The set of transformed amino acid sequence representations may comprise a set of MHC-binding representations. The set of transformed amino acid sequence representations may comprise one or more of: a set of amino-terminal flanking (N-flank) sequence representations, a set of carboxy-terminal flanking (C-flank) sequence representations, or a set of combined N-flank/C-flank sequence representations. The N-flank/C-flank sequence representation(s) can be processed in parallel with the amino acid sequence representations by using independent processing blocks. Each of the processing blocks may utilize an attention mechanism in order to determine an element-focused score.

Step 210 includes processing the IPC sequence representation using a second processing block in the processing subsystem, which may operate independently of and in parallel with the first processing block, to generate a transformed IPC sequence representation stored in the embedding corresponding to the BOS token. Information about each IPC sequence may thereby be stored in the embedding corresponding to the BOS token of the transformed IPC sequence representation. In some embodiments, the IPC sequence may comprise a TCR sequence in addition to an MHC sequence—in such embodiments, a TCR sequence representation can be generated separately from the MHC sequence representation, and the two sequence representations can be processed in parallel using independent processing blocks. Each of the processing blocks may utilize an attention mechanism in order to determine an element-focused score.

Step 212 includes generating a composite representation by combining each of the BOS token embeddings for each of the transformed amino acid sequence representations with the BOS token embedding for the transformed IPC sequence representation. The combining process can be an element-wise multiplication, an element-wise addition, or computation of a dot product. In some embodiments, when the amino acid sequence includes both a peptide sequence and an N-flank sequence and/or a C-flank sequence, prior to the combination step, the BOS token embedding corresponding to the peptide sequence can be concatenated with the BOS token embedding corresponding to the N-flank/C-flank sequence representation(s).

Step 214 includes determining a predicted amino acid-IPC interaction based on the composite representations. The predicted amino acid-IPC interaction may include one or more of: one or more interaction predictions, one or more interaction affinity predictions, or one or more immunogenicity predictions for one or more corresponding amino acid-IPC combinations.

In some embodiments, an output (e.g., a report) can be generated. The output can be based on the predicted amino acid-IPC interaction. The output can be used to facilitate the design and/or manufacture of a vaccine, treatment, and/or treatment plan. For example, a report may identify a subset of the set of peptides (included in a set of amino acid sequences), or provide an indication of which peptides to select for the subset of peptides for use in creating a treatment for the subject. The treatment can be, e.g., the subset of peptides, a precursor for each of the subset of peptides, or some other form.

### Example Architectures of a Machine-Learning Model

The machine-learning model 132 of the embodiments described herein may include multiple subsystems (or subnetworks). Each of the multiple subsystems can include an encoder, a transformer encoder, and/or one or more processing layers. In some instances, the machine-learning model 132 can be configured to learn alignments (e.g., between an amino acid sequence and an IPC sequence, between a peptide sequence and an MHC sequence, between an MHC sequence and a TCR sequence, MHC-Peptide complex and a TCR, Peptide Sequence and TCR sequence). The alignments can be learned and performed using an alignment score function such as, for example, a content-based function, an additive function, a location-based function, a dot-product function, and/or a scaled dot-product function.

Machine-learning model 132 may include one or more encoders configured to, for example, transform an element of the input sequences (e.g., an amino acid sequence, a nucleic acid sequence, a codon sequence, etc.) based on the other elements of the input sequences. An encoder can be a transformer encoder.

The machine-learning model 132 may include one or more processing layers such as self-attention layers or convolution layers, or a neural network such as a long-short term memory unit (LSTM), recurrent structure, or recurrent component. The machine-learning model 132 can implement, for example, one or more self-attention layers. Machine-learning model 132 can use a self-attention mechanism, global attention mechanism, soft attention mechanism, local attention mechanism, and/or hard attention mechanism. In some instances, the machine-learning model 132 does not include any convolutional layer, any recurrent structure, any LSTM unit, and/or any recurrent component. In some instances, the machine-learning model 132 is not a recurrent machine-learning model and/or does not include a recurrent neural network. In some instances, the machine-learning model 132 includes a recurrent neural network and/or may use positional encoding to handle sliding windows of sequence elements across one or more sequences. In some instances, the machine-learning model 132 is not a convolutional machine-learning model and/or does not include a convolutional neural network.

The machine-learning model 132 may include processing blocks, such one or more first processing blocks used to process one or more amino acid sequence representations independent from a second processing block used to process an IPC sequence representation. In some embodiments, the second processing block may process part or all of an IPC sequence representation (e.g., an MHC pseudosequence). The independence of these processing blocks can facilitate parallel processing when using the machine-learning model 132. Further, the independence may improve the performance (e.g., accuracy of predictions) of the machine-learning model 132.

The machine-learning model 132 can be configured such that an output value at any given layer depends not only on a corresponding input value but also on one or more (e.g., all) other input values. Thus, the machine-learning model 132, a loss function, and/or an optimization function can be configured to optimize an output corresponding to a single position representing a degree to which a given IPC (e.g., MHC molecule) (represented by a corresponding input) will bind to a given peptide (represented by another corresponding input) and/or trigger immunogenicity in response to the given peptide. In some instances, the loss function can comprise supervised loss function such as binary cross entropy or unsupervised loss functions. In some instances, the unsupervised loss functions can include a contrastive loss or regularization losses (e.g., L1/L2 losses applied on a peptide representation to make residue changes more continuous). In some instances, the loss function can comprise auxiliary loss functions. In such instances, the auxiliary loss function can be used alongside a main loss function to train the machine learning model 132. Accordingly, in some instances, the auxiliary loss function can improve the learning process by adding additional information or constraints. In some instances, any of a plurality of outputs of transformer encoders may represent such an occurrence probability. The machine-learning model 132 can be trained accordingly. In some instances, an endpoint (e.g., surplus endpoint) may represent (in response to training) a binding affinity, presentation (eluted ligand or EL), and/or immunogenicity probability or likelihood. Aggregated outputs can be, for example, fed to another layer, subsystem, or processing block (e.g., that includes one or more of: a processing layer such as a self-attention layer, or an encoder such as a transformer encoder).

In some instances, one, two, or all dimensions of an output from another layer and/or another subsystem or processing block is the same size as the input fed to the other layer and/or other subsystem or processing block. In some instances, an input fed to this other layer and/or other subsystem or processing block has a length along one axis that is greater than or equal to a sum of one or more of: a number of amino acids in an IPC sequence, a number of amino acids in a peptide sequence, a number of amino acids in an N-flank sequence, or a number of amino acid in a C-flank. In some instances, the length of the input is one longer than the total number of amino acids. The length of the input along the one axis may exceed the summed count of amino acids when, for example, an additional feature vector (e.g., feature vector corresponding to a BOS token, or a token representing the IPC type) is appended to the amino acid-specific feature values. Another dimension of the input can include a number of features (e.g., determined via a hyperparameter). An output generated by the other layer and/or other subsystem or processing block may have the same size as the input.

A subset of values of the output generated by the other layer and/or other subnetwork can be processed by another neural network (e.g., a fully connected feedforward network). The subset of values may include a 1-dimensional vector of values that may correspond to one set of feature values. For example, the 1-dimensional vector may correspond to feature values associated with a BOS token.

In some embodiments, a neural network within the machine-learning model 132 can be configured to output one or more results. The one or more results can include, for example, a numeric result, binary result, and/or categorical result. Each of the one or more results can predict whether and/or an extent to which an amino acid (e.g., a peptide) and an IPC undergo a reaction of a particular type (e.g., bind together). The machine-learning model 132 may include one or more activation layers to produce an intermediate result (e.g., to transform a real-number interim value into a binary and/or categorical output). The machine-learning model 132 can be trained to generate multiple types of predictions (e.g., interaction predictions, interaction affinity predictions, and/or immunogenicity predictions). In some instances, a prediction can be binary or categorical. Other predictions can be non-binary or non-categorical. For example, a prediction can be scalar.

Machine-learning model 132 may include and/or can be included within an ensemble model. The ensemble model may include multiple (e.g., identical) sub-models that can be trained using different portions of the training data set.

### Example Configuration for a Machine-Learning Model

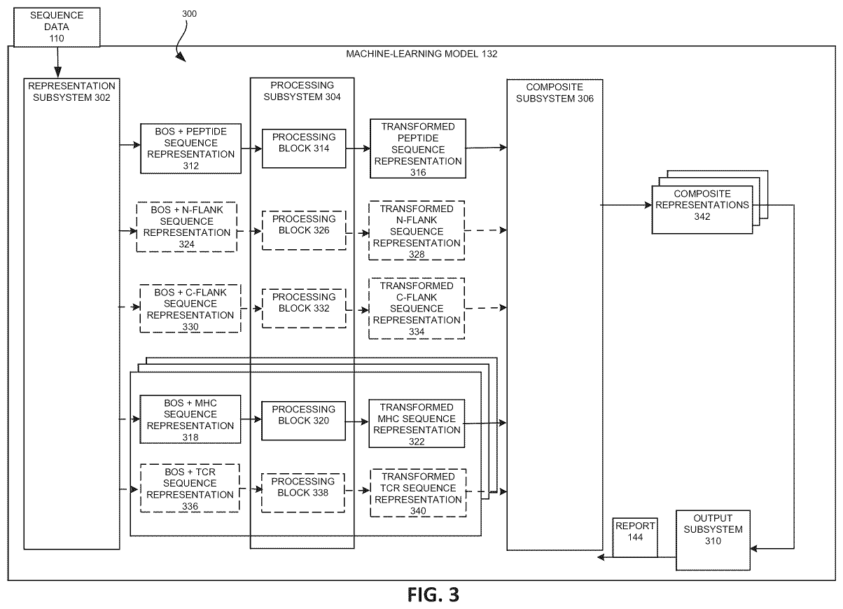

FIG. 3 is a schematic diagram of an example configuration for the machine-learning model 132 from FIG. 1, in accordance with some embodiments. The machine-learning model 132 is described with continuing reference to FIG. 1. The machine-learning model 132 may have configuration 300 comprising representation subsystem 302, processing subsystem 304, composite subsystem 306, and output subsystem 310. One or more subsystems within the machine-learning model 132 may comprise one or more blocks, one or more sub-blocks, one or more layers, or a combination thereof. One or more blocks within the machine-learning model 132 may comprise one or more sub-blocks, one or more layers, or a combination thereof. One or more sub-blocks of the machine-learning model 132 may comprise one or more layers (or units).

In some embodiments, representation subsystem 302 receives the sequence data 110 as input and passes it through a tokenizer (not shown) that converts each letter of the sequence into a token (e.g., a unique integer stored in a lookup table in association with the letter). The tokenizer may append a BOS token to one or more of the sequences in sequence data 110. Representation subsystem 302 may generate a sequence representation for each of the sequence in the sequence data 110. A sequence representation may include, for example, a stack of feature vectors corresponding to a sequence of sequence elements, each sequence element representing or identifying one or more amino acids, one or more nucleic acids in the sequence corresponding to the sequence representation, or the BOS token. For example, each amino acid in the sequence can be represented by a unique feature vector, and the BOS token appended to the amino acid sequence may likewise be represented by a unique feature vector. The IPC sequence representation may comprise six to twelve MHC alleles/allotypes of a given subject, and the processing subsystem 304 may generate up to twelve transformed MHC sequence representations, corresponding to one MHC transformed sequence representation for each MHC allotype in combination with a particular peptide sequence. Amino acid sequence 129 may comprise one or more of: a peptide sequence 126, an n-Flank sequence 128, or a c-Flank sequence 130, and the processing subsystem 304 may generate one amino acid sequence representation for each of the amino acid sequences. In order to normalize across different sequence lengths, the stack of feature vectors can be padded to a standard sequence length (SL), for example, 39 vectors.

Processing subsystem 304 may receive one or more sequence representations (e.g., a set of amino acid sequence representations, an IPC sequence representation) as input, processes these sequence representations in one or more transformer stages, and generate transformed sequence representations (e.g., a set of transformed amino acid sequence representations, a transformed IPC sequence representation) that are sent into composite subsystem 306. The processing subsystem 304 comprises one or more processing blocks. A processing block may include one or more processing layers (e.g., attention layers). Various transformer stages of processing subsystem 304 may thereby store information about each sequence into the embedding corresponding to the BOS token within the sequence representation.

In some embodiments, the representation subsystem 302 and/or the processing subsystem 304 can be configured to process subsequences of the amino acid sequences in parallel, using independent processing engines. For example, peptide processing engine 136 in FIG. 1 may include (1) the representation subsystem 302 processing a peptide sequence 126 appended with a BOS token to generate a BOS+ (plus) peptide sequence representation 312, followed by (2) processing block 314 in processing subsystem 304 processing the BOS+peptide sequence representation 312 to generate a transformed peptide sequence representation 316. In this example, N-flank processing engine 138 in FIG. 1 can be executed independently and in parallel by (1) the representation subsystem 302 processing an N-flank sequence 128 appended with a BOS token to generate a BOS+N-flank sequence representation 324, followed by (2) processing block 326 in the processing subsystem 304 processing the BOS+N-flank sequence representation 324 to generate a transformed N-flank sequence representation 328. In this example, C-flank processing engine 140 in FIG. 1 can be executed independently and in parallel by (1) the representation subsystem 302 processing a C-flank sequence 130 appended with a BOS token to generate a BOS+C-flank sequence representation 330, followed by (2) processing block 332 in the processing subsystem 304 processing the BOS+C-flank sequence representation 330 to generate a transformed C-flank sequence representation 334.

Similarly, the representation subsystem 302 and the processing subsystem 304 can be configured to process subsequences of the IPC sequence (e.g., MHC sequence, TCR sequence) in parallel, using independent processing engines. For example, MHC processing engine 141 in FIG. 1 may include (1) the representation subsystem 302 processing an MHC sequence 135 appended with a BOS token to generate a BOS+MHC sequence representation 318, followed by (2) processing block 320 in processing subsystem 304 processing the BOS+MHC sequence representation 318 to generate a transformed MHC sequence representation 322. In this example, TCR processing engine 142 in FIG. 1 can be executed independently and in parallel by (1) the representation subsystem 302 processing an TCR sequence 131 appended with a BOS token to generate a BOS+TCR sequence representation 336, followed by (2) processing block 338 in the processing subsystem 304 processing the BOS+TCR sequence representation 336 to generate a transformed TCR sequence representation 340.

In some embodiments, certain subsequence representations generated by representation subsystem 302 can be combined prior to appending a BOS token. For example, an N-flank sequence representation can be appended with a C-flank representation prior to appending a single BOS token to the combined sequence representation. In such embodiments, processing subsystem 304 processing the combined sequence representation may correspondingly generate a single transformed sequence representation.

The composite subsystem 306 generates composite representations 342 by combining the embedding corresponding to the BOS token of the transformed amino acid sequence representation with the embedding corresponding to the BOS token of each of the set of transformed IPC sequence representations. The composite subsystem 306 may combine the embedding corresponding to the BOS token of the transformed peptide sequence representation 316 with a set of embeddings corresponding to the BOS tokens of the transformed MHC sequence representations 322 to generate composite representations 342. In some embodiments, the combining process may comprise element-wise multiplication of the transformed peptide BOS token embedding by the set of transformed MHC BOS token embeddings. Element-wise multiplication is beneficial as it forces the latent spaces of the various components to cluster into complementary regions of the latent space. One consequence is that peptides that bind to a particular MHC result in embeddings in the same region (with similar effects with TCR and peptide sequences). In some embodiments, the combining process may comprise element-wise addition of the transformed peptide BOS token embedding with the set of transformed MHC BOS token embeddings, or computation of a dot product thereof.

Embodiments of the disclosure may include the set of transformed amino acid sequence representations comprising one or more of: a set of transformed N-flank sequence representations 328, a set of transformed peptide sequence representations 316, or a set of transformed C-flank sequence representations 334. In some embodiments, the set of transformed IPC sequence representations may comprise one or more of: a set of transformed MHC sequence representations 322 or a set of transformed TCR sequence representations 340. The composite subsystem 306 combines the BOS token embeddings of the transformed amino acid sequence representations by one or more of the BOS token embeddings (including all) of the transformed IPC sequence representations. The combining step may include multiplications or additions of the BOS token embeddings, such as element-wise multiplications of transformed amino acid BOS token embeddings (comprising one or more of: a transformed peptide BOS token embedding, a transformed N-flank BOS token embedding, or a transformed C-flank BOS token embedding) by transformed IPC BOS token embeddings (comprising one or more of: a transformed MHC BOS token embedding and a transformed TCR BOS token embedding). In some embodiments, the combining step may comprise dot product computations and/or element-wise additions instead of or in addition to element-wise multiplication.

The composite representations 342 can be processed by an output subsystem 310, and a predicted amino acid-IPC interaction can be determined based on the composite representations 342. In some embodiments, the predicted amino acid-IPC interaction can be based on one or more composite representations selected among the composite representations 342. In some embodiments, a report 144 comprising the selected one or more predicted amino acid-IPC interactions can be generated.

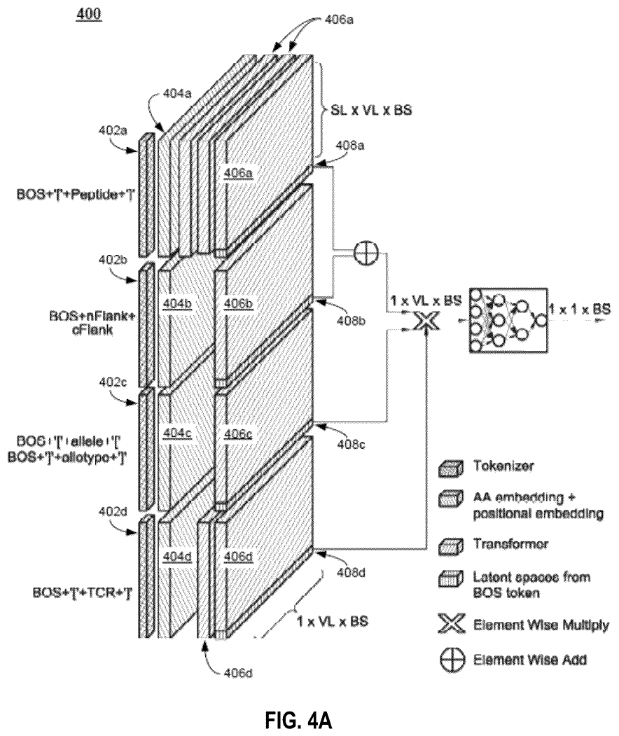

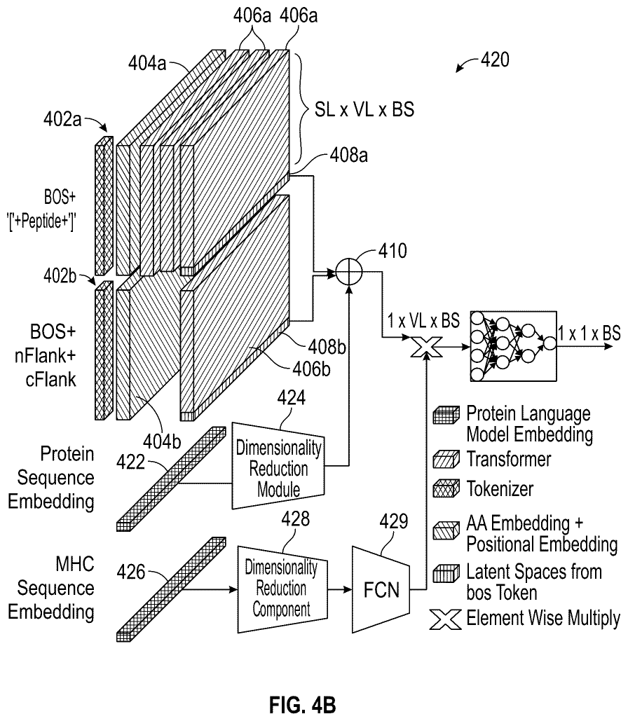

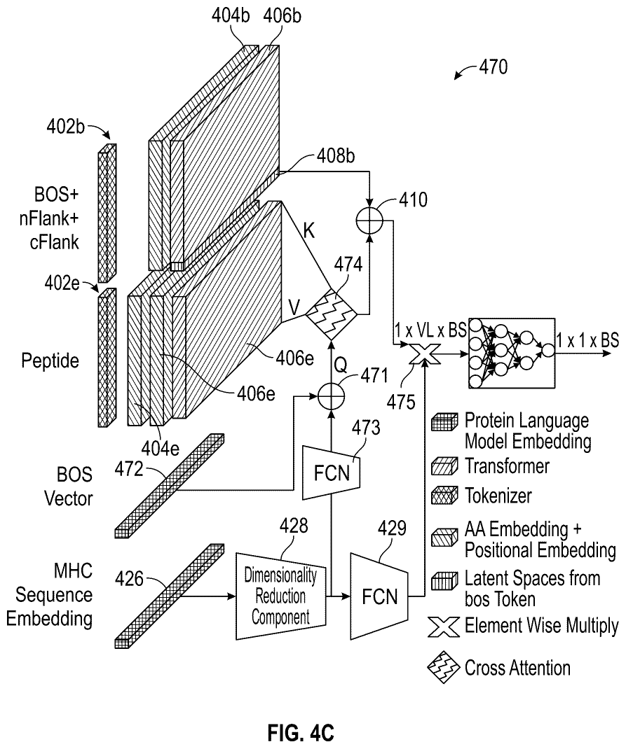

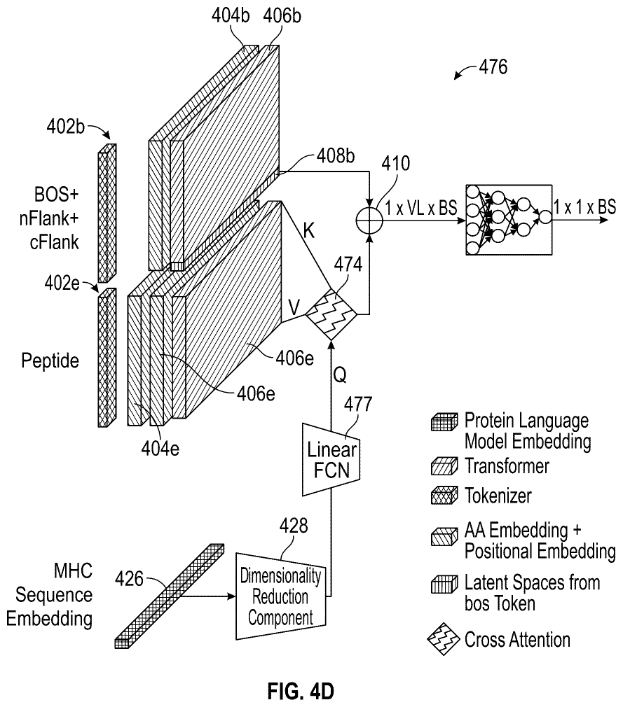

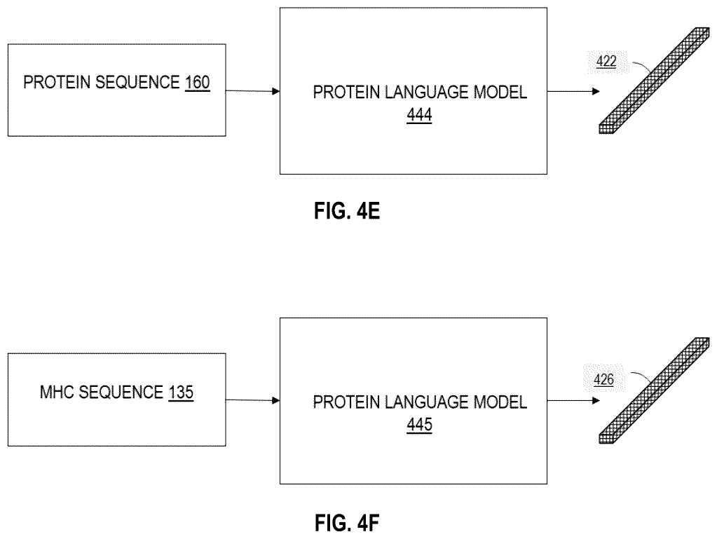

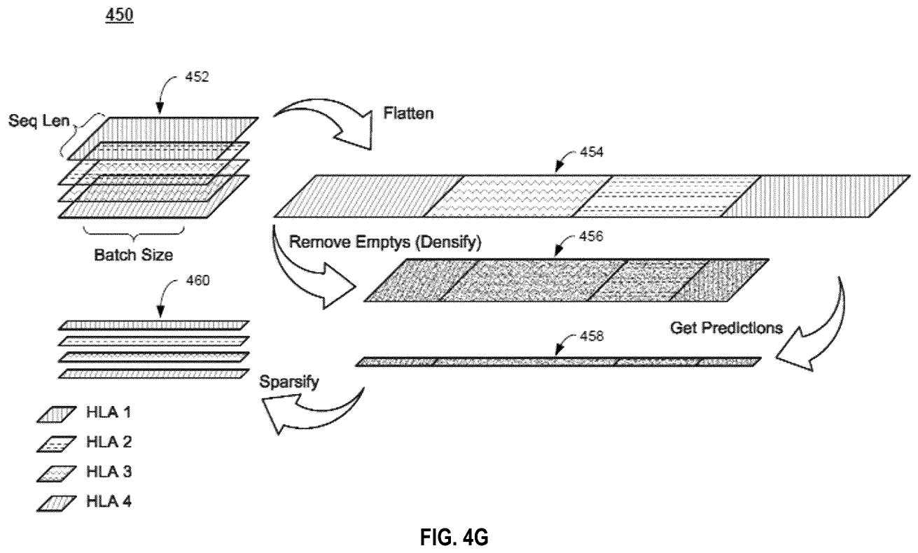

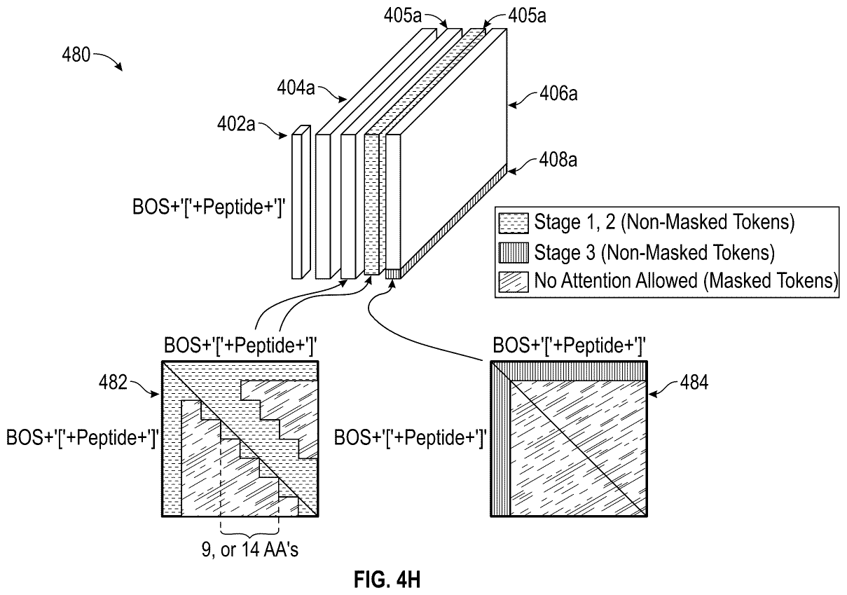

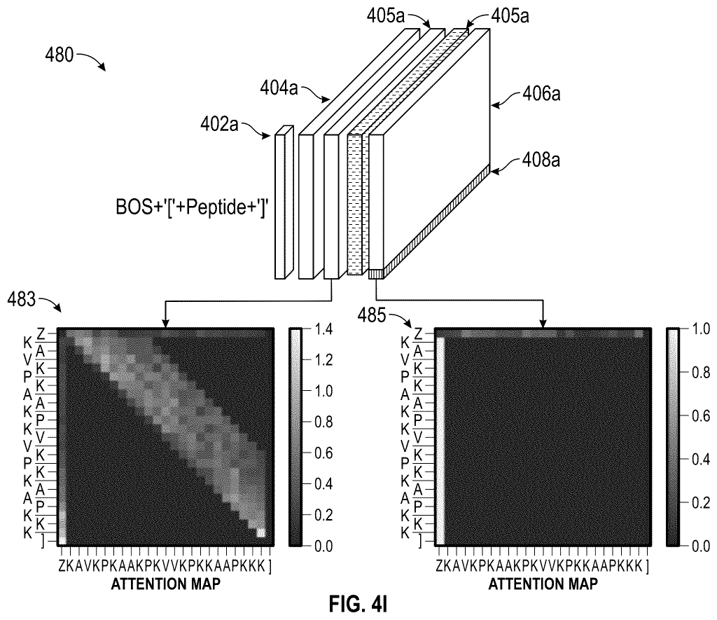

FIGS. 4A-D illustrate examples of workflows demonstrating various possible combinations of peptide data processing, nFlank/cFlank data processing, MHC data processing, TCR data processing, and/or protein data processing for obtaining one or more interaction predictions, one or more interaction affinity predictions, and/or one or more immunogenicity predictions, in accordance with some embodiments. The predictions can include, among other things, an interaction affinity prediction for a peptide-IPC combination that predicts a binding affinity between a peptide and MHC molecule; an interaction prediction for the peptide-IPC combination, for example, predicting whether an MHC molecule will present the peptide at a cell surface; or an immunogenicity prediction for the peptide-IPC combination that predicts an ability of the peptide to provoke an immune response. As described in detail below, the processing of MHC data can involve processing of a BOS token-appended MHC sequence using one or more transformer stages (e.g., FIG. 4A) or alternatively involve processing of an MHC sequence embedding generated using a protein language model (e.g., FIGS. 4B, 4C, 4D). The processing of peptide data can involve processing of a BOS token-appended peptide sequence using one or more transformer stages (e.g., FIGS. 4A and 4B) or alternatively involve processing of a peptide sequence not appended with a BOS token using a cross-attention module (e.g., FIGS. 4C and 4D). Optionally, any workflow may additionally incorporate the processing of protein data (e.g., FIG. 4B) or alternatively may not incorporate the processing protein data (e.g., FIG. 4A, 4C, 4D). Optionally, any workflow may additionally incorporate the processing of TCR data (e.g., FIG. 4A) or alternatively may not incorporate TCR data (e.g., FIG. 4B, 4C, 4D).

It should be appreciated that the workflows in FIGS. 4A, 4B, 4C, and 4D are merely examples, and that the present disclosure encompasses any workflow involving a combination of the processing of MHC data and the processing of peptide data for obtaining one or more interaction predictions, one or more interaction affinity predictions, and/or one or more immunogenicity predictions. In an example workflow, the processing of MHC data can involve processing of a BOS token-appended MHC sequence using one or more transformer stages (e.g., FIG. 4A) or alternatively involve processing of an MHC sequence embedding generated using a protein language model (e.g., FIGS. 4B, 4C, 4D). In the example workflow, the processing of peptide data can involve processing of a BOS token-appended peptide sequence using one or more transformer stages (e.g., FIGS. 4A and 4B) or alternatively involve processing of a peptide sequence not appended with a BOS token using a cross-attention module (e.g., FIGS. 4C and 4D). The example workflow may additionally incorporate the processing of protein data (e.g., FIG. 4B) or alternatively may not incorporate the processing protein data (e.g., FIG. 4A, 4C, 4D). The example workflow may additionally incorporate the processing of TCR data (e.g., FIG. 4A) or alternatively may not incorporate TCR data (e.g., FIG. 4B, 4C, 4D).

FIG. 4A illustrates an example workflow 400 for obtaining one or more interaction predictions, one or more interaction affinity predictions, and/or one or more immunogenicity predictions, in accordance with some embodiments. A tokenizer (not depicted) can create a tokenized sequence comprising a BOS token-appended peptide sequence 402a, a BOS token-appended nFlank+cFlank sequence 402b, a BOS token-appended MHC sequence 402c, and a BOS token-appended TCR sequence 402d. An embedding module can generate a sequence representation. Specifically, the embedding module can generate (e.g., via representation subsystem 302) a BOS+peptide sequence representation 404a (which may correspond to 312 in FIG. 3) based on the BOS token-appended peptide sequence 402a, a BOS+nFlank+cFlank sequence representation 404b which represents a combined version of BOS+N-flank sequence representation (which may correspond to 324 in FIG. 3) and BOS+C-flank sequence representation (which may correspond to 330 in FIG. 3) based on the BOS token-appended nFlank+cFlank sequence 402b, a BOS+MHC sequence representations 404c (which may correspond to 318 in FIG. 3) based on the BOS token-appended MHC sequence 402c, and a BOS+TCR sequence representation 404d (which may correspond to 336) based on BOS token-appended TCR sequence 402d. Transformed version(s) 406a-d (e.g., transformed BOS+peptide sequence representations 316 and 406a, transformed BOS+nFlank+cFlank sequence representation 406b which represents a combined version of BOS+N-flank sequence representation 328 and BOS+C-flank sequence representation 334, transformed BOS+MHC sequence representations 322 and 406c, or transformed BOS+TCR sequence representations 340 and 406d) of each of sequence representation 404a-d can be generated using one or more transformer stages in processing subsystem 304. During the transformer stages, BOS token embeddings 408a-d are also generated as part of transformed sequence representations 406a-d (e.g., BOS token embedding 408a of transformed BOS+peptide sequence representations 316 and 406a, BOS token embedding 408b of transformed BOS+nFlank+cFlank sequence representation 406b, BOS token embedding 408c of transformed BOS+MHC sequence representations 322 and 406c, or BOS token embedding 408d of transformed BOS+TCR sequence representations 340 and 406d). Transformed BOS token embeddings 408a-d extract information about the sequence (e.g., information about pairwise correlations and position) into the embedding corresponding to the BOS token appended to the sequence. Each of the transformed BOS token embeddings 408a-d can represent an entire sequence via a single vector rather than multiple vectors, thus making the sequence easier to interpret. A composite of the BOS token embeddings 408a-d may then be generated by composite subsystem 306 as described in FIG. 3, and then a final output generated by output subsystem 310 also described in FIG. 3. Each of the sequence representations (e.g., embeddings) 404a-d and the transformed sequence representations 406a-d can be of uniform dimensions, comprising a subsequence length (SL), vector length (VL), and batch size (BS). As shown in FIG. 4A, the dimensions of BOS token embeddings 408a-d have a subsequence length equal to 1, since they were generated from a single BOS token appended to each amino acid or IPC subsequence.

As shown in FIG. 4A, in peptide processing engine 136 (see FIG. 1), BOS token-appended peptide sequence 402a is created by the tokenizer (e.g., in representation subsystem 302) and then used by the embedding module (e.g., in representation subsystem 302) in order to generate peptide sequence representation 404a, from which several transformation stages result in transformed peptide sequence representation 406a, including transformed BOS token embedding 408a. In a parallel processing engine (e.g., combining N-flank processing engine 138 and C-flank processing engine 140), the N-flank and C-flank sequences can be appended together with a single BOS token to create a combined flank sequence 402b. After flank sequence representation 404b is generated by the embedding module, a transformed flank sequence representation 406b is generated by processing subsystem 304, including transformed BOS token embedding 408b. As shown, transformed BOS token 408a is first combined with the transformed BOS token embedding 408b, the combined result is then combined with transformed BOS token 408c and the transformed BOS token embedding 408d. In each of the two combination operations, element-wise addition, element-wise multiplication, concatenation, or dot-product multiplication may be used.

In parallel, in MHC processing engine 141 in FIG. 1, MHC sequence 402c (e.g., MHC sequence 135 of IPC sequence 124, corresponding to an allele for MHC Class I, or to an allotype for MHC Class II) as appended with a BOS token is used to generate an MHC sequence representation 404c. During the transformation stages, transformed MHC sequence representation 406c is then generated, including transformed BOS token embedding 408c. Also in parallel, a BOS token-appended TCR sequence 402d (e.g., TCR sequence 131 of IPC sequence 124) can be used to generate TCR sequence representation 404d, from which transformed TCR sequence representation 406d is generated, including transformed BOS token embedding 408d.

The transformed BOS token embeddings may then be combined together by composite subsystem 306 in FIG. 3 (e.g., by concatenating 408a and 408b and applying element-wise multiplication to the concatenated 408a+408b, 408c, and 408d) and the product vector passed through a multilayer perceptron network (e.g., output subsystem 310) in order to provide a binary prediction of binding affinity, presentation likelihood, immunogenicity prediction, or another downstream evaluation.

FIG. 4B illustrates an example workflow 420 for obtaining one or more interaction predictions, one or more interaction affinity predictions, and/or one or more immunogenicity predictions, in accordance with some embodiments. Similar to the workflow 400 in FIG. 4A, a tokenizer (not depicted) can create a tokenized sequence comprising a BOS token-appended peptide sequence 402a and a BOS token-appended nFlank+cFlank sequence 402b. An embedding module (not depicted) can generate (e.g., via representation subsystem 302 in FIG. 3) a BOS+peptide sequence representation 404a (which may correspond to 312 in FIG. 3) based on the BOS token-appended peptide sequence 402a and a BOS+nFlank+cFlank sequence representation 404b that represents a combined version of BOS+N-flank sequence representation (which may correspond to 324 in FIG. 3) and BOS+C-flank sequence representation (which may correspond to 330 in FIG. 3) based on the BOS token-appended nFlank+cFlank sequence 402b. One or more transformer stages of the system (e.g., in processing subsystem 304) can then create transformed BOS+peptide sequence representations 406a and a transformed BOS+nFlank+cFlank sequence representation 406b. During the transformer stages, the system can further generate BOS token embeddings as part of transformed sequence representations 406a and 406b, including BOS token embedding 408a of transformed BOS+peptide sequence representations 406a and BOS token embedding 408b of transformed BOS+nFlank+cFlank sequence representation 406b.

The workflow 400 in FIG. 4A does not incorporate processing of protein data (e.g., source protein 123 in FIG. 1) from which the peptide (e.g., peptide 118 in FIG. 1 that can be represented by 402a) and the N-flank/C-flank (e.g., N-flank 120 and C-flank 122 that can be represented by 402b) are identified. As discussed above with reference to FIG. 1, the protein 123 is a source protein for the amino acid chain 116 (including the peptide 118 in FIG. 1, N-flank 120 in FIG. 1, C-flank 122 in FIG. 1), which can be generated through proteolysis, which is the process by which proteins (e.g., the protein 123) are broken down into smaller polypeptides or amino acids. The protein 123 can be broken down into smaller polypeptides or amino acids by enzymatic cleavage, where specific enzymes called proteases cut the peptide bonds between amino acids in the protein 123. In contrast, the workflow 420 in FIG. 4B can further include a protein processing engine (e.g., the protein processing engine 162 in FIG. 1) to generate and process a protein sequence embedding 422 encapsulating information about the protein. As shown in FIG. 4B, a dimensionality reduction module 424 can receive a protein sequence embedding 422 to reduce the dimensionality of the protein sequence embedding 422 and generate an output vector. As discussed below with reference to FIG. 4E, the proteins sequence embedding 422 can be generated by a Protein Language Model (PLM) based on a protein sequence (e.g., protein sequence 160 in FIG. 1).

In some examples, the dimensionality reduction module 424 can be implemented as a fully connected layer (“FCN”). An FCN can include a neural network in which each neuron applies a transformation (e.g., linear transformation) to the input vector through a weight matrix. As a result, all possible connections layer-to-layer are present and thus every input of the input vector (i.e., the protein sequence embedding 422) influences every output of the output vector. In addition to reducing the dimensionality of the input vector, the FCN can include a relatively large number of learnable parameters, allowing the neural network to encode useful information into the output vector. The output vector (i.e., the dimensionally reduced version of the protein sequence embedding 422) can be further aggregated with 408a and 408b. The dimensionality reduction module 424 can encode the mapping from protein feature to useful features such as the cellular compartmentalization and gene expression, among other things. While FIG. 4B shows element-wise addition is used to aggregated 408a, 408b, and the output vector of the dimensionality reduction module 424, other integration methods can be used, such element-wise averaging, element-wise multiplication, concatenation, and dot-product multiplication.

The incorporation of the protein information advantageously allows the incorporation of useful protein features. The workflow 420 can select peptides that are more likely to be presented by MHC molecules because the workflow can capture features such as: processing signals (i.e., flank signals produced when enzymes break down proteins into peptides), expression of the gene associated with the source protein, cellular localization of the source protein, and other suitable features. For example, for MHC I, it is preferred that the source protein originated within the cell, and for MHC II, vesicle or extracellular proteins are preferred. In sum, there are many features related to the source protein that affect peptide presentation, which are advantageously incorporated in the workflow 420. Additionally, the use of PLM can be advantageous relative to the use of transformers. A typical transformer can have a large number of parameters. Thus, training a transformer using a relatively small training dataset can cause overfitting. In contrast, the use of PLM can allow the workflow to learn useful generalizable features, thus reducing the likelihood of overfitting and improving the performance of the workflow.

In some embodiments, the system can enable or disable the incorporation of the processing of the protein data (i.e., the protein processing engine 162 in FIG. 1) depending on the use case. For example, when the system is used to develop personalized cancer vaccines and the subject produces the peptides in a similar way that the peptides in the presentation data set are produced, the protein processing engine can be enabled. In contrast, when the system is used to develop antibody drugs, the protein processing engine can be disabled when the PLM provides information about endogenous proteins and antibodies drugs are not endogenous.

The workflow 420 is also different from the workflow 400 in FIG. 4A in the processing of MHC data. As discussed above, the workflow 400 involves using a BOS token-appended MHC sequence 402c to generate a BOS+MHC sequence representations 404c and then using one or more transformer stages to generate a transformed BOS+MHC sequence representation. The workflow 400 further involves obtaining a BOS token embedding 408c and aggregating the BOS token embedding 408c with the rest of the BOS embedding tokens. In contrast, in the workflow 420 in FIG. 4B, a dimensionality reduction module 428 can receive an MHC sequence embedding 426 to reduce the dimensionality of the MHC sequence embedding 426 and generate an output vector. As discussed below with reference to FIG. 4F, the MHC sequence embedding 426 may be generated using a PLM based on an MHC sequence (e.g., MHC sequence 135 in FIG. 1).

The goal of the dimensionality reduction module 428 is to reduce the number of parameters used to represent information about the MHC sequence. The dimensionality reduction module 428 removes or de-prioritizes less useful parameters, such as parameters that do not vary from one MHC allele to another (e.g., with variances below a certain threshold). For example, similar binding behaviors can be removed or de-prioritized, while dissimilar binding behaviors can be preserved. In some embodiments, the dimensionality reduction module 428 can be implemented as a principal component analysis (PCA) model. The PCA model can be configured to receive the MHC sequence embedding 426 having N vector values and generate a dimensionality reduced version of the MHC sequence embedding 426 having M vector values (M<N). In some embodiments, to configure the PCA model, the PCA model first receives a plurality of MHC vectors (e.g., each having N vector values corresponding to N features) corresponding a plurality of MHC sequences and ranks the N features based on how each feature varies across the plurality of MHC vectors (e.g., based on the variance associated with each feature). After the PCA model determines the ranking of the N features, the PCA model can then receive an input MHC sequence embedding 426 having N vector values, re-order the N vector values in the MHC sequence embedding 426 according to the ranking of the N features, and generate a dimensionality reduced version of the MHC sequence embedding 426 having M vector values (M<N), for example, by preserving only the first M vector values of the re-ordered N vector values. It should be appreciated that the dimensionality reduction module 428 can use other dimensionality reduction methods, such as UMAP, t-distributed stochastic neighbor embedding, independent component analysis, multidimensional scaling, Isomap, and deep-learning-based dimensionality reduction techniques.

The use of the dimensionality reduction module 428 to further process the MHC sequence embedding 426 provides a number of technical advantages. For example, if a different type of model such as a neural network is trained to reduce the dimensionality of an input embedding, overfitting may occur. Overfitting is an undesirable machine learning behavior that occurs when the machine learning model gives accurate outputs for training data but not for new data. If the neural network is trained using the same set of alleles used to train the PLM model, which typically is a relatively small set of alleles, the neural network model may produce a correct output when processing an input embedding corresponding to an allele in the training data but produce an incorrect output when processing an input embedding corresponding to a new allele not included in the training data. The PCA technique, on the other hand, can be trained using a larger set of alleles (e.g., the space of all alleles) and thus can efficiently and accurately reduce the dimension of an input embedding for a new allele not in the training dataset, thus avoiding the overfitting problem.

With reference to FIG. 4B, the output of the dimensionality reduction module 428 (i.e., the dimensionality reduced version of the MHC sequence embedding 426) can optionally be provided to an FCN module 429. The FCN module 429 can include a neural network in which each neuron applies a transformation (e.g., linear transformation) to the input vector through a weight matrix. As a result, all possible connections layer-to-layer are present and thus every input of the input vector (i.e., the protein sequence embedding 422) influences every output of the output vector. The FCN module 429 can further reduce the dimensionality of the output vector of the dimensionality reduction module 428. In addition, the FCN module 429 can encode the relationship between MHC sequence and the types of peptides that the MHC sequence presents. Additionally, the FCN module 429 can encode information useful for the downstream processing. For example, two MHC sequence embeddings that are represented by similar sequences may be close to each other in the latent space due to similarity in the sequence data, but the two MHCs may in fact function differently (e.g., presenting different peptides). The FCN module 429 can adjust for the discrepancy in the latent space such that the output vector is more useful for downstream processing (e.g., because the FCN module 429 allows encoding of information about how similar peptides and similar MHC present to one another). The output vector of the FCN module 429 can be aggregated with the output 410 as shown in FIG. 4B. While FIG. 4B shows element-wise multiplication is used, other integration methods can be used, such element-wise averaging, element-wise addition, concatenation, and dot-product multiplication.

FIG. 4C depicts an example workflow 470 for predicting a peptide interaction with one or more MHC molecules expressed by one or more alleles or allotypes, in accordance with some embodiments. Similar to the workflow 400 in FIG. 4A, a tokenizer (not depicted) can create a BOS token-appended nFlank+cFlank sequence 402b. An embedding module (not depicted) can generate (e.g., via representation subsystem 302 in FIG. 3) a BOS+nFlank+cFlank sequence representation 404b based on the BOS token-appended nFlank+cFlank sequence 402b. One or more transformer stages of the system (e.g., in processing subsystem 304 in FIG. 3) can then create a transformed BOS+nFlank+cFlank sequence representation 406b. During the transformer stages, the system can further generate a BOS token embedding 408b of transformed BOS+nFlank+cFlank sequence representation 406b.

Unlike the workflow 400 in FIG. 4A, the workflow 470 does not receive a BOS token-appended peptide sequence. Instead, the workflow 470 receive a peptide sequence (e.g., peptide sequence 126 in FIG. 1) that is not appended with a BOS token and create a tokenized peptide sequence 402e based on the peptide sequence. The embedding module can generate (e.g., via representation subsystem 302 in FIG. 3) a peptide sequence representation 404e based on the tokenized peptide sequence 402e. One or more transformer stages of the system (e.g., in processing subsystem 304 in FIG. 3) can then create transformed peptide sequence representations 406e.

Further, the workflow 470 receives a BOS vector embedding 472 (which can be generated using a PLM model based on a BOS vector) and an MHC sequence embedding 426. The BOS vector embedding 472 can be a random vector selected at training time, for example, an intercept representing one or more random bias terms. The dimensionality reduction component 428 can receive the MHC sequence embedding 426 and obtain an output vector as discussed above with reference to FIG. 4B. The output vector of the dimensionality reduction component 428 (e.g., with parameters with lower variances removed or de-prioritized) can be provided to an FCN module 473, which can further reduce the dimensionality of the vector. At aggregator 471, the workflow 470 aggregates the BOS vector embedding 472 and the output vector of the FCN module 473. While FIG. 4C shows element-wise addition at the aggregator 471, other integration methods can be used, such element-wise averaging, element-wise multiplication, concatenation, and dot-product multiplication. Accordingly output vector of the aggregator 471 encodes information from the BOS vector and the MHC sequence.

The workflow 470 further includes a cross-attention module 474. The cross-attention module 474 can be implemented as a self-attention transformer having three components: Query (Q), Key (K), and Value (V). As shown in FIG. 4C, both the K component of the cross-attention module 474 and the V component of the cross-attention module 474 come from the transformed peptide sequence representations 406e (i.e., implementing a self-attention mechanism). The Q component of the cross-attention module 474 comes from the output vector of the aggregator 471 (i.e., the combined vector of the BOS vector embedding and the MHC embedding). The cross-attention module 474 can perform an attention function, which involves mapping a query and a set of key-value pairs to an output, where the query, keys, values, and output are all vectors. The output is computed as a weighted sum of the values, where the weight assigned to each value is computed by a compatibility function of the query with the corresponding key. Specifically, the cross-attention module 474 can perform scaled dot-product attention or other suitable type of integration technique. Additional details of the scaled dot-product attention can be found in Ashish Vaswani et al., Attention is All You Need, Neural Information Processing Systems (2017). The configuration of the workflow 470 is advantageous because it incorporates allele-specific binding information. Allele information can be valuable when processing the peptide data. For example, a peptide may contain multiple binding cores, which may bind to different alleles. The workflow 470 uses the cross-attention module 474 to incorporate an allele-specific vector (i.e., component Q) into the processing of the peptide data. This way, the workflow can capture the binding core information from peptides that have multiple binding cores that can bind to different alleles and produce multiple binding core predictions corresponding to the multiple different alleles. In some examples, a peptide can have more than one element focus score, each element focus score corresponding to a different allele.

FIG. 4D illustrates an alternative embodiment 476 of the workflow 470. As shown, the workflow 476 eliminates the processing of the BOS vector embedding 477 in the workflow 470 and also eliminates the aggregator 475 because the allele information (i.e., the MHC sequence embedding) is already considered by the cross-attention module 474. The workflow 476 shares similar technical advantages as the workflow 470 but can be more computationally efficient due to the elimination of modules. Further, the workflow 476 in FIG. 4D can provide a latent space that is dependent on the allele.

FIG. 4E depicts an example process for generating a protein sequence embedding, in accordance with some embodiments. With reference to FIG. 4E, a PLM 444 can receive a protein sequence 160 and output a protein sequence embedding 422. PLMs are machine-learning models (e.g., deep-learning models) that can be based on natural language processing methods such as attention and transformers and can be trained on ensembles of protein sequences. Protein language models are trained to understand and predict the properties of proteins based on the amino acid sequence forming such a protein. In some instances, protein language models can infer a range of characteristics from amino acid sequences, including primary, secondary, tertiary, and quaternary structures. PLMs predict how proteins fold, their domains, active sites, and stability. PLMs can also forecast protein-protein and protein-nucleic acid interactions, post-translational modifications, and the effects of mutations. PLMs can identify localization signals within the cell, understand evolutionary relationships, and predict protein function. Likewise, PLMs can provide insights into the dynamic behavior of proteins and identify potential drug binding sites, valuable for drug discovery and understanding the molecular basis of diseases. In some embodiments, the PLM 444 can comprise an Evolutionary Scale Modeling (ESM model) or a variation of the ESM model. In some embodiments, the PLM 444 can comprise ProteinBERT, UniRep, or other suitable type of PLM.

In some embodiments, the PLM 444 comprises a pretrained protein language model such as a pretrained ESM model. In some embodiments, the input protein sequence 160 can include a sequence of amino acid residues and the PLM 444 can be configured to obtain a plurality of embeddings (i.e., vector representations) by obtaining, for each amino acid residue, a corresponding embedding. The model can be further configured to obtain a single embedding 422 by aggregating the plurality of embeddings corresponding to the sequence of amino acid residues (e.g., by element-wise averaging).

FIG. 4F depicts an example process for generating a MHC sequence embedding, in accordance with some embodiments. With reference to FIG. 4F, a PLM 445 can receive an MHC sequence 135 and output an MHC sequence embedding 426. As discussed herein, PLM is trained using a large number of proteins and thus can encode useful information and context about the input sequence represented by the sequence embedding. In some embodiments, the PLM 445 can comprise an Evolutionary Scale Modeling (ESM model) or a variation of the ESM model. In some embodiments, the PLM 445 can comprise ProteinBERT, UniRep, or the like. In some embodiments, the PLM 445 comprises a pretrained protein language model such as a pretrained ESM model. In some embodiments, the MHC sequence 135 can include a plurality of amino acids that compose the corresponding allele. The PLM 445 can be configured to obtain a plurality of embeddings (i.e., vector representations) by obtaining, for each amino acid, a corresponding embedding. The model can be further configured to obtain a single embedding 426 by aggregating the plurality of embeddings corresponding to the sequence of amino acid (e.g., by element-wise averaging).

FIG. 4G is an example workflow diagram 450 for more efficiently predicting peptide interactions with an MHC molecule that may have been expressed by multiple alleles or allotypes, in accordance with some embodiments. This technique can be applied in any of the workflows described herein. This technique can advantageously predict peptide interactions with an MHC molecule when binding or elution likelihood of a peptide is known, but data was collected for multiple alleles or allotypes, and it is unknown exactly which of the multiple alleles or allotypes bound to the peptide. As shown in FIG. 4G, a set of alleles/allotypes 452 (e.g., HLA 1, HLA 2, HLA 3, and HLA 4) for which data was collected have been tokenized and embedded (e.g., using representation subsystem 302) to create one MHC sequence representation 404c per allele/allotype. Since the data for each sample for each allele/allotype can be sparse, steps can be taken (e.g., by representation subsystem 302) to compress the MHC sequence representations. The MHC sequence representations for the detected binding interaction can be flattened into a single array 454, from which empty rows can be removed, thereby resulting in a dense, combined MHC sequence representation 456 that is then processed as usual by processing subsystem 304 to generate binding affinity predictions 458 (e.g., during both the model training and inference phases). Once the predictions 458 have been generated, the model output can be re-sparsified (e.g., embeddings 460) as needed for downstream tasks.

FIG. 4H is an example workflow 480 for attention masking at each transformer stage of transformed BOS+peptide sequence representations 406a, in accordance with some embodiments. This technique can be applied in any of the workflows described herein involving processing of BOS-token appended peptide sequences. The transformer stages (e.g., as executed by processing subsystem 314 and processing sub-blocks 542, 594b, and 602) store pairwise information in attention maps. In order to force the model to only pay attention to peptide binding core sequences having a specified length (e.g., nine amino acids long, as shown in attention mask 482, or some other core length), masks 482 and 484 are applied to the attention maps in order to restrict the range of sequential amino acids (e.g., considering at most nine positions ahead in the sequence, or some other core length) over which the model can record information about pairwise correlations between amino acids. In a final transformer stage, final attention map 484 may restrict the model to only attending to the BOS token 408a of transformed BOS+peptide sequence representations 406a and recording the maximum value as the start of the binding core.

FIG. 4I illustrates example attention maps used in transformer stages, in accordance with some embodiments. Each attention map visualizes the attention weights assigned by a transformer stage to different parts of the input. In FIG. 4I, the attention maps are represented as heatmaps in which a lighter color corresponds to a higher attention weight. Specifically, FIG. 4I illustrates an example attention map 483 used in a transformer stage to obtain the transformed BOS+peptide sequence representation 406a for a given peptide and an example attention map 485 applied to obtain the BOS token 408a for the given peptide. As discussed above with reference to FIG. 4H, an attention mask 482 has been applied to the attention map 483 to force the model to only pay attention to peptide binding core sequences having a specified length (e.g., nine amino acids long, as shown in attention mask 482, or some other core length) and an attention mask 484 has been applied to the attention map 485.

In general, the position zero (i.e., the start position) of a peptide is unlikely to be the binding core start position because the binding core is expected to be a portion (e.g., a 9-mer) of the peptide (e.g., a 20-mer) and to be surrounded by binding core flanks within the peptide. However, the attention mechanism in the workflow 480 may result in the over-prediction of the position zero in a peptide as the binding core start position for certain datasets and/or certain workflows. To address the over-prediction problem, the workflow 480 can include a calibration step to remove the bias toward the position zero. Before performing the calibration step, the system (e.g., computing platform 102 in FIG. 1) can first obtain a set of random peptides with a uniform length distribution. For each given peptide length, the system can calculate the average attention value (e.g., from the transformed BOS+peptide sequence representations 406a in FIGS. 4A-B, the cross-attention module 474 in FIGS. 4C-4D) at each position (e.g., position zero, position one, position two, etc.) across the set of random peptides. In other words, the system can calculate, for each given peptide length N, the average attention values for the various positions 0 to N−1 (i.e., the average attention value for position zero, the average attention value for position one, . . . , and the average attention value for position N−1). To the extent that these average attention values differ, the average attention values represent the model bias because the probability of binding core starting at any position should be equal and thus the attention values should be uniformly distributed (i.e., the average attention values should be identical).

Returning to FIG. 4I, in workflow 480, the system can perform the calibration step by modifying the attention map 485. For the given peptide with a length N, the system can subtract the average attention values for the N positions (i.e., the average attention value for position zero, the average attention value for position one, . . . , and the average attention value for position N−1) from the N positions in the attention map 485. After the subtraction, the modified attention map 485 can be applied to obtain the BOS token 408a for the given peptide in the workflow 480. As discussed above, the average attention values represent the model bias and, by subtracting the average attention values from the attention map, the calibration step removes model biases toward any single position in the peptide.

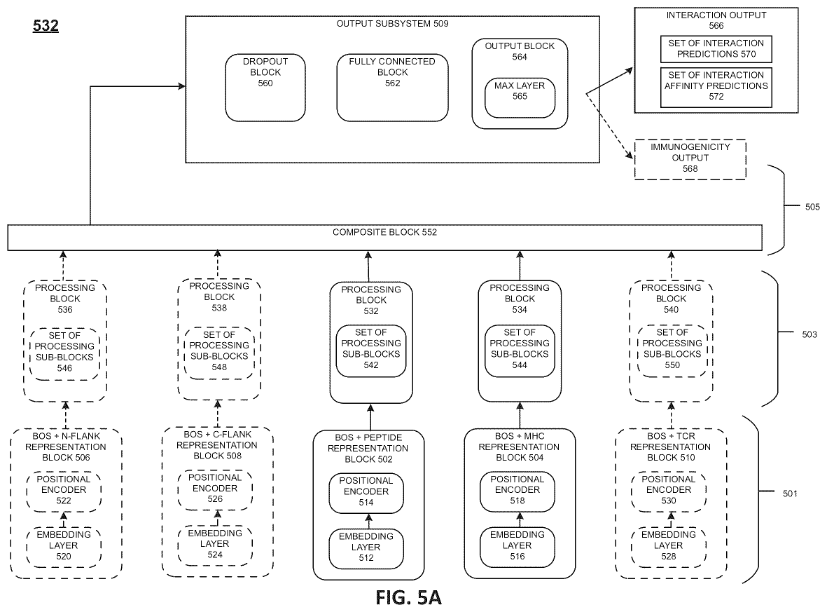

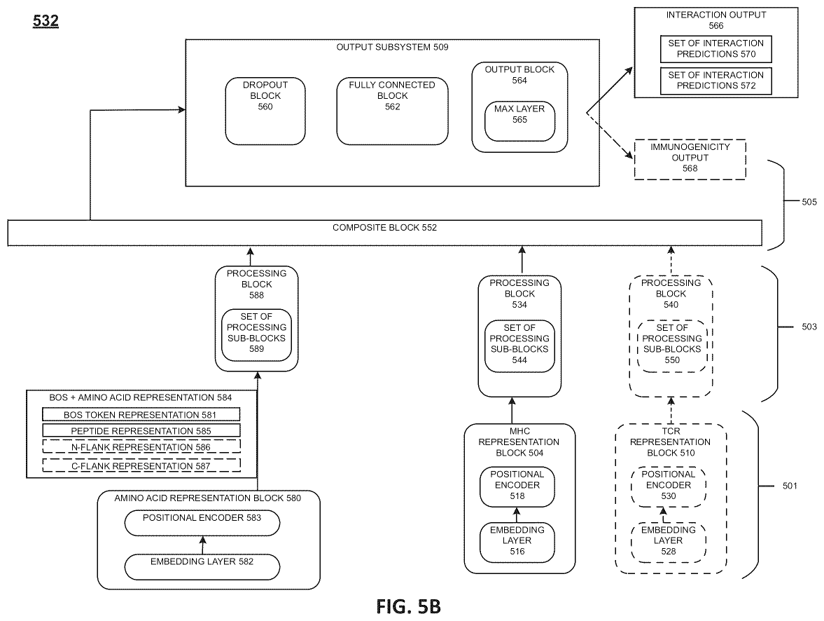

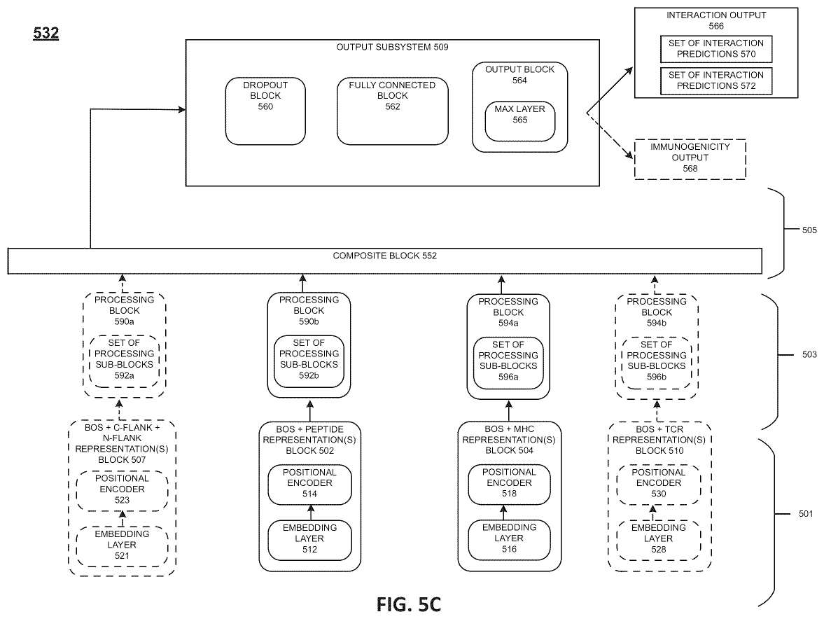

FIGS. 5A-5C are schematic diagrams of different configurations for a machine-learning model 532, in accordance with some embodiments. Machine-learning model 532 is one example of an implementation for machine-learning model 132 in FIGS. 1 and 3 and the workflow 400 in FIG. 4A. The machine-learning model 532 can be any type of machine-learning model including, but not limited to, an attention-based machine-learning model. As shown in the schematic diagram of FIG. 5A, the machine-learning model 532 includes representation subsystem 501 (e.g., an embedding module that generates sequence representations 404), processing subsystem 503 (e.g., one or more transformer stages that generate transformed sequence representations 406), composite subsystem 505 (e.g., that combines the BOS token embeddings 408 of transformed sequence representations 406), and output subsystem 509, which are examples of implementations for representation subsystem 302, processing subsystem 304, composite subsystem 306, and output subsystem 310, respectively, in FIG. 3.

Representation subsystem 501 may include a BOS+peptide representation block 502 and a BOS+MHC representation block 504. In some embodiments, the representation subsystem 501 further includes a BOS+N-flank representation block 506, a BOS+C-flank representation block 508, or both. In some embodiments, the representation subsystem 501 further includes a BOS+TCR representation block 510. One or more (e.g., each) representation blocks include at least one embedding layer (e.g., embedding layer 512, embedding layer 516, embedding layer 520, embedding layer 524, or embedding layer 528) and may include, for example, at least one positional encoder (e.g., positional encoder 514, positional encoder 518, positional encoder 522, positional encoder 526, or positional encoder 530).

An embedding layer may embed a sequence by, for example, transforming an initial non-numeric sequence representation (e.g., a string of amino acid identifiers) into a numeric sequence representation to generate an embedded representation. In some embodiments, an embedded amino acid sequence representation indicates, for each position of a sequence and for each of a set of (e.g., 21) amino acids, whether the particular amino acid is present at the position. The embedding can be performed using, for example, one-hot encoding, evolutionarily-motivated encodings such as BLOSUM, randomly or pseudo-randomly initialized learned embeddings, or a combination thereof. The embedded representation can be positionally encoded to generate an encoded representation. The representation produced by a representation block can be the encoded representation or an aggregation (e.g., concatenation or sum) of the encoded representation and the embedded representation.

In some cases, the order of values in an input data set can be useful. Positional encoders can be used and added to the embedded representation, with the positional encoding using an encoding algorithm that is learned or fixed. For example, a fixed positional encoding can be determined using a sine and/or cosine function (e.g., having an intra-sequence position and/or a dimension as the independent variables). The positional encoding may have the same dimension as the encoded representation. The positional encodings can be summed with the embedded representation to produce a position-indicative embedded representation of the sequence that is fed into the processing subsystem 503.

For example, the BOS+peptide representation block 502 may include an embedding layer 512 and a positional encoder 514. The embedding layer 512 embeds a peptide sequence (e.g., peptide sequence 126 in FIG. 1) to generate an embedded peptide representation, and the positional encoder 514 positionally encodes the embedded peptide representation to generate a peptide sequence representation (e.g., peptide sequence representation 312 in FIG. 3). The BOS+N-flank representation block 506 may include an embedding layer 520 and a positional encoder 522. The embedding layer 520 embeds an N-flank sequence (e.g., N-flank sequence 128 in FIG. 1) to generate an embedded N-flank representation, and the positional encoder 522 positionally encodes the embedded N-flank representation to generate an N-flank sequence representation (e.g., N-flank sequence representation 324 in FIG. 3). The BOS+C-flank representation block 508 may include an embedding layer 524 and a positional encoder 526. The embedding layer 524 embeds a C-flank sequence (e.g., C-flank sequence 130 in FIG. 1) to generate an embedded C-flank representation, and the positional encoder 526 positionally encodes the embedded C-flank representation to generate a C-flank sequence representation (e.g., C-flank sequence representation 330 in FIG. 3).

BOS+MHC representation block 504 may include an embedding layer 516 and a positional encoder 518. The embedding layer 516 embeds an MHC sequence (e.g., MHC sequence 135 in FIG. 1) to generate an embedded MHC representation, and a positional encoder 518 positionally encodes the embedded MHC representation to generate an MHC sequence representation (e.g., MHC sequence representation 318 in FIG. 3). BOS+TCR representation block 510 may include an embedding layer 528 and a positional encoder 530. The embedding layer 528 embeds a TCR sequence (e.g., TCR sequence 131 in FIG. 1) to generate an embedded TCR representation, and a positional encoder 530 positionally encodes the embedded TCR representation to generate a TCR sequence representation (e.g., TCR sequence representation 336 in FIG. 3).

The sequence representations generated by the representation subsystem 501 are sent as input into the processing subsystem 503 for processing. In some embodiments, the sequence representations input into the processing subsystem 503 may include embeddings corresponding to appended BOS tokens. For example, a peptide sequence representation may include an embedding for a BOS token appended to the peptide sequence (e.g., BOS+peptide sequence representation 308 in FIG. 3), and an MHC sequence representation may include an embedding for a BOS token appended to the MHC sequence (e.g., BOS+MHC sequence BOS representation 318 in FIG. 3).

The processing subsystem 503 may include various mechanisms that determine, for each of one or more (e.g., all) positions in a sequence representation, an element-focused score. An element-focused score may indicate the level of attention or importance. For example, the element-focused scores of a set of amino acid sequence representations may indicate where the binding core of a peptide begins. An element-focused score can then be used to generate a transformed value for a position.

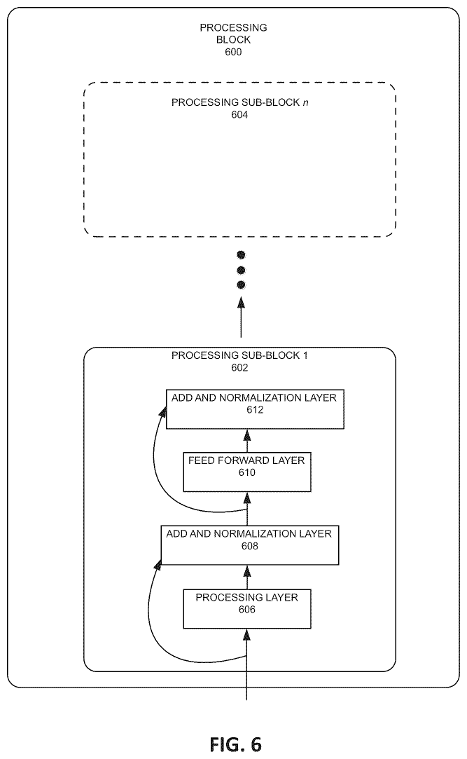

Processing subsystem 503 includes a processing block 532 and a processing block 534. In some embodiments, the processing subsystem 501 may include a processing block 536, processing block 538, processing block 540, or a combination thereof. The processing block 532 receives a peptide sequence representation from the BOS+peptide representation block 502 and processes the peptide sequence representation using a set of processing sub-blocks 542 to generate a transformed peptide sequence representation (e.g., transformed peptide sequence representation 316 in FIG. 3). The transformed amino acid sequence representation can be generated based on an amino acid sequence representation and one or more element-focused scores (representing binding cores of the set of amino acid sequence representations). One example implementation for a processing sub-block that executes one or more transformer stages to generate transformed sequence representations is described below in greater detail in the context of FIG. 6. The processing block 534 receives an MHC sequence representation from the BOS+MHC representation block 504 and processes the MHC sequence representation using a set of processing sub-blocks 544 to generate a transformed MHC sequence representation (e.g., transformed MHC sequence representation 322 in FIG. 3). The transformed MHC sequence representation can be generated based on the MHC sequence representation and one or more element-focused scores. In some embodiments, the element-focused scores used to generate a transformed amino acid sequence representation can be different from the element-focused scores used to generate a transformed MHC sequence representation.

Further, when included, the processing block 536 receives an N-flank sequence representation from the BOS+N-flank representation block 506 and processes the N-flank sequence representation using a set of processing sub-blocks 546 to generate a transformed N-flank sequence representation (e.g., transformed N-flank sequence representation 328 in FIG. 3). In some embodiments, the processing block 538 receives a C-flank sequence representation from the BOS+C-flank representation block 508 and processes the C-flank sequence representation using a set of processing sub-blocks 548 to generate a transformed C-flank sequence representation (e.g., transformed C-flank sequence representation 334 in FIG. 3). In some embodiments, the processing block 540 receives a TCR sequence representation from the BOS+TCR representation block 510 and processes the TCR sequence representation using a set of processing sub-blocks 550 to generate a transformed TCR sequence representation (e.g., transformed TCR sequence representation 340 in FIG. 3).

In some embodiments, one or more processing sub-blocks may separately process representations of different parts, or all of the amino acid sequence and/or IPC sequence. In some embodiments, one or more sequence representations (e.g., N-flank sequence representation, C-flank sequence representation, peptide sequence representation, MHC sequence representation, TCR sequence representation) can be processed separately in different iterations of the processing sub-blocks. For example, an encoded representation of an amino acid sequence may include a feature vector representing the amino acid, and encoded representations of the sequences (e.g., all or part of the amino acid sequence, all or part of the IPC sequence) can then be concatenated and fed to another iteration of the processing sub-block.

As shown in FIG. 5A, an amino acid sequence can be processed separately and independently from the processing of an IPC sequence. By having separate and independent processing engines for the peptide sequence(s), N-flank sequence(s), C-flank sequence(s), MHC sequence(s), and/or TCR sequence(s) prior to the composite subsystem 505, the predictive performance of the machine-learning model 532 can be enhanced. For example, generating the transformed peptide sequence representation using the BOS+peptide representation block 502 and the processing block 532 along a path that is separate from the generation of the transformed IPC sequence representation and doing so prior to generating the composite representation increases the accuracy of the output generated by the output subsystem 509. Similarly, the predictive performance (e.g., accuracy) of the machine-learning model 532 can be enhanced by generating the transformed N-flank sequence representation using the BOS+N-flank representation block 506 and the processing block 536 along a separate path, the transformed C-flank sequence representation using the BOS+C-flank representation block 508 and the processing block 538 along a separate path, the transformed MHC sequence representation using the BOS+MHC representation block 504 and the processing block 534 using a separate path, the transformed TCR sequence representation using the BOS+TCR representation block 510 and the processing block 540 using a separate path, or a combination thereof. In some embodiments, separate paths may enable efficient processing (e.g., using reduced computing resources, quicker processing, etc.) because multiple amino acid-IPC (peptide-MHC, peptide-TCR) combinations can be considered in a modular way and/or processed in parallel.

The transformed sequence representations output from the processing subsystem 503 are sent into composite subsystem 505 for processing. The composite subsystem 505 includes a composite block 552. The composite block 552 may form one or more composite representations (e.g., composite representation(s) 342 in FIG. 3) using the transformed representations output from the processing subsystem 503. For example, the composite block 552 may multiply sets of transformed sequence representations (e.g., set of amino acid sequence representations, set of IPC sequence representations) to form composite representations.

In some embodiments, the size of the output generated by the composite block 552 can be equal to, for example, m×n, where m is equal to the total number of amino acids being considered plus 1 (e.g., for the BOS token) plus any padding to conform to the normalized sequence length (e.g., 39), and n is equal to a number of features (a predetermined value, e.g., 600). A single column (having n values) can be selected for further processing as an output of the composite block 552. The single column can be a first column and/or a column associated with the BOS token. In some embodiments, the output from the composite block 552 can be aggregated to form a single vector, which may then be fed into the output subsystem 509.

Output subsystem 509 may include various blocks, sub-blocks, layers, or a combination thereof for generating a final output. In some embodiments, the output subsystem 509 includes a dropout block 560, a fully connected block 562, and an output block 564. The dropout block 560 may include, for example, one or more dropout layers. The fully connected block 562 may include, for example, one or more fully connected layers. The output block 564 may include, for example, one or more layers for filtering, selecting, transforming, or otherwise generating a result. For example, the output block 564 may include at least one max layer 565 configured to select a subset of the inputs received by the output block 564 based on, for example, selected thresholds or ranges.

In some cases, a composite representation is received and processed by the dropout block 560 to generate a first output that is received by the fully connected block 562. The fully connected block 562 may receive and process this first output to generate a second output, at least a portion of which is received by the output block 564. The output block 564 receives and processes its input to generate a result, such as an interaction output 566, an immunogenicity output 568, or both.

In some embodiments, the fully connected block 562 can be configured to generate one or more outputs having a dimensionality that is smaller than the dimensionality of its inputs (fed into the fully connected block 562, e.g., smaller than the predetermined number of features). For example, an output of the fully connected block 562 may include a single value, two values, or three values, each corresponding to a prediction pertaining to a target interaction or immune response. The fully connected block 562 may include, for example, a single hidden layer, two hidden layers, or three or more hidden layers. A number of nodes in an initial hidden layer can be larger than a number of nodes in a subsequent hidden layer. For example, a first hidden layer can include 256 nodes, while a second hidden layer can include 126 nodes. In some embodiments, each output from the fully connected block 562 may include a real number score, which may, for example, be converted to a binary and/or categorical result (e.g., using a trained activation function) and/or converted into a scaled number. For example, the scaled number may include a probability on a scale from 0 to 1.

Interaction output 566 may include, for example, one or more of: a set of interaction predictions 570 or a set of interaction affinity predictions 572, with respect to one or more target interactions. An interaction prediction 570 may include, for example, a prediction for a corresponding amino acid-IPC combination, such as a peptide-IPC (e.g., peptide-MHC, peptide-TCR) combination, of whether or the extent to which the IPC (e.g., MHC, TCR) will bind to the peptide. In some embodiments, an interaction prediction 570 may include, for example, a prediction for a corresponding peptide-IPC (e.g., peptide-MHC) combination of whether the IPC (e.g., MHC) will bind to the peptide. In some embodiments, an interaction affinity prediction 570 may include, for example, a prediction of an affinity for a target interaction for a corresponding peptide-IPC (e.g., peptide-MHC, peptide-TCR) combination. The target interaction can be, for example, the binding of the peptide and the IPC. The affinity for the target interaction, which can be, for example, a binding affinity, indicates the strength, tendency, and/or stability of the binding between the peptide and the IPC.

Immunogenicity output 568 comprises a set of immunogenicity predictions. An immunogenicity prediction may include, for example, a prediction of immunogenicity with respect to a corresponding amino acid-IPC combination. For example, an immunogenicity prediction may indicate the ability of the peptide to provoke an immune response with respect to the particular IPC of interest (e.g., MHC, TCR). In some embodiments, the predicted amino acid-IPC interaction comprises a prediction of tumor-specific immunogenicity of a peptide. In some embodiments, the predicted amino acid-IPC interaction identifies a subset of peptide sequences having increased tumor-specific immunogenicity or increased likelihood of presentation by the IPC relative to a set of peptide sequences.

In some cases, a first portion of the output from the fully connected block 562 is sent into the output block 564, while a second portion of the output from the fully connected block 562 is in its final form and used as a set of interaction affinity predictions 572.

In some embodiments, a transformed composite representation received at the output subsystem 509 and processed by the fully connected block 562. The fully connected block 562 may process the transformed composite representation to generate a first output that is sent into the dropout block 560. The output of dropout block 560 or a portion thereof may then be sent to the output block 564 for processing.

In some cases, each fully connected sub-block within the fully connected block 562 may have dropout applied, followed by a batch normalization layer. In some embodiments, the output block 564 is used for deconvolution such that amino acid-IPC interactions (e.g., twelve paired peptide-MHCII interactions or six paired peptide-MHCI interactions) correspond to a single selected MHC II allotype or MHC I allele (respectively) by applying an activation function (e.g., via max layer 565 which may include a softmax function or just using the maximum value) on the presentation predictions. During training, the selected peptide-MHC interaction output can be normalized as a value between 0 and 1 and can be compared to a true presentation value using a loss function (e.g., binary loss function) to generate an error for tuning the model parameters.

In some embodiments, the output from the output subsystem 509 may include multiple results that include, for each IPC (e.g., MHC) allele, a predicted amino acid-IPC interaction that indicates whether and/or a probability that the peptide binds to the IPC allele. The allele-specific predictions can be output, or in some cases, the max layer 565 can be used to determine the maximum of the allele-specific predictions, and the maximum can be output.

In this manner, the output subsystem 509 can be implemented in any number of different ways, with any number of different blocks, sub-blocks, and/or layers that enable the generation of the interaction output 566, the immunogenicity output 568, or both.

In some instances, the machine-learning model 532 may facilitate automated determination as to which particular IPC allele is predicted to bind to a peptide. For example, if an MHC molecule includes twelve MHC allotypes (as is the case for humans), twelve iterations of at least part of a neural-network processing can be performed (e.g., in parallel), one for each allele. Each processing may use, as input, an MHC sequence representation and a peptide representation of at least a portion of the peptide's sequence. Each processing may generate a composite representation. A predicted amino acid-IPC interaction can be determined based on the composite representations. In some embodiments, the predicted amino acid-IPC interaction may comprise a prediction as to whether or an extent to which the peptide will bind to the MHC allele or allotype. It can be inferred that the peptide associated with the highest prediction value (e.g., indicating the most likely binding prediction) across the alleles is the one to which the peptide would bind to.

In some instances, for six up to twelve MHC alleles (e.g., MHC Class I) or allotypes (e.g., MHC Class II) corresponding MHC sequence representations can be generated by running the different MHC allotype sequences through the same BOS+MHC representation block 504 and generating an IPC sequence representation (such as an MHC sequence representation) for each peptide-MHC combination. In some embodiments, the MHC sequence representations can be aggregated together, along with a single appended BOS token that has been embedded with the embedding layer, by multiplying the BOS token of each of the set of transformed amino acid sequence representations (e.g., comprising a set of transformed peptide representations) with the BOS token of the set of transformed IPC sequence representations (e.g., comprising each of the six to twelve MHC sequence representations) to generate composite representations.

In some embodiments, one or more of the processing blocks or sub-blocks included in the machine-learning model 532 can be replaced with another type of network and/or processing unit to convert a representation of one or more sequences. The conversion may represent an extent to which various amino acids (at particular positions) are predicted to influence a binding affinity and/or presentation probability and/or an extent to which various particular combinations of amino acids (at particular positions), occurring over a single sequence or across sequences, are predicted to influence a binding affinity and/or presentation. For example, one or more processing sub-blocks can be replaced by one or more gated recurrent units.

FIG. 5B is a schematic diagram of an example configuration for the machine-learning model 532, in accordance with some embodiments. With the configuration depicted in FIG. 5B, the representation subsystem 501 includes an amino acid sequence representation block 580. The amino acid sequence representation block 580 receives an amino acid sequence. For example, the amino acid sequence may comprise one or more of: a peptide sequence (e.g., peptide sequence 126 in FIG. 1), an N-flank sequence (e.g., N-flank sequence 128 in FIG. 1), or a C-flank sequence (e.g., C-flank sequence 130 in FIG. 1).

Amino acid sequence representation block 580 may include, for example, an embedding layer 582 that processes the amino acid sequence appended with a BOS token to form an embedded amino acid sequence representation received by a positional encoder 583. The positional encoder 583 positionally encodes the embedded amino acid sequence representation to generate BOS+amino acid sequence representation 584. In some embodiments, the BOS+amino acid sequence representation 584 may comprise one or more of: a BOS token representation 581, a peptide representation 585, an N-flank representation 586, or a C-flank representation 587.

The BOS+amino acid sequence representation 584 is output from the amino acid sequence representation block 580 and sent to a processing block 588 in the processing subsystem 503. The processing block 588 includes a set of processing sub-blocks 589 that process the BOS+amino acid sequence representation 584 to generate a transformed BOS+amino acid sequence representation that is sent to the composite block 552 for processing.

In some embodiments, if the amino acid sequence sent to the amino acid sequence representation block 580 includes either an N-flank sequence or a C-flank sequence, but not both, then the machine-learning model 532 may also include the corresponding representation block (e.g., N-flank representation block 506 or C-flank representation block 508 of FIG. 5A) and the corresponding processing block (e.g., processing block 536 or processing block 538, respectively, of FIG. 5A) for the sequence included in the amino acid sequence.

FIG. 5C is a schematic diagram of an example configuration for machine-learning model 532 in accordance with some embodiments. The representation subsystem 501 can be designed to handle one or more subsequences (or combinations thereof) of each type (e.g., peptide, C-flank, N-flank, C-flank+N-flank, MHC, TCR) in order to accommodate concatenation of multiple subsequences by type, together with a single appended BOS token, prior to encoding the concatenated set of subsequences. As shown in the example of FIG. 5C, the representation subsystem 501 may also include a single representation block 507 for a combined sequence including a single BOS token, a C-flank subsequence, and an N-flank subsequence. The representation block 507 may include an embedding layer 521 and a positional encoder 523. The BOS+C-flank+N-flank sequence representation generated by representation block 507 can be transformed by a processor block 590a comprising a set of processing sub-blocks 592a.

Alternatively, the peptide representation, the BOS+peptide sequence representation(s) and a BOS+C-flank+N-flank sequence representation generated by the representation subsystem 501 can be aggregated prior to the transformation stages to form an amino acid sequence representation that is sent into a single processing block 590. The processing block 590 includes a set of processing sub-blocks 592 that process the amino acid sequence representation to generate a transformed amino acid sequence representation that is sent to the composite block 552 for processing.

Similarly, the BOS+MHC sequence representation and a BOS+TCR sequence representation generated by the representation subsystem 501 can be aggregated prior to the transformation stages to form an IPC sequence representation that is sent into the processing block 594. The processing block 594 includes a set of processing sub-blocks 596 that process the IPC sequence representation to generate a transformed IPC sequence representation that is sent to the composite block 552 for processing. In some embodiments, the BOS+MHC sequence representation can be handled by one set of blocks (e.g., 594a, 596a), while the BOS+TCR sequence representation can be handled by another set of blocks (e.g., 594b, 596b).

As shown by FIGS. 5A-5C, the machine-learning model 532 can be implemented in any number of ways using any number of or combination of blocks, sub-blocks, and/or layers within the various subsystems. Thus, the machine-learning model 532 is modular and can be customizable for a given task.

### Example Processing Block

FIG. 6 is a schematic diagram of processing block 600 for executing one or more transformer stages to generate transformed sequence representations, in accordance with some embodiments. Processing block 600 can be one example of an implementation for a processing block in processing subsystem 304 in FIG. 3, or processing subsystem 503 in FIGS. 5A-5C.

Processing block 600 includes one or more processing sub-blocks. For example, the processing block 600 may include processing sub-block 1 602 and, optionally, one or more other processing sub-blocks up to processing sub-block n 604. When a plurality of processing sub-blocks are present in the processing block 600, these processing sub-blocks can be connected serially (e.g., daisy-chained together to produce a final output).

Processing sub-block 1 602 can be implemented in various ways. In some embodiments, processing sub-block 1 602 includes, for example, processing layer 606, add and normalization layer 608, feed forward layer 610, and add and normalization layer 612. With this configuration, the sub-block 1 602 may also be referred as a transformer encoder. In some embodiments, one or more processing sub-blocks 604 can be implemented in a manner similar to the processing sub-block 1 602. In some embodiments, a processing layer 606 may include one or more embedding components configured to perform positional and/or non-positional embedding.

In an add and normalization layer 608 or 612, a transformed representation can be added to the position-indicative embedded representation of a sequence (via a residual connection), and the summed representation can be normalized. The normalized data can be fed to the corresponding feed forward layer 610 (e.g., a fully connected feedforward network). The feedforward network can affect (for example), for each position, one, two, three, or more linear transformations and/or may include an activation (e.g., a ReLU activation) between each of the linear transformations. For example, the feedforward layer can be represented by:

$$FF ( x ) = max ( 0 , {xW}_{1} + {b}_{1} ) {W}_{2} + {b}_{2} ,$$

where x is an input to the layer, W₁ and W₂ are slopes of the linear transformations, and b₁ and b₂ are intercepts of the linear transformation.

The dimensionality of an output of a particular processing sub-block's feed forward layer can be the same as the dimensionality of an input to the processing sub-block's feed forward layer. In some instances, to preserve representations of various types of information, the input and output can be summed and normalized (e.g., via another residual connection through another add and normalization layer).

In some embodiments, the feed forward layer 610 may allow processing of variable length sequences. One or more additional features vectors (e.g., assigned random or pseudorandom values) can be included in a concatenated representation, which is then encoded. This encoded representation of the sequence combination can be processed by a feed forward layer 610 (e.g., a fully connected neural network) where dropout and/or batch normalization can be applied. In some instances, the encoded representation(s) of the additional feature vector(s) are selectively passed to the feed forward layer 610 (e.g., while feature vectors corresponding to individual amino acids of the MHC molecule and/or mutant peptide are not). For example, suppose that a subsequence of an MHC molecule includes x₁ amino acids, a subsequence of a mutant peptide (e.g., and one or more flanks) includes x₂ amino acids, and a feature transformation identifies y feature values to represent each amino acid. A concatenated representation that includes one additional feature vector could thus have a size of [(x₁+x₂+1), y]. The input fed to a feed forward layer 610 may have a size of [1, y], in a case where one feature vector is selected for processing by the feed forward layer 610.

Results produced by the feed forward layer 610 can correspond to predictions as to binding affinities between the mutant peptide and MHC molecule (e.g., an MHC molecule of the subject) and/or whether the mutant peptide will be presented by the MHC molecule. A binding-affinity prediction can be, for example, numeric (e.g., corresponding to a predicted probability that the mutant peptide will bind to the MHC molecule, predicted binding strength, and/or predicted binding stability), categorical (e.g., predicting no, low, or high binding stability between the mutant peptide and the MHC molecule), or binary (e.g., predicting whether the mutant peptide binds to the MHC molecule).

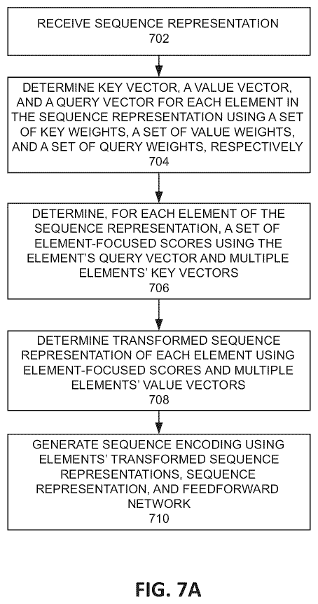

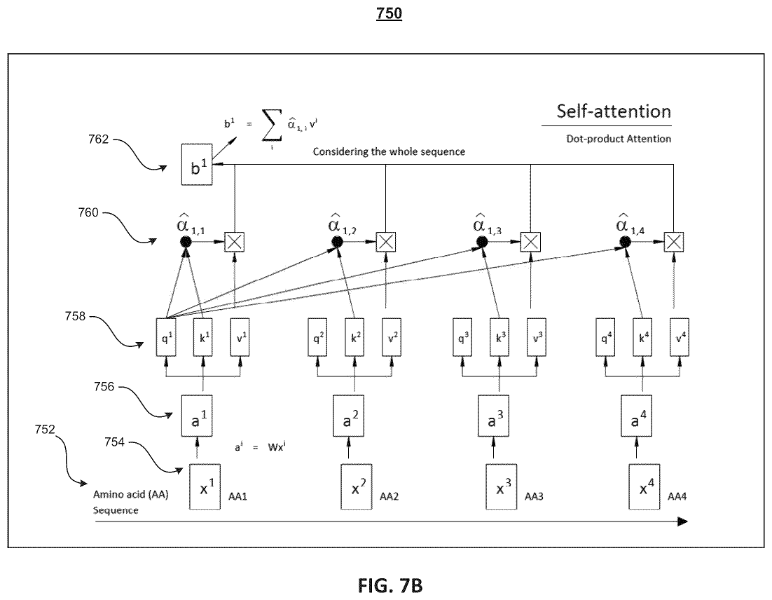

The machine-learning model 132 may include one or more processing layers such as self-attention layers or convolution layers, or a neural network such as a long-short term memory unit (LSTM), recurrent structure, or recurrent component. FIG. 7A illustrates a flowchart of an example process for processing a sequence representation using a processing layer, in accordance with some embodiments. Process 700 can be used by, for example, one or more of the processing blocks present in the machine-learning model 132 in FIGS. 1 and 3, one or more of the processing blocks present in the machine-learning model 532 in FIGS. 5A-5C, and/or the processing block 600 in FIG. 6.

Step 702 includes receiving a sequence representation that includes a plurality of elements. The sequence representation can be, for example, an amino acid sequence representation, an IPC sequence representation, an N-flank sequence representation, a C-flank sequence representation, an MHC sequence representation, a TCR sequence representation, an aggregate sequence representation, or another type of representation. For example, the sequence representation may represent part or all of: a variant-coding sequence, part or all of a sequence that encodes a wild-type or mutant peptide, an epitope sequence (e.g., that includes a variant), a candidate neoepitope sequence, part or all of a neoantigen sequence, a sequence that begins or ends at a terminus of a peptide (e.g., an N-flank or C-flank), or an MHC sequence (e.g., an MHC pseudosequence). The sequence representation can be, for example, generated using representation subsystem 302 in FIG. 3, or representation subsystem 501 in FIGS. 5A-5C. Each element in a sequence representation can be associated with a unique position in the sequence.

Step 704 includes determining a plurality of vectors such as a key vector, a value vector, and a query vector for each element in the sequence representation using a plurality of weights such as a set of key weights, a set of value weights, and a set of query weights, respectively. If, for example, a sequence representation includes, e.g., 20 amino acids, then 20 key vectors, 20 value vectors, and 20 query vectors can be generated. An element in the sequence representation may correspond to, for example, a row or column in a 2-dimensional sequence representation (e.g., where a first dimension represents different amino acids in a sequence and a second dimension represents, for example, different components characterizing individual amino acids).

In some embodiments, the set of key weights are in the form of a key weight matrix. The key weight matrix for a particular element may have a size equal to a length of the element by a length of a key vector. For example, the element may have a length of 20 (e.g., each value corresponding to a binary indication as to whether the amino acid in the sequence is the same as a specific 1 of 21 amino acids), and if a length of a key vector is 5 (e.g., representing 5 components or features), the key weight matrix can have a size of [5, 21]. The key weight matrix can be learned during training and, e.g., randomly initialized at the start of training).

The value vector for an element may have the same size as the key vector for the element. The value vector can be determined using a set of value weights, which can be learned during training and included within a value weight matrix. The value weight matrix for a given element can have the size of the key weight matrix and/or a size based on a length of that element and a length of a value vector.

The query vector for an element may have the same size as the key vector and/or the value vector for the element. The query vector can be determined using a set of query weights, which can be learned during training and included within a query weight matrix. The query weight matrix for an element can have the size of the key weight matrix and/or the value weight matrix. In some embodiments, the query weight matrix may have a size based on the length of the element and the length of a query vector.

Step 706 includes generating, for each element in the sequence representation, a set of element-focused scores using the element's query vector (generated using the query weights and the sequence representation) and multiple elements' key vectors (generated using key weights and the sequence representation). For a given element, the set of element-focused scores can indicate how much weight to give the value vector of the given element. The elements for which the key vectors are used in generating the set of element-focused scores for a selected element in the sequence representation may include some or all of the elements in the sequence representation (e.g., some or all amino acid sequence representations). The elements can include the element of focus (e.g., a particular amino acid for which the set of element-focused scores is being determined).

The set of element-focused scores is generated by generating, for each element of the sequence representation, a score for each pair of the element of focus (the first element) with the same or different element (the second element). The score for this pair can be the product of the first element's query vector and the second element's key vector.

In some instances, step 706 may include implementing an activation function and/or normalization. The normalization can be based on the dimensionality of the key vector (or of the query vector). For example, the normalization can be the square root of the length of a key vector. The activation function can include a softmax function. In some instances, the normalization is applied before the activation function.

Step 708 includes generating a transformed sequence representation. A transformed sequence representation can be determined by performing a transformation of the plurality of elements to form a plurality of modified elements. The transformation can be performed using the set of element-focused scores generated for each of the plurality of elements and the value vector determined for each of the plurality of elements. For example, if a sequence representation includes 11 elements (e.g., representing 11 amino acids), and if scores are determined for all pairwise combinations of the elements, a modified sequence representation comprising a plurality of modified elements is generated. In some embodiments, a modified element can be the weighted average of all elements' value vectors (using the scores for the weighting).

Step 710 includes generating an encoding of the sequence using the transformed sequence representation, the initial sequence representation, and a feedforward network. For example, the transformed sequence representation and initial sequence representation can be summed. This result may still include multiple elements (e.g., each updated via the transformation, summing, and normalization). The feedforward neural network can then process the summed representations (e.g., by performing one, two, or more linear transformations and/or implementing one or more activation functions). Summing the representations can reintroduce positional information that can be obscured in the transformed sequence representation (due to attending to other elements' values when generating a transformed value vector for a given element).

The feedforward neural network can be configured to separately process each of the updated multiple elements (e.g., using a same technique and/or same set of parameters). Thus, the input to the feedforward network can include a vector that corresponds to a single element, single amino acid, and/or single sequence position. The feedforward network can be configured such that an output of the feedforward network is the same size as an input to the feedforward network. In some instances, instead of processing the transformed sequence representation and initial sequence representation using a feedforward network, a convolution (e.g., a 1-dimensional convolution) is instead employed to perform a localized transformation that operates similarly (e.g., identically) across the positions/elements. A 1-dimensional convolutional can be used as another way to interpret the functioning of the feedforward neural network.

The technique illustrated in FIG. 7A pertains to processing using a single set of key vectors, value vectors, and query vectors to calculate the element-focused scores. Embodiments of the disclosure may comprise using a plurality of sets of key weights, value weights, and query weights to produce a distinct key vector, distinct value vector, and distinct query vector. These distinct vectors can be used to produce processing scores and transformed values for each element. Transformed values can be concatenated and projected.

It should be further be appreciated that, while FIG. 7A refers to calculation and use of various vectors, matrix representations may instead be used. Matrix representations may facilitate performing calculations across elements efficiently as opposed to iteratively calculating various vectors individually.

FIG. 7B is a schematic diagram illustrating process 700 described in FIG. 7A in accordance with some embodiments. In FIG. 7B, process 750 receives a sequence 752 as input. The sequence 752 can be, for example, an amino acid sequence. Another example sequence can be an IPC sequence

In the illustrative example in FIG. 7B, the sequence 752 includes a plurality of amino acids 754 (4 amino acids: x¹-x⁴). A sequence representation 756 comprising a plurality of elements a¹-a⁴ is generated via embedding and, in some embodiments, positional encoding. Each element aⁱ can be, for example, a numeric vector. The sequence representation 756 can be one example of the sequence representation received in step 702 in FIG. 7A.

A plurality of vectors 758 (e.g., a query vector qⁱ, key vector kⁱ and value vector vⁱ) can be generated for each element aⁱ in the sequence representation 752. The plurality of vectors 758 can be examples of implementations for the vectors generated in step 704 in FIG. 7A. The illustrated example corresponds to generating select element-focused scores 760, â_{1,i}, with a focus on the first element, a¹. The element-focused scores 760 are an example of one set of element-focused scores generated for a particular element in step 706 in FIG. 7A. Each of the element-focused scores â_{1,i} can be the dot product of q¹ with kⁱ. The weighted sum of the value vectors vⁱ, with the weights being set to â_{1,i}, are computed to perform a transformation that generated a modified element 762, b¹. The modified element 762 is one example of a modified element generated in step 708 in FIG. 7A. Similar transformations can be performed for the other elements of the sequence representation 756. Additional details for example transformer architectures can be found in Ashish Vaswani et al., Attention is All You Need, Neural Information Processing Systems (2017).

### Example Methods for Using a Machine-Learning Model

Machine-learning model 132 in FIGS. 1 and 3, workflows in FIGS. 4A-D, and machine-learning model 532 in FIGS. 5A-5C can be used in various ways to generate predictions about the immunological activity (e.g., predicted binding, binding affinity, predicted presentation occurrence, immunogenicity, etc.) associated with various peptides, including mutant peptides (e.g., neoantigens).

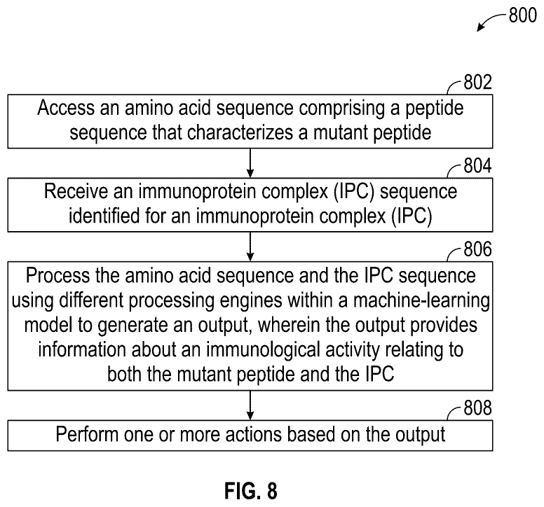

FIG. 8 is a flowchart of an example process for generating information about the immunological activity of various peptides, in accordance with some embodiments. At least a portion of process 800 can be implemented using, for example, without limitation, prediction system 100 described in FIG. 1. For example, at least a portion of process 800 can be implemented using, for example, without limitation, machine-learning model 132 from FIGS. 1 and 3, machine-learning model 532 from FIGS. 5A-5C, or the workflows in FIGS. 4A-D.

Step 802 includes accessing an amino acid sequence comprising a peptide sequence that characterizes a mutant peptide, the peptide sequence may include a variant with respect to a corresponding reference sequence. The peptide sequence characterizes the mutant peptide by characterizing at least a portion of the mutant peptide. The mutant peptide can be, for example, a neoantigen. Step 802 can be performed by, for example, retrieving the peptide sequence from a data store (e.g., data store 104 in FIG. 1, a cloud storage, a server or server system, etc.). In some embodiments, the peptide sequence can be one of a plurality of peptide sequences that are processed through a machine-learning model.

Step 804 includes receiving an IPC sequence identified for an IPC of a subject. The IPC can be, for example, an MHC, a TCR, or an MHC-TCR complex. The IPC sequence characterizes the IPC by characterizing at least a portion of the IPC.

Step 806 includes processing the amino acid sequence and the IPC sequence using different processing engines within a machine-learning model to generate an output, wherein the output provides information about an immunological activity relating to both the mutant peptide and the IPC. Step 806 includes, for example, processing the amino acid sequence through a corresponding representation block to generate an amino acid sequence representation. The amino acid sequence representation can be processed through a corresponding processing block to generate a transformed amino acid sequence representation. This amino acid processing engine is separate and independent from the IPC processing engine in which the IPC sequence is processed through a corresponding representation block to generate an IPC sequence representation (e.g., an MHC representation, a TCR representation, an MHC-TCR representation) that is processed through a corresponding processing block to generate a transformed IPC sequence representation (e.g., a transformed MHC representation, a transformed TCR representation, a transformed MHC-TCR representation) that represents the IPC sequence.

In some embodiments, the amino acid sequence representation is an aggregate representation that includes an N-flank representation for an N-flank sequence and/or a C-flank representation for a C-flank sequence. In such embodiments, the aggregate processing engine (which may include the amino acid processing engine) remains separate from the IPC processing engine.

In various embodiments, in step 806, the transformed amino acid sequence representation and the transformed IPC sequence representation are used to form a composite representation that is then further processed to generate the output. The output may include, for example, without limitation, a set of interaction predictions, a set of interaction affinity predictions, a set of immunogenicity predictions, or a combination thereof.

Step 808 includes performing one or more actions based on the output. As one example, a report including the output can be generated. In some embodiments, the report includes a transformed or filtered version of the output. In some embodiments, the report includes a summary, synopsis, or a visual representation of the output.

In some embodiments, step 808 comprises other actions relating to the design and/or manufacturing of a treatment based on the output. For example, a pharmaceutical composition can be selected or ranked based on the output. The output may comprise a prediction of which mutant peptides bind to a subject's specific IPC (e.g., MHC allele or allotype). This binding prediction may indicate the likelihood that the subject's immune system may recognize, e.g., cancerous cells. The binding prediction can be used to help select candidate neoepitopes (mutant peptides) for a vaccine. In some embodiments, the composite representation having the highest result(s) (e.g., prediction value indicating the most likely binding and/or presentation prediction) in the output can be selected for a pharmaceutical composition. In some embodiments, the composition representations can be ranked according to corresponding results in the output.

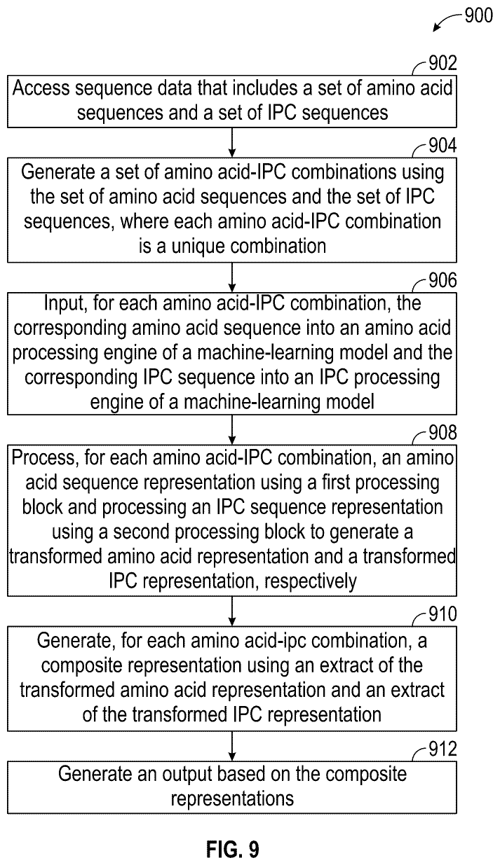

Embodiments of the disclosure may include generating an output based on a set of IPC sequences. For example, for a given subject, the output can be generated based on six up to twelve MHC alleles or allotypes. FIG. 9 is a flowchart of an example process for generating information about the immunological activity of various peptides, in accordance with some embodiments. At least a portion of process 900 can be implemented using, for example, without limitation, prediction system 100 described in FIG. 1. For example, at least a portion of process 900 can be implemented using, for example, without limitation, machine-learning model 132 from FIGS. 1 and 3, or machine-learning model 532 from FIGS. 5A-5C.

Step 902 includes accessing sequence data that includes a set of amino acid sequences and a set of IPC sequences.

Step 904 includes generating a set of amino acid-IPC combinations using the set of amino acid sequences and the set of IPC sequences. Each amino acid-IPC combination is a unique combination.

Step 906 includes inputting, for each amino acid-IPC combination, the corresponding amino acid sequence into an amino acid processing engine of a machine-learning model and the corresponding IPC sequence into an IPC processing engine of a machine-learning model.

Step 908 includes processing, for each amino acid-IPC combination, an amino acid sequence representation using a first processing block and processing an IPC sequence representation using a second processing block to generate a transformed amino acid sequence representation and a transformed IPC sequence representation, respectively.

Step 910 includes generating, for each amino acid-IPC combination, a composite representation using the transformed amino acid sequence representation and the transformed IPC sequence representation.

Step 912 includes generating an output based on the composite representations. In some embodiments, the predicted amino acid-IPC interaction can be determined based on the composite representations. The output may provide an indication of which of the peptide sequences can be used to generate a treatment. For example, the output may provide an indication of which peptide sequences (and thereby, a peptide that contains that peptide sequence) has a high likelihood of binding to an MHC, a high likelihood of being presented by an MHC, a high interaction affinity for the peptide-MHC binding, and/or a high likelihood of being immunogenic to thereby trigger an immune response.

### Example Methods for Training a Machine-Learning Model

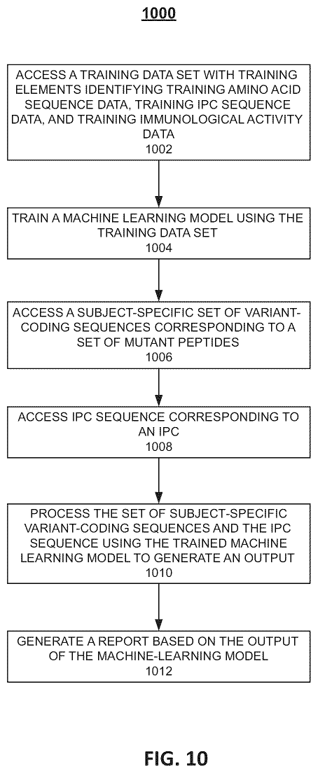

FIG. 10 is a flowchart of an example process for training a machine-learning model and using the trained machine-learning model to generate predictions relating to amino acids (e.g., peptides) and IPCs (e.g., MHCs), in accordance with some embodiments. Process 1000 can be performed using the prediction system 100 in FIG. 1. For example, process 1000 can be implemented using machine-learning model 132 in FIGS. 1 and 3, machine-learning model 532 in FIGS. 5A-5C, or any workflows in FIGS. 4A-D. In some instances, part or all of process 1000 can be performed at a remote computing system that is remote relative to a user device and/or laboratory. The remote computing system can be a cloud computing system.

A machine-learning model can be trained using at least part of the training data set. Step 1002 includes accessing a training data set with training elements identifying training amino acid sequence data, training IPC sequence data, and training immunological activity data. The training data set can be one example of an implementation for training data 133 in FIG. 1. The training immunological activity data may include, for example, interaction indications.

The training data set can include multiple training data elements. Each training data element can include a sequence representation and a result (e.g., indicating whether at least part of a peptide corresponding to the sequence is presented by an MHC molecule and/or triggers immunogenicity). Training data elements for which presentation or binding was not detected can be generated computationally. For example, for each protein of origin in the positive set (corresponding to positive eluted-ligand presentation data), one or more (e.g., all) possible peptide fragments (e.g., within a predetermined length range, such as from 8 to 11) can be generated, potentially with uniform probability, for each length. N-terminal and C-terminal flanking sequences can be retained (e.g., potentially with a maximum length, such as 10 amino acids). In some instances, for each allele represented in positive instances in the training data, peptide fragments (e.g., of one or more (e.g., all) lengths of 8:11) can be generated. The generation and/or subsequent selection can be performed such that a probability of occurrence of a sequence having a given length is uniform across lengths. N-terminal and C-terminal flanking sequences can be or may have been retained with a particular maximum length (e.g., a maximum length of 10 amino acids). In particular embodiments, any other suitable sequence length range (e.g., 9-30 for MHC Class II) can be utilized.

The training data set can be randomly parsed, shuffled, and/or divided to train various models within the ensemble. A loss function can use an error term (e.g., mean squared error or median squared error) and/or an entropy term (e.g., cross entropy or binary cross entropy). Multitask learning can be used, such that the model is simultaneously trained to predict each of two different types of results (e.g., binding affinity and presentation occurrence). A static or non-static learning rate can be used. For example, learning rate annealing (e.g., using stepwise annealing or cosine annealing) can be used to reduce the learning rate over iterations. Validation-data assessment can be used to potentially terminate training early (e.g., upon determining that a performance target has been met).

The training amino acid sequence data may include, for example, one or more amino acid sequences (which may include variant-coding sequences) for training. An amino acid sequence may comprise a peptide sequence. A peptide sequence can identify an ordered set of amino acids within a peptide (e.g., a neoantigen). The peptide sequence can identify amino acids within an epitope (e.g., includes a variant, includes a neoepitope, and/or is a neoepitope) of the peptide. In some embodiments, the peptide sequence is within an aggregate sequence that also includes an N-flank sequence (e.g., characterizing a chain of amino acids at an N-terminus of the corresponding peptide) or a C-flank sequence (e.g., characterizing a chain of amino acids at a C-terminus of the corresponding peptide). Neither the N-flank nor the C-flank bind to an MHC molecule, though each may influence whether it is presented by an MHC molecule.

In some instances, it is not known how many amino acids from a flank (e.g., N-flank) are used by peptidases to determine when to trim long peptides into a peptide core that is presented. To address this unknown in generating the training data, flanks may then be trimmed to a length selected based on a technique (e.g., pseudo-random selection technique), such as a length within a predetermined range (e.g., 1-10 amino acids). The selection technique may select a length using a distribution (e.g., uniform or Gaussian distribution). In some instances, a flank that is below a threshold length (e.g., 10 amino acids) is not trimmed. In some instances, a flank trimming can be such that the C side on an N-flank is preserved.

The training MHC sequence data may include one or more MHC sequences for training. An MHC sequence may, for example, identify amino acids within part or all of an MHC molecule (e.g., an MHC-I molecule or an MHC-II molecule). The MHC sequence can include an MHC pseudosequence (e.g., that includes 34 amino acids). The MHC sequence can identify amino acids within, for example, 1, 2, 3, 4, 5 or 6 MHC alleles for MHC-I, or 1, 2, 3, 4, 5, 6, 7, 8, 9, 10, 11, or 12 MHC allotypes for MHC-II. The MHC sequence can identify amino acids constituting part or all of an HLA molecule.

The MHC includes multiple alleles in vivo (e.g., six alleles and twelve allotypes per human). For a single MHC molecule, multiple sequence inputs can be generated (e.g., each representing a single allele of the multiple alleles). Each of the multiple sequence inputs can be separately processed using the one or more neural networks (e.g., one or more transformer encoders) so as to generate a predicted binding or presentation value of a neoantigen in association with each of the alleles. A function (e.g., max function) can identify which allele from among the multiple alleles is associated with the highest presentation prediction. During training, this maximum presentation prediction for this particular sequence input can then be compared to a true presentation value using a binary loss function to generate errors for tuning parameters.

The training immunological activity data may include, for example, one or more interaction indications for one or more amino acid-IPC combinations. For example, the training data set may include training elements, in which each training element includes an amino acid sequence and an IPC sequence for training, as well as one or more interaction indications for the corresponding amino acid-IPC combination. An interaction indication may indicate whether a target interaction (e.g., binding of a peptide and MHC, presentation of a peptide on the cell surface by MHC) occurs between an amino acid (e.g., peptide) and IPC (e.g., MHC) or an affinity for the target interaction and/or triggers an immunological response.

The interaction indication can be, for example, a label. A negative interaction label may indicate that a peptide does not bind to and/or is not presented by an IPC (e.g., an MHC molecule). A positive interaction label may indicate that a peptide binds to and/or is presented by an MHC molecule. Further, an interaction label may indicate the probability that the peptide binds to the MHC molecule, a binding affinity for the peptide-MHC combination, the strength of the binding between the peptide and the MHC molecule, the stability of the binding between the peptide and the MHC molecule, the tendency of the peptide to bind with the MHC, or another metric or characteristic associated with an interaction between the MHC and the peptide.

The training data set may have been generated via, for example, in vitro or in vivo experiments and/or based on medical records. In some embodiments, the machine-learning model can be trained using binding-affinity data and mass-spectrometry elution data indicating which peptides are presented by MHC molecules. The binding-affinity data may include qualitative data (e.g., as determined using ELISAs, pull-down assays and/or gel-shift assays, fluorescence resonance energy transfer assays, and mass spectrometry assays) or quantitative data (e.g., using a biosensor-based methodology, such as Surface Plasmon Resonance, Isothermal Titration Colorimetry, BioLayer Interferometry, or MicroScale Thermophoresis). In some instances, binding affinity data can include data from a competitive binding assay, data from the Immune Epitope Database, and/or data of a type that is in the Immune Epitope Database. Elution data can be collected using peptide-MHC immunoprecipitation, followed by elution and detection of presented MHC ligands by mass spectrometry.

To collect training data, some of the sequences identified in a disease sample can be non-disease sequences that correspond to non-disease peptides. To identify disease-specific nucleic acid sequences and/or disease-specific amino acid sequences, for each sequence that is detected as a result of sequencing the disease-specific sample, it can be determined whether the sequence is also identified in a reference sequence data set. The reference sequence data set can include a set of reference sequences for which it is known, inferred, or assumed that the sequence is not indicative or characteristic of a disease (e.g., any disease or a given disease). The reference sequence data set may, for example, include sequences identified by sequencing one or more reference sample sequences collected from the same subject from which the disease-specific sample was collected, sequencing one or more reference sample sequences collected from one or more other subjects not diagnosed with any disease or a disease corresponding to the disease-specific sample, and/or sequencing one or more cell lines not associated with the specific disease. In some instances, the reference sequence data set may include sequences collected from one or more reference data repositories. A sequence that is detected in association with the disease-specific sample but that is not detected (or detected at a frequency below a pre-determined threshold) in a reference sequence data set can be classified as a variant-coding sequence (e.g., generally or for a subject from which the disease-specific sample was collected).

In some instances, multiple variant-coding sequences can be identified (e.g., each having been detected in the disease sample, but not represented in the reference-sample sequences). In some instances, a representation of each of the multiple variant-coding sequences can be processed (e.g., individually, sequentially, and/or in parallel) using a machine-learning model disclosed herein to predict the binding affinity and/or presentation prediction.

The disease sample can include, for example, tissue (e.g., a solid tumor), blood, and/or a collection of cells (e.g., cancer cells, which may have been collected using fine needle aspiration or laparoscopy). The disease sample may include cancerous cells collected from a subject that has been diagnosed with and/or that has, for example, lung cancer, melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, and T cell lymphocytic leukemia, non-small cell lung cancer, or small cell lung cancer.

In some instances, an initial sample is separated into a disease sample and another remainder sample (e.g., which can be discarded or used as a reference sample). The reference sample can include a matched disease-free sample. Each of the disease sample and the reference sample can be collected from the same subject and/or may include or be of the same or similar sample type (e.g., tissue type). In some instances, the disease sample is collected from a first subject (e.g., who has been diagnosed with a medical condition or disease), and the reference sample is collected from a different, second subject (e.g., who has not been diagnosed with the medical condition or disease). In some instances, the reference-sample sequences are retrieved from a database of known genes associated with an organism.

Training data may further include sequences of one or more peptides, along with indications as to whether each of the peptides is bound to an MHC molecule, presented by an MHC molecule, and/or triggered an immunological response. To collect training data that associates sequence data with observed presentation and/or binding data, the disease sample (and potentially the reference sample) can be (separately) processed to isolate MHC/peptide complexes (e.g., by performing immunoprecipitation using an antibody specific for MHC) and/or eluting (and thereby sequencing) the peptides from the MHC molecules (e.g., using chromatography and/or mass spectrometry). In some instances, reference-sample sequences are identified for use in generating presentation data by sequencing one or more cell lines engineered to express one or more MHC alleles (e.g., that were detected in the disease sample), which can include MHC class-I alleles and/or MHC class-II allotypes. The one or more cell lines can include one or more human cell lines obtained or derived from one or more subjects. For purposes of this description, peptide sequences that are identified using a disease sample but that are not represented in a set of reference-sample sequences can be identified as variant-coding sequences.

In some embodiments, collecting immunogenicity-indicative metrics to use for training can be based on HLA-typing analysis, which can identify a subject-specific MHC molecule profile. When the subject is a human, this profile can be referred to as a Human Leukocyte Antigen (HLA) profile, as the HLA complex is a gene complex encoding MHC proteins in humans. An HLA-typing analysis can be performed using a sample (e.g., normal-tissue and/or non-disease sample) from the subject. The profile can be determined using a sequencing technique, such as PCR-based sequencing, direct sequencing, and/or next-generation sequencing. The HLA-typing analysis may include, for example, high-resolution typing (e.g., which excludes indicating null alleles that are not expressed on the cell surface) or allele-level typing (e.g., which refers to exact nucleotide sequence HLA-gene determination). The HLA-typing analysis may include low-resolution typing and/or HLA supertyping that identifies broader families of alleles.

With respect to any type of sequencing (e.g., to identify sequences in a sample, peptides bond to an MHC molecule, HLA typing), a result may identify one or more nucleic acid sequences or one or more amino acid sequences. When nucleic acid sequences are identified and an attention-based model (or other processing) is configured to process amino acid sequences, a technique (e.g., lookup table) can be used to convert individual codons within the nucleic acid sequences into individual amino acids.

Some embodiments including synthesizing a peptide (e.g., using a nucleic acid sequence encoding a peptide, such as a selected peptide) or a precursor to a selected peptide. The synthesized peptide or precursor may then be used in an experiment to identify corresponding presentation and/or binding data (e.g., to verify predicted presentation and/or binding or to generate results to use for training). For example, an experiment may include assessing binding affinity of a selected peptide with a particular MHC molecule using an ELISA pull-down assay, gel-shift assays, or a biosensor-based methodology. As another example, an experiment may include collecting elution data indicative of whether a selected peptide was presented by an MHC molecule by using peptide-MHC immunoprecipitation, followed by elution and detection of presented MHC ligands by mass spectrometry.

In addition to or instead of training or verification data indicating whether individual peptides bound to and/or were presented by individual MHCs, training or verification data may indicate whether individual peptides triggered immunogenicity. Immunogenicity results can be determined using in vivo or in vitro testing. Testing the one or more selected peptides can be configured to investigate one or more immunogenicity factors (e.g., to determine whether and/or an extent to which a given event occurs) and/or immunogenicity (e.g., to determine whether and/or an extent to which the peptide triggers an immunological response). Testing can be configured to investigate whether administration of a composition (e.g., a vaccine) that includes one or more peptides to a given subject (e.g., for which an MHC sequence that was used during mutant-peptide selection has been identified) is effective in preventing or treating a medical condition (e.g., tumor) or disease (e.g., cancer). The subject can be a human subject.

Accessing the training data set may include, for example, retrieving the training data set from a local or remote storage, loading the training data set, and/or requesting (and receiving) part or all of the training data set from one or more data stores (e.g., a cloud data storage, a server system, or some other data source).

Training data may include “positive” instances (e.g., for which mass-spectrometry results indicate that a peptide was presented by an MHC molecule) and “negative” instances (corresponding to, for example, simulated length-matched n-mers (nmers)) from the same proteins as positive instances (e.g., but that were not detected in mass-spectrometry assessments).

In some instances, an initial training data set (e.g., which may include variant-coding sequences) may include predominately negative data, in that a relatively small portion of the sequence combinations (e.g., peptide-MHC combinations) is found to be associated with an actual target interaction. The training data set can be designed to include negative training data elements. In some embodiments, a negative training data element can be used to identify amino acids within a pseudo-randomly selected fragment of a protein of origin in the positive set (corresponding to observed presentation). For example, the negative training data element can be simulated based on the positive set. The fragment can be selected to have a length within a predetermined range (e.g., between 8 and 14 amino acids for MHC-I and 8-30 amino acids for MHC-II, using a uniform probability). N-terminal and C-terminal flanking sequences can be retained within the negative training data element, potentially imposing a maximum length (e.g., of 10 amino acids). Any peptide fragment (e.g., at least a 9-mer) that overlapped with a positive peptide can be discarded from the negative training data.

In some embodiments, the negative training data elements are simulated based on the positive data elements. Further, the training data is selected such that a different set of negative training data elements is used per epoch of the training period. For example, for each epoch, a different “negative subset” of negative peptide sequences can be selected from the overall space of available negative peptide sequences identified based on the positive set of peptide sequences. The negative subset selected for each epoch can be unique in that no negative peptide sequence is repeated in any of the negative subsets for the total number of epochs. Thus, the training data used for each epoch of the training period includes the same positive set of peptide sequences but an entirely different set of negative peptide sequences. This technique, which can be referred to as negative set switching may provide overall robustness to the training and helps to ensure either a reduced number of false negatives (e.g., false negative indications/predictions) by the machine-learning model or that no false negative is repeated more than once. Further, with this technique, the machine-learning model can be trained on a total number of negative peptide sequences that is equal to the number of positive peptide sequences multiplied by the number of epochs in the training period.

In some examples, the number of positive instances in the training data is equal to the number of negative instances in the training data. In some examples, the number of positive instances is less than or greater than the number of negative instances. Each of one or more (e.g., all) of the negative instances in the training data can be length-matched to a positive instance in the training data. In some examples, all of the sequences in the training data have the same length.

Step 1004 includes training a machine-learning model using the training data set. The machine-learning model can be, for example, machine-learning model 132 in FIGS. 1 and 3, or the machine-learning model can be, for example, machine-learning model 532 in FIGS. 5A-5C.

The machine-learning model can be trained using a static or dynamic learning rate. A dynamic learning rate can be produced using, for example, learning-rate annealing. Training can be performed using, for example, a classification loss function and/or a regression loss function. A loss function can be based on, for example, mean square error, median square error, mean absolute error, median absolute error, an entropy-based error, a cross entropy error, and/or a binary cross entropy error. Validation data (e.g., a separated subset of the training data set used to train the machine-learning model can be used to assess the performance of the machine-learning model as it is being trained. Training can be terminated if and/or when the target performance is obtained, and/or the maximum number of training iterations have been completed.

Step 1006 includes accessing a subject-specific set of variant-coding sequences corresponding to a set of mutant peptides. As described above, a variant-coding sequence is one example of a peptide sequence. The subject-specific set of variant-coding sequences can correspond to a set of mutant peptides, such that each of the subject-specific set of variant-coding sequences identifies amino acids within a corresponding mutant peptide of the set of mutant peptides. In some embodiments, each of the subject-specific set of variant-coding sequences identifies one or more amino acids in a mutation. Each of the subject-specific set of variant-coding sequences can be associated with a particular subject (e.g., human subject). The particular subject may have been diagnosed, experienced symptoms, and/or received test results associated with a particular medical condition (e.g., cancer). For example, the subject-specific set of variant-coding sequences may have been identified by processing a sample from a tumor. The sample can be included within, for example, the set of samples 112 in FIG. 1.

The subject-specific set of variant-coding sequences can be identified using a technique disclosed herein. For example, the subject-specific set of variant-coding sequencing may have been identified by performing a sequencing technique to identify peptides in a disease sample and comparing the identified peptides to those detected in a healthy sample or reference database to identify unique sequences. In some embodiments, if the unique sequences are nucleic acid sequences, each unique nucleic acid sequence can be transformed into an amino acid sequence.

Each of the subject-specific set of variant-coding sequences can identify amino acids within a peptide (which can be amino acids within the neoepitope of a neoantigen). In some instances, each of one, more, or all the subject-specific set of variant-coding sequences can be part of a corresponding aggregate sequence that further includes a sequence at an N-flank of the peptide and/or a sequence at a C-flank of the peptide.

Accessing the subject-specific set of variant-coding sequences can include, for example, retrieving the subject-specific set of variant-coding sequences from a local or remote storage and/or requesting the subject-specific set of variant-coding sequences from another device. Accessing the subject-specific set of variant-coding sequences can include and/or can be performed in combination with determining the subject-specific set of variant-coding sequences.

The subject-specific set of variant-coding sequences may have been obtained by identifying peptide sequences within a disease sample of the subject and determining which of the peptide sequences are not represented within a reference, healthy-sample, and/or wild-type sequence set. In instances in which a healthy sample is used for the comparison, the healthy sample may have been (but need not have been) collected from the subject.

Step 1008 includes accessing an IPC sequence corresponding to an IPC. In some embodiments, the IPC sequence can be an MHC sequence. The MHC sequence may include, for example, a pseudosequence of an MHC (e.g., MHC molecule) within the sample collected from a subject. In some instances, the MHC sequence and the subject-specific set of variant-coding sequences are identified from the same sample from the subject or from multiple samples from the subject (e.g., a disease sample and a healthy sample). In some instances, the MHC sequence and the subject-specific set of variant-coding sequences are identified from samples from the subject and one or more other subjects. Thus, in some cases, the MHC sequence can be subject-specific. The MHC sequence can be or may have been determined using, for example, a sequencing and/or mass-spectrometry technique.

Accessing the MHC sequence may include, for example, retrieving the MHC sequence from a local or remote storage and/or requesting the subject-specific MHC sequence from another device. Accessing the MHC sequence can include and/or be performed in combination with determining the MHC sequence.

Step 1010 includes, for example, processing the set of subject-specific variant-coding sequences and the MHC sequence using the trained machine-learning model to generate an output. Step 1010 may include processing each unique combination (e.g., variant-coding-MHC combination or peptide-MHC combination) of a subject-specific variant-coding sequence of the set of subject-specific variant-coding sequences and the MHC sequence to generate the output.

The output generated by the machine-learning model can be include the same or similar type of data as included in the training immunological activity data used to train the machine-learning model. For each unique combination, the machine-learning model generates an output that includes at least one of a set of interaction predictions or a set of interaction affinity predictions.

An interaction prediction in the set of interaction predictions includes a prediction about whether a target interaction between a mutant peptide (that includes the variant-coding sequence) and an MHC (that includes the MHC sequence) will occur. For example, the interaction prediction may include a binary or categorical prediction as to whether a mutant peptide with an amino acid structure (as indicated by the subject-specific variant-coding sequence) will be presented by and/or bind to an MHC molecule (with an amino acid structure as indicated by the MHC sequence). An interaction affinity prediction in the set of interaction affinity predictions includes a prediction about an affinity for the target interaction. This affinity can be based on, for example, the strength, tendency, and/or stability of the target interaction. For example, the interaction affinity prediction may include a predicted real-number binding affinity associated with a mutant peptide that includes amino acids identified within the subject-specific variant-coding sequence and an MHC molecule including amino acids as identified within the MHC sequence.

Step 1012 includes generating a report based on the output of the machine-learning model. The report can be implemented as, for example, report 144 in FIGS. 1 and 3. The report can be or include the output. In some cases, the report can be a transformed or filtered version of the output.

In some embodiments, the subject-specific set of variant-coding sequences is filtered, ranked, and/or otherwise processed based on the output to generate information for inclusion in the report. For example, the subject-specific set of variant-coding sequences can be filtered to exclude sequences for which a predicted interaction affinity (e.g., binding affinity) was below a predetermined affinity threshold and/or it was predicted that the target interaction (e.g., binding to the MHC molecule) would not or would be unlikely to occur. In some instances, filtering is performed to identify a predetermined number and/or fraction of the subject-specific set of variant-coding sequences. For example, filtering can be performed to identify 10, 20, 40, 60, 80, 100, 500, or 1,000 variant-coding sequences associated with relatively high predicted probabilities (e.g., relative to unselected variant-coding sequences in the subject-specific set of variant-coding sequences) as to whether the mutant peptide will bind to an MHC molecule.

The report may identify one or more variant-coding sequences (e.g., that were not filtered out from the set) and/or one or more mutant peptides (e.g., associated with selected variant-coding sequences). A mutant peptide can be identified by, for example, its name, its sequence, and/or identifying both a corresponding wild-type sequence and a variant represented in a variant-coding sequence.

The report may identify one or more predictions associated with one or more variant-coding sequences or one or more mutant peptides. The report may include the name of the subject. The report may, for example, be presented locally (e.g., for display on a display system of a user device, sent as a notification on a user device, etc.) and/or transmitted to another device (e.g., sent to a cloud computing system, sent to a cloud storage, sent to a user device associated with a medical profession or laboratory professional, transmitted as an email, etc.).

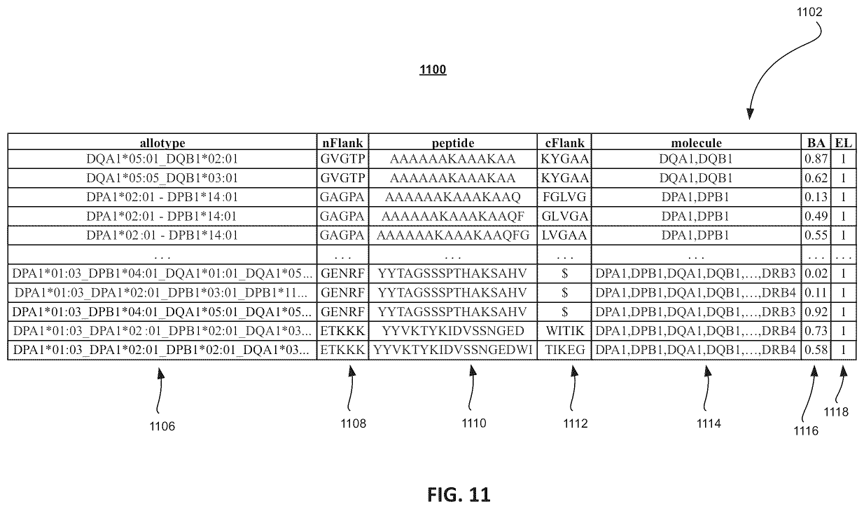

FIG. 11 is an illustration that includes an example table of training data, in accordance with some embodiments. Table 1100 comprises training data 1102 (e.g., a training data set). Training data 1102 can be one example of a portion of training data 133 in FIG. 1. Training data 1102 can be one example of a portion of a training data set such as the training dataset described in step 1002 in FIG. 10.

Training data 1102 includes allotype identifier 1106, training N-flank sequence 1108, training peptide sequence 1110, training C-flank sequence 1112, and training MHC sequence 1114 (e.g., MHC pseudosequence), binding affinity 1116 (e.g., a normalized binding affinity scaled between 0 and 1) and presentation indication (e.g., elution likelihood) 1118. Binding affinity 1116 indicates the detected (e.g., observed) binding affinity for the binding of the peptide characterized by training peptide sequence 1110 and the respective MHC characterized by training MHC sequence 1114. Presentation indication 1118 indicates whether the binding or presentation of the peptide by the MHC was detected (or observed).

### Example Predictions

Embodiments of the disclosure may comprise determining one or more predictions including, but not limited to, immunogenicity, binding affinity, and potential interactions between a mutant peptide and an MHC molecule.

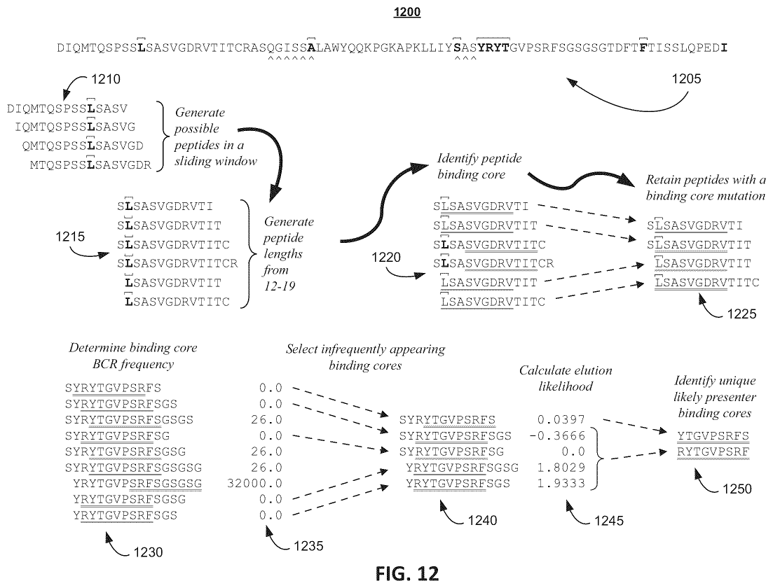

FIG. 12 is an example method 1200 for predicting which therapeutic antibodies are likely to increase immunogenicity risk. As shown in FIG. 12, an example sequence 1205 for a light chain of a therapeutic antibody may include various amino acid mutations relative to a germline (as denoted by bold letters with an overhead square bracket), as well as various complementarity-determining regions (CDRs), as denoted by carats underneath the letter(s). A set of all possible peptides 1210 can be generated using a sliding window (e.g., for a given peptide sequence length, within a range of 9-30 amino acids), and candidate peptides 1215 can be identified within specified lengths (e.g., 12-19 amino acids). For each of the candidate peptides 1215, a binding core can be identified (as shown in FIG. 12 using a double underline—see 1220). The set of candidate peptides 1215 may then be filtered to retain only those peptides whose binding core includes a mutation (see 1225). By filtering out peptides where the binding core does not include any mutation, the method eliminates those peptides that will not be immunogenic due to its similarity to a human peptide.

Next, for the set of candidate peptides that have a binding core that includes a mutation 1230, the method determines a frequency 1235 with which the binding core appears in a database of B-cell receptor binding cores appearing in healthy people (e.g., a frequency of 9-mers obtained from B-cell receptors). If the frequency is high, then the candidate peptide is filtered out (again, to eliminate peptides that are unlikely to be immunogenic), so that only those candidate peptides having binding cores that do not appear or only infrequently appear in the database remain (see 1240). Next, a presentation likelihood (e.g., elution likelihood) 1245 is calculated for those remaining candidate peptides. The presentation likelihood can be calculated using the methods and systems discussed in the remainder of this description, for example as discussed with respect to FIGS. 1-11 as above. After filtering out any candidate peptides having a negative presentation likelihood, the method identifies the set of unique binding cores from the remaining candidate peptides, thereby arriving at the set of unique likely presenter binding cores 1250. In some embodiments, the method may go on to count a number of unique binding cores for each allele and compute a sum over all MHC I alleles and/or MHCII allotypes. The results of this calculation may inform a decision about whether the therapeutic antibody represents a risk of immunogenicity in subjects. This risk may take the form of a count of unique, likely presenting binding cores, or some other score such as the number of uniquely presenting binding cores weighted by the elution likelihood, optionally in combination with other categorical or numerical information.

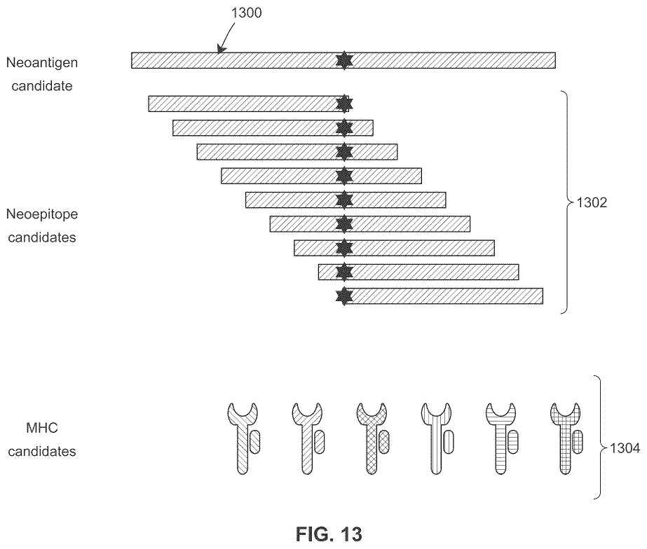

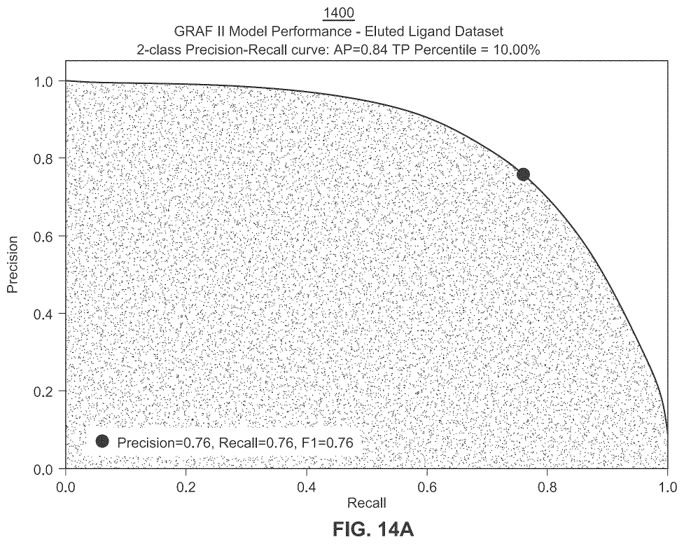

FIG. 13 is an illustration of an example neoantigen candidate (mutant antigen) and the corresponding potential neoepitope candidates (mutant peptides), in accordance with some embodiments. When a process such as process 1000 is implemented, a mutant peptide can be a neoantigen.

For a relatively long mutant peptide that is a neoantigen candidate 1300, it is possible that multiple epitopes (referred to as neoepitopes), all containing the same mutation or variant, can be presented by an MHC molecule. Thus, the immunogenicity of the neoantigen candidate can be predicted based on predictions generated for each of the neoepitope candidates 1302.

The immunogenicity can be predicted by, for example, generating a list of all possible neoepitopes that could emerge from a given neoantigen and producing predictions for each of some or all of the neoepitope candidates (with the flanks constituting the remaining amino acids upstream of the N-terminus and downstream of C-terminus of the epitope, up to 10 amino acids in length) in the list. From these presentation predictions, the neoepitope candidate with the largest presentation likelihood with respect to the MHC candidates 1304 is chosen to represent the entire neoantigen. Alternatively, a summarized representation of multiple candidate neoepitope-MHC pairs can be used to obtain a summarized score representing the neoantigen. Such summarization can be conducted by either considering all candidate neoepitope-MHC pairs or by considering the best neoepitope per MHC and then summarizing across all MHC molecules. The summarization can be done by several mathematical functions including, for example, taking the arithmetic mean or harmonic mean of the presentation or binding affinity score of each candidate neoepitope-HLA pair.

Although FIG. 13 is described with respect to neoantigens and neoepitopes, a similar technique can be used for other types of relatively long mutant peptides containing a mutation or variant and having multiple possible epitope candidates. In some embodiments, this technique can be used in conjunction with antibody drug sequences.

In some embodiments, it can be predicted that a neoantigen detected from a subject's disease sample will not trigger immunogenicity or will have low immunogenicity when a machine-learning-model result predicts that the mutant peptide will have low binding affinity with an MHC molecule. In some embodiments, it can be predicted that an MHC molecule will not or is not likely to present the mutant peptide. In some embodiments, it can be predicted that a mutant peptide will not trigger an immunological response by a T-cell receptor. An immunogenicity prediction generated in association with a mutant peptide can be, for example, numeric (e.g., corresponding to a predicted probability that an immunogenicity response would be triggered in response to the mutant peptide and/or corresponding to a predicted intensity of any immunogenicity response to the mutant peptide), categorical (e.g., predicting no, low, or high immunological response) or binary (e.g., predicting whether a given mutant peptide triggers an immunological response in the subject).

A predicted immunogenicity may further be based on predictions and/or experimental indications of one or more immunogenicity factors. Factors that dictate immunogenicity can include one or more of: (i) a protein level of a mutant-peptide precursor; (ii) an expression level of a transcript encoding the mutant-peptide precursor; (iii) a processing efficiency of the mutant-peptide precursor by the immunoproteasome; (iv) the timing of the expression of the transcript encoding the mutant-peptide precursor; (v) a binding affinity of the mutant peptide to a T-cell receptor; (vi) a position of a variant amino acid within the mutant peptide; (vii) solvent exposure of the mutant peptide when bound to a MHC molecule; (vii) solvent exposure of the variant amino acid when bound to a MHC molecule; (x) the content of aromatic residues in the peptide; (xi) properties of the variant amino acid when compared to a wild type residue; (xii) the nature of the mutant-peptide precursor; (xiii) microbial similarity of the mutant peptide to known microbial peptides; (xiv) self-similarity or dissimilarity of the mutant peptide to the wild type proteome; or (xv) thymic expression of the wild type peptide. Immunogenicity factors can further or alternatively include a protein sequence of a mutant peptide, the length of a mutant peptide (e.g., as indicating by a number of amino acids identified within the variant-coding sequence), and/or an expression level of an MHC allotype in the subject (e.g., as measured by RNA-Seq or mass spectrometry).

Binding affinity predictions and/or predictions as to whether (or a probability that) mutant-peptide presentation will occur (e.g., by one or more tumor cells and/or one or more MHC molecules in the subject) can be generated in accordance with techniques disclosed herein (e.g., using an attention-based machine-learning model) for each of a set of mutant peptides (e.g., that were detected within a disease sample from a subject). These predictions can be used to select an incomplete subset of the set (e.g., less than 50% of the set, less than 25% of the set, less than 10% of the set, less than 5% of the set, and/or less than 1% of the set). The incomplete subset can be selected using one or more relative thresholds (e.g., to identify mutant peptides within the set that have the most stable bounds with MHC molecules and/or the highest likelihoods of being presented relative to others in the group) or one or more absolute thresholds. For example, each selected mutant peptide can have a binding affinity with MHC with a relatively strong affinity value (e.g., within a best 50%, best 25%, best 10% or best 5% affinity values within the set) and/or absolutely strong affinity value (e.g., having an affinity value of better than a predetermined threshold/cutoff, such as 5000 nM, 1000 nM, or 500 nM). The incomplete subset of the set may include 1, 2, 3, 4, 5, 6, 7, 8, 9, 10, or more mutant peptides irrespective of the predetermined affinity value threshold/cutoff. The incomplete subset of the set may include 20 or more neoantigens or 30 or more mutant peptides.

In some instances, a machine-learning model generates predictions corresponding to one or more potential interactions between a mutant peptide and an MHC molecule. For example, the machine-learning model may predict binding affinity of the MHC molecule and a mutant peptide. Additionally or alternatively, the machine-learning model may predict whether an MHC molecule will present the mutant peptide. The machine-learning model may receive, as input, and may process (e.g., using one or more processing layers) a sequence or subsequence of the MHC molecule and the variant-coding sequence associated with the mutant peptide.

In some instances, a machine-learning model generates predictions corresponding to one or more potential interactions between a mutant peptide, an MHC sequence or subsequence, and a T-cell receptor (e.g., instead of, or in addition to, generating predictions corresponding to one or more potential interactions between a mutant peptide and an MHC molecule). The machine-learning model may then predict, for example, a binding affinity between the mutant peptide and T-cell receptor and/or whether the mutant peptide activates and/or triggers an immunological response in the T cell. The machine-learning model may receive, as input, and may process (e.g., using one or more self-attention layers) a sequence or subsequence of the T-cell receptor, a sequence or subsequence of MHC, and the variant-coding sequence of the mutant peptide

A prediction generated in association with a mutant peptide can be, for example, numeric (e.g., corresponding to a predicted probability that an MHC molecule of the subject presents the mutant peptide at a cell surface or a predicted fraction of tumor cells in the subject that present the mutant peptide), categorical (e.g., predicting no, infrequent or frequent presentation of the mutant peptide by MHC molecules of the subject) or binary (e.g., predicting whether the mutant peptide is expressed by MHC molecules in the subject). A presentation prediction may (but need not) be normalized and/or represent a conditioned prediction. For example, a presentation prediction may correspond to a prediction as to whether an MHC molecule of the subject presents the mutant peptide if the mutant peptide has stably bound to the MHC molecule.

### Example Identification of Input Data for Machine-Learning Model

The example methods and systems for identifying input data described herein can be used to identify input data for, for example, machine-learning model 132 in FIGS. 1 and 3, any workflow in FIGS. 4A-D, and/or machine-learning model 532 described in FIGS. 5A-5C.

Each of a set of mutant peptides associated with a given subject can be analyzed using a machine-learning model to generate one or more predictions as to a binding affinity, presentation probability, and/or immunogenicity of a mutant peptide. To generate these predictions, the machine-learning model can receive and process a peptide (e.g., coding) sequence corresponding to the mutant peptide and one or more other sequences or subsequences (e.g., corresponding to an MHC-I molecule, an MHC-II molecule, or a T-cell receptor). In some instances, predictions are generated for each of a set of peptide sequences (e.g., a set of variant-coding sequences corresponding to a set of mutant peptides). The set of mutant peptides can correspond to peptides present in a disease sample collected from the subject but that are not observed in one or more non-disease samples (e.g., from the subject or another subject).

A variety of methods are available for identifying a set of mutant peptides associated with a given subject. Mutations can be present in the genome, transcription, proteome, or exome of diseased cells of a subject but not in a non-diseased sample, for example, a non-diseased sample from the subject or from another subject. Mutations include, but are not limited to: (1) non-synonymous mutations leading to different amino acids in the protein; (2) read-through mutations in which a stop codon is modified or deleted, leading to translation of a longer protein with a novel tumor-specific sequence at the C-terminus; (3) splice site mutations that lead to the inclusion of an intron in the mature mRNA and thus a unique tumor-specific protein sequence; (4) chromosomal rearrangements that give rise to a chimeric protein with tumor-specific sequences at the junction of 2 proteins (i.e., gene fusion); (5) frameshift insertions or deletions that lead to a new open reading frame with a novel tumor-specific protein sequence. Mutations can also include one or more of nonframeshift indel, missense or nonsense substitution, splice site alteration, genomic rearrangement or gene fusion, or any genomic or expression alteration giving rise to a neoORF.

Peptides with mutations or mutated polypeptides arising from, for example, splice-site, frameshift, readthrough, or gene fusion mutations in diseased cells can be identified by sequencing DNA, RNA, or protein in the diseased sample and comparing the obtained sequences with sequences from a non-diseased sample.

In some embodiments, whole genome sequencing (WGS) or whole exome sequencing (WES) data from a disease sample and a non-diseased sample can be obtained and compared. Following the alignment of non-diseased sample and diseased sample reads to the human reference genome, somatic variants, which include single nucleotide variants (SNV), gene fusions, and insertion or deletion variants (indels) can be detected using variant-calling algorithms. One or more variant callers can be used to detect different somatic variant types (e.g., SNV, gene fusions, or indels).

In some examples, the mutant peptides are identified based on the transcriptome sequences in the disease sample from the individual. For example, whole or partial transcriptome sequences can be obtained (for example, by methods such as RNA-Seq) from a diseased tissue of the individual and subjected to sequencing analysis. The sequences obtained from the diseased tissue sample can then be compared to those obtained from a reference sample. Optionally, the diseased tissue sample is subjected to whole-transcriptome RNA-Seq. Optionally, the transcriptome sequences are “enriched” for specific sequences prior to the comparison to a reference sample. For example, specific probes can be designed to enrich certain desired sequences (for example, disease-specific sequences) before being subjected to sequencing analysis.

In some embodiments, transcriptomic sequencing techniques include, but are not limited to, RNA poly(A) libraries, microarray analysis, parallel sequencing, massively parallel sequencing, PCR, and RNA-Seq. RNA-Seq is a high-throughput technique for sequencing part of, or substantially all of, the transcriptome. In short, an isolated population of transcriptomic sequences is converted to a library of cDNA fragments with adaptors attached to one or both ends. With or without amplification, each cDNA molecule is then analyzed to obtain short stretches of sequence information, typically 30-400 base pairs. These fragments of sequence information are then aligned to a reference genome, reference transcripts, or assembled de novo to reveal the structure of transcripts (e.g., transcription boundaries) and/or the level of expression.

Once obtained, the sequences in the diseased sample can be compared to the corresponding sequences in a reference sample. The sequence comparison can be conducted at the nucleic acid level, by aligning the nucleic acid sequences in the disease tissue with the corresponding sequences in a reference sample. Genetic sequence variations that lead to one or more changes in the encoded amino acids are then identified. Alternatively, the sequence comparison can be conducted at the amino acid level, that is, the nucleic acid sequences are first converted into amino acid sequences in silico before the comparison is carried out. Either the amino acid-based approach or the nucleic acid-based approach can be used to identify one or more mutations (e.g., one or more point mutations) in the peptide. With regard to nucleic acid-based approaches, the discovered variants can be used to identify one or more nucleic acid sequences (e.g., DNA sequences, RNA sequences, or mRNA sequences) that would give rise to a given observable mutant protein (e.g., via a look-up table associated individual peptide mutations with multiple codon variants).

In some embodiments, comparison of a sequence from the disease sample to those of a reference sample can be completed by techniques, such as manual alignment, FAST-All (FASTA), or Basic Local Alignment Search Tool (BLAST). In some embodiments, a comparison of a sequence from a disease sample to those of a reference sample can be completed using a short read aligner, for example, GSNAP, BWA, or STAR.

In some embodiments, the reference sample is a matched, disease-free sample. As used herein, a “matched,” disease-free tissue sample is one that is selected from the same or similar sample, for example, a sample from the same or similar tissue type as the disease sample. In some embodiments, a matched, disease-free tissue and a disease tissue may originate from the same individual. The reference sample described herein can be a disease-free sample from the same individual. In some embodiments, the reference sample is a disease-free sample from a different individual (for example, an individual not having the disease). In some embodiments, the reference sample is obtained from a population of different individuals. In some embodiments, the reference sample is a database of known genes associated with an organism. In some embodiments, a reference sample can be from a cell line. In some embodiments, a reference sample can be a combination of known genes associated with an organism and genomic information from a matched disease-free sample. In some embodiments, a variant-coding sequence may comprise a point mutation in the amino acid sequence. In some embodiments, the variant-coding sequence may comprise an amino acid deletion or insertion.

In some embodiments, the set of variant-coding sequences is first identified based on genomic and/or nucleic acid sequences. This initial set is then further filtered to obtain a narrower set of expression variant-coding sequences based on the presence of the variant-coding sequences in a transcriptome sequencing database (and is thus deemed “expressed”). In some embodiments, the set of variant-coding sequences are reduced by at least about 10, 20, 30, 40, 50, or more times by filtering through a transcriptome sequencing database.

Alternatively, protein mass spectrometry can be used to identify or validate the presence of mutant peptides, for example, mutant bound to MHC proteins on tumor cells. Peptides can be acid-eluted from diseased cell, for example, tumor cells or from HLA molecules that are immunoprecipitated from the tumor, and then identified using mass spectrometry.

A mutant peptide can have, for example, 5 or more, 8 or more, 11 or more, 15 or more, 20 or more, 40 or more, 80 or more, 100 or more, 120 or fewer, 100 or fewer, 80 or fewer, 60 or fewer, 50 or fewer, 40 or fewer, 30 or fewer, 25 or fewer, 20 or fewer, 18 or fewer, 15 or fewer, or 13 or fewer amino acids.

Tumor-specific T-cell receptor sequences can also be identified, for example, by single cell T-cell receptor sequencing. High-throughput sequencing of T-cell repertoires can also or alternatively be performed to identify tumor-specific signatures for a particular disease. MHC-I sequences and/or MHC-II sequences can be determined, for example, via HLA genotyping or mass spectroscopy.

### Example Identification of Training Data for Machine-Learning Model

The example methods and systems for identifying training data described herein can be used to identify training data for, for example, machine-learning model 132 in FIGS. 1 and 3, any workflow in FIGS. 4A-D, and/or machine-learning model 532 described in FIGS. 5A-5C. For example, these methods and systems can be used to identify training data 133 in FIG. 1.

A training set can be generated using data collected from multiple other samples (e.g., potentially being associated with one or more other subjects). Each of the multiple other samples can include, for example, tissue (e.g., a biopsy), single cell, multiple cells, fragments of cells, or an aliquot of body fluid. In some instances, the samples are collected from a different type of subject as compared to a subject associated with input data to be processed by the trained model. For example, a machine-learning model can be trained using training data collected by processing samples from one or more cell lines, and the trained machine-learning model can be used to process input data determined by processing one or more samples from a human subject.

The training data set can include multiple training elements. Each of the multiple training elements can include input data that includes a set of peptide sequences (which includes a set of either wild-type or variant-coding sequences), each of which code for and/or represent any variant in a corresponding peptide, and a subsequence or pseudosequence of an MHC molecule. The input data can be collected in accordance with one or more techniques disclosed herein.

Each training element can also include one or more experiment-based results. An experiment-based result can indicate whether and/or an extent to which each of one or more particular types of interaction between a wild-type peptide or mutant peptide (associated with a variant-coding sequence in the training element) and an MHC molecule (associated with an MHC molecule subsequence in the training element) occurs. A particular type of interaction can include, for example, binding of a peptide to an MHC molecule and/or presentation of a peptide by the MHC molecule on a surface of a cell (e.g., a tumor cell).

A result can include a binding affinity between the peptide and the MHC molecule. The result can include or can be based on qualitative data and/or quantitative data characterizing whether a given peptide binds with a given MHC molecule, the strength of such a bond, the stability of such a bond, and/or the tendency of such a bond to occur. For example, a binary binding-affinity indicator or a qualitative binary-affinity result can be generated using an ELISA, pull-down assay, gel-shift assay, biosensor-based methodology, such as Surface Plasmon Resonance, Isothermal Titration Colorimetry, BioLayer Interferometry, or MicroScale Thermophoresis.

The result can, for example, further or alternatively characterize whether and/or a probability that a given MHC molecule presents a given peptide. MHC ligands can be immunoprecipitated out of a sample. Subsequent elution and mass spectrometry can be used to determine whether the MHC molecule presented the ligand.

### Example Training Data Filtering

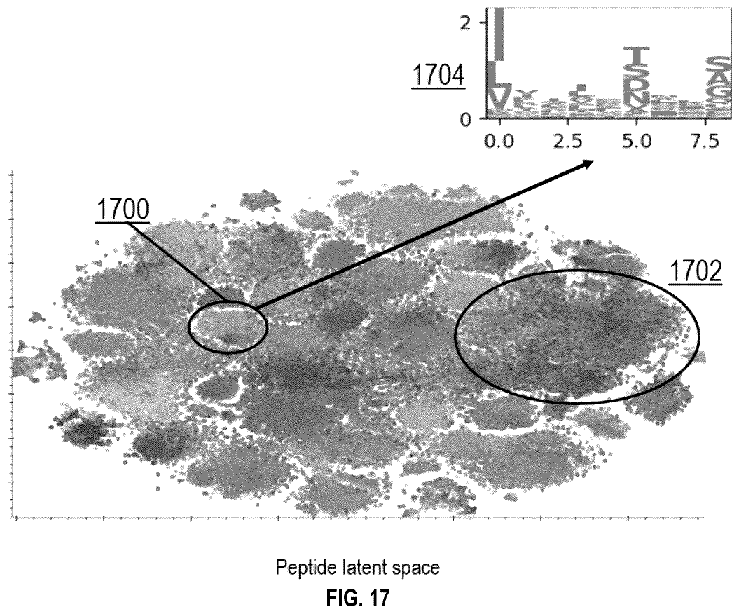

FIG. 17 illustrates a plot of a latent space that includes a plurality of peptide vectors, in accordance with some embodiments. Each peptide vector in FIG. 17 corresponds to a BOS token embedding of a peptide sequence (e.g., a BOS token embedding 408a in FIG. 4A) in a given sample. In some examples, a sample refers to a row in a dataset and the row specifies a peptide, a MHC, a TCR, or a combination thereof. In the latent space, each peptide vector has been reduced to two dimensions (e.g., using any of the dimensionality reduction techniques described herein) and is plotted as a single dot. The color of each dot represents the allele that the peptide is binding to.

As shown in FIG. 17, peptides having the same color (i.e., binding to the same allele) generally are close to each other in the latent space and thus form a cluster such as cluster 1700. However, in area 1702, many dots having the same color appear relatively scattered and do not form a clear cluster. The scattered distribution may indicate experimental error in the peptide vector data corresponding to the area 1702, as peptides associated with various random alleles should generally not occupy the same area in the latent space.

The above-referenced experimental errors can be identified and excluded from the training data (e.g., training data for the processing block 314) described herein. Specifically, the system (e.g., the computing platform 102 in FIG. 1) can identify, for a given peptide, K nearest neighbors to the peptide in the latent space and generate a motif. For example, a motif can be generated by calculating the probability of each amino acid at each position in a peptide, given a group of peptides with the same length (i.e., the K nearest neighbors), and converting the probability information to information entropy in bits. In other words, each motif indicates, at a given position in the peptide, a probability that an amino acid occurs. In some embodiments, an information content metric can be extracted for each peptide based on the motif to quantify whether a position is associated with a pattern of amino acid occurrence or not (which can be indicative of experimental errors). In some embodiments, the information content is calculated based on the number of bits of information in the max 2 positions. In some embodiments, the data associated with low information content can be filtered out from the training data.

FIG. 17 illustrates an example motif 1704 corresponding to K nearest neighbors in the cluster 1700. As shown in the motif 1704, at position 0 (x axis), much of the space is occupied by I and V, indicating that I and V occur frequently at this position. In contrast, at position 1 or position 2 (x axis), there is no single amino acid that occupies significantly more space than others. Accordingly, position 0 is associated with high information content, while positions 1 and 2 are associated with low information content due to the lack of pattern in the binding. An information content of the peptide can then be determined in bits accordingly. In some examples, a positional weight matrix (PWM) of a peptide with the nearest neighboring peptides is determined and the information content is calculated with KL divergence with respect to a baseline PWM calculated from the human peptidome. In some examples, a Shannon entropy is calculated as the information content.

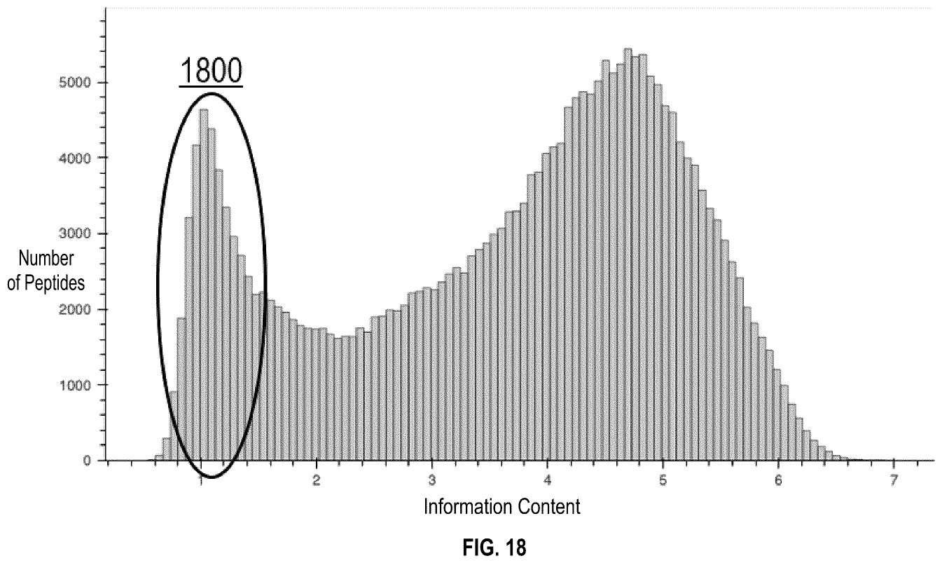

FIG. 18 illustrates a histogram showing the counts of peptides having different levels of information content, in accordance with some embodiments. In some examples, the input data comprises a dataset of peptides and HMCs. For each peptide, an information content is calculated (e.g., based on the respective peptides and neighboring peptides as described herein). The X-axis refers to information content of a peptide, which can be quantified by bits. Specifically, for a peptide, the system (e.g., the computing platform 102 in FIG. 1) can identify K nearest neighbors to the peptide in the latent space and generate a motif. For example, a motif can be generated by calculating the probability of each amino acid at each position in a peptide, given a group of peptides with the same length (i.e., the K nearest neighbors), and converting the probability information to information entropy (in bits) as the information content of the peptide. The Y-axis refers to the number of peptides having a specific level of information content. The histogram shows a large number of peptides (i.e., 1800) having relatively low information content in the dataset. Low information content indicates a lack of pattern in the binding (i.e., all positions in the peptide are equally random) and may be indicative of experimental errors in the dataset. Accordingly, those peptides having relatively low information content (e.g., below a threshold) can be filtered out from the training data.

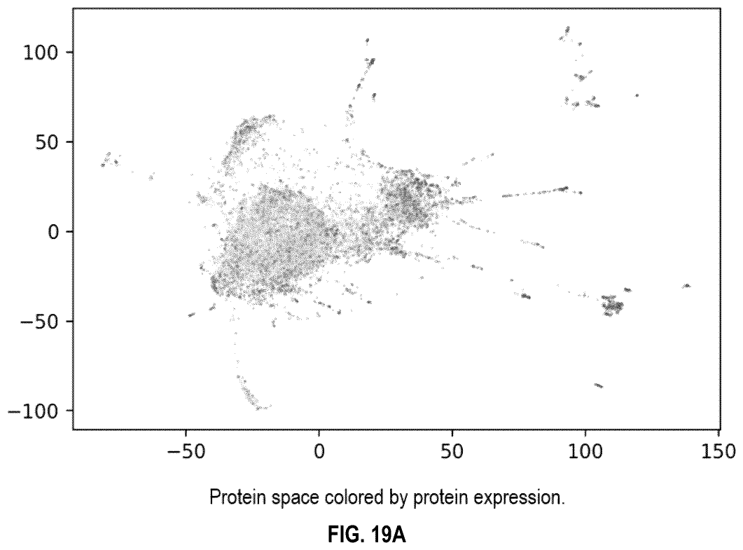

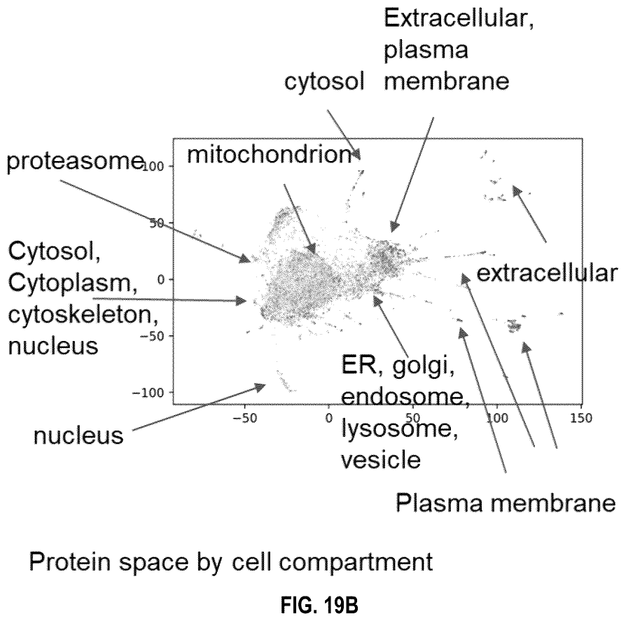

FIG. 19A illustrates a protein space colored by protein expression, in accordance with some embodiments. In FIG. 19A, each dot represents a protein vector (e.g., a dimensionally reduced version of the protein sequence embedding 422 in FIG. 4B). Further, blue indicates a lower expression protein, whereas red indicates a higher expression protein. As shown in FIG. 19A, there is a continuous gradient in the main cluster from blue to red. FIG. 19A demonstrates that, although the protein language model (e.g., PLM 444) is not trained using explicit protein expression data, the model nevertheless learns this representation of the proteins. FIG. 19B illustrates the cellular compartmentalization of different proteins and where they appeared in the latent space. FIG. 19B shows how the techniques disclosed herein can predict the source protein space/location by cell compartment. In some instances, it is desirable to predict a peptide that binds to MHC I, and such binding and presentation can happen more often when the peptide is derived from a source protein located within the cell. Conversely, in some other instances it is desirable to predict a peptide that binds to MHC II, and such binding and presentation can happen more often when the peptide is derived from a source protein that is primarily extracellular.

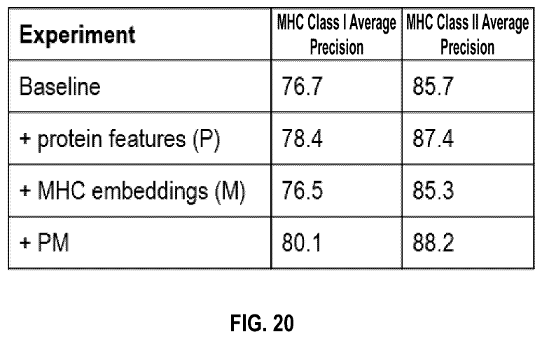

FIG. 20 illustrates example performance data, in accordance with some embodiments. Specifically, FIG. 20 shows the average precision values of a baseline algorithm for an MHC class I dataset and an MHC class II dataset. The baseline algorithm does not incorporate the processing of protein data (e.g., the processing of protein sequence embedding 422 in FIG. 4B) or incorporate the processing of MHC data (e.g., the processing of the MHC sequence embedding 426 in FIG. 4B). Instead, the baseline algorithm uses one or more transformer stages to use a BOS token-appended MHC sequence to generate a BOS+MHC sequence representations and then use one or more transformer stages to generate a transformed BOS+MHC sequence representation (e.g., the processing of BOS token-appended MHC sequence in FIG. 4A). As shown in FIG. 20, the incorporation of protein information (e.g., the processing of protein sequence embedding 422 in FIG. 4B), the incorporation of the MHC sequence embedding (e.g., the processing of the MHC sequence embedding 426 in FIG. 4B), and the incorporation of both (e.g., the workflow 420 in FIG. 4B, which incorporates both the processing of protein sequence embedding 422 and the processing of the MHC sequence embedding 426) improve the performance of the baseline algorithm.

### Example Pharmaceutically-Acceptable Compositions

In some embodiments, for each of a set of mutant peptides (e.g., detected in a sample of a subject), one or more techniques disclosed herein are used to predict whether the mutant peptide will bind to a subject's MHC molecule (or a strength, stability, and/or prevalence of such binding) and/or to predict whether a subject's MHC molecule will present the mutant peptide (and/or a prevalence of such presentation). The predictions can be used to select an incomplete subset of the mutant peptides (e.g., for which it is predicted that MHC presentation of the mutant peptide is likely). The selection may include comparing, for each mutant peptide, a metric corresponding to the prediction metric to an absolute threshold and/or to prediction metrics of other mutant peptides' metrics (e.g., thereby performing a relative comparison). Each selected mutant peptide can be identified as having one or more of: a high likelihood of being presented on the tumor cell surface, a high likelihood of being capable of inducing a tumor-specific immune response, a high likelihood of being capable of being presented to naive T cells by antigen presenting cells (e.g., dendritic cells), a low likelihood of being subject to inhibition via central or peripheral tolerance, or a low likelihood of being capable of inducing an autoimmune response to normal tissue in the subject.

As one non-limiting example, a selection can include identifying each of the set of subject-specific set of variant-coding sequences for which a predicted binding affinity is less than 500 nM, for which it is predicted that an MHC molecule will present a mutant peptide identified by the variant-coding sequence and/or for which it is predicted that the mutant peptide will trigger an immune response. It will be appreciated that outputs of the model can be on a different scale, such that 500 nM may correspond to, for example, another value (e.g., 0.42) on a [0,1] scale.

Each selected mutant peptide can be manufactured, experimentally tested (e.g., to determine a binding affinity, presentation prevalence, and/or other immunological factor), included in a composition (e.g., a pharmaceutical composition, such as a vaccine and/or treatment), and/or administered to a subject.

Each of the set of mutant peptides for which binding-affinity and presentation predictions are generated may include a mutant peptide associated with a particular subject (e.g., a particular human subject). Each of the set of mutant peptides can be a disease-specific, immunogenic mutant peptide identified using a disease-specific sample from an individual. The individual variant-coding sequence can be identified by sequencing genetic and/or nucleic acid sequences (e.g., DNA, RNA, and/or mRNA sequences) in a disease sample and comparing each identified genetic and/or nucleic acid sequence to a reference-sample sequence. Codons within a genetic and/or nucleic acid sequence are indicative of the existence of a corresponding amino acid in a peptide. Notably, each of multiple codons may encode a given amino acid, so while a nucleic acid sequence can indicate (e.g., deterministically) an amino acid sequence, the same amino acid sequence can be encoded by other nucleic acid sequences.

Some embodiments include manufacturing a composition based on one or more selected mutant peptides (or a plurality of nucleic acids encoding the one or more selected mutant peptides). For example, each of the one or more selected mutant peptides may have been predicted to bind to and be presented by an MHC molecule of the subject (e.g., at least to a threshold degree). The composition may include each of the one or more selected mutant peptides, one or more precursors to the one or more selected mutant peptides, one or more polypeptide sequences corresponding to the one or more selected mutant peptides, RNA (e.g., mRNA) corresponding to the one or more selected mutant peptides, DNA corresponding to the one or more selected mutant peptides, cells (e.g., antigen-presenting cells) including the one or more selected mutant peptides and/or nucleic acid(s) encoding such peptides, plasmids corresponding to the one or more selected mutant peptides, and/or vectors corresponding to the one or more selected mutant peptides.

The composition may include mutant peptides corresponding to a single selected variant-coding sequence. The composition may include mutant peptides and/or mutant-peptide precursors corresponding to multiple selected variant-coding sequences. A subset of peptide candidates (e.g., associated with the 5, 10, 15, 20, 30, or any number in between, highest presentation predictions) can be used for further precursor development.

Each of one or more (e.g., all) of the mutant peptides in the composition can have, for example, a length of about 7 to about 40 amino acids (e.g., about any of 7, 8, 9, 10, 11, 12, 13, 14, 15, 17, 20, 22, 25, 30, 35, 40, 45, 50, 60, or 70 amino acids in length). In some embodiments, a length of each of one or more (e.g., all) of the mutant peptides in the composition are within a predetermined range (e.g., 8-11 amino acids, 8-12 amino acids, or 8 to 15 amino acids). In some embodiments, each of one or more (e.g., all) of the mutant peptides in the composition is about 8-10 amino acids in length. Each of one or more (e.g., all) of the mutant peptides in the compositions can be in its isolated form. Each of one or more (e.g., all) of the mutant peptides in the composition can be a “long peptide” produced by adding one or more peptides to an end (or to each end) of the mutant peptide. Each of one or more (e.g., all) of the mutant peptides in the composition can be tagged, a fusion protein, and/or a hybrid molecule.

In some embodiments, the composition can be developed by using one or more nucleic acids that encode the peptide. The nucleic acid(s) can include DNA, RNA, and/or mRNA. Given that any of multiple codons can encode a given amino acid, the codons can be selected to, for example, optimize or promote expression in a given type of organism. Such selection can be based on the frequency that each of multiple potential codons are used by the given type of organism, the translational efficiency of each of multiple potential codons in the given type of organism, and/or the given type of organism's degree of bias towards each of the multiple potential codons.

The composition may include a polynucleotide construct (e.g., a DNA construct or an RNA construct). The polynucleotide construct is an artificially constructed segment of nucleic acid which can be ‘transplanted’ into a target tissue or cell. The polynucleotide construct comprises a DNA or RNA (e.g., mRNA) insert, which contains the nucleotide sequence encoding the one or more selected mutant peptides. In order to increase antigen presentation (e.g., presentation of the one or more selected mutant peptides by a MHC molecule), the polynucleotide construct may further comprise a modification developed for improved antigen presentation, and thus improved immunogenicity to the one or more selected mutant peptides. In some instances, the modification is incorporation of a transmembrane region and a cytoplasmic region of a chain of the MHC molecule into the polynucleotide construct.

To provide an RNA insert with increased stability and translation efficiency, the polynucleotide construct may further comprise a modification developed for improved stability and translation, and thus improved immunogenicity to the one or more selected mutant peptides. In some instances, the modification includes incorporation of a nucleic acid sequence with at least two copies of a 3′-untranslated region of a human beta-globin gene into the polynucleotide construct. In some instances, the modification includes incorporation of a nucleic acid sequence that codes for a 3′-untranslated region such as F1 3′ UTR.

In some instances, the composition may include nucleic acids encoding the mutant peptide(s) or precursor of the mutant peptide(s) described above. The nucleic acid may include sequences flanking the sequence coding the mutant peptide (or precursor thereof). In some instances, the nucleic acid includes epitopes corresponding to more than one selected variant-coding sequence. In some instances, the nucleic acid is DNA having a polynucleotide sequence encoding the mutant peptides or precursors described above.

In some instances, the nucleic acid is RNA. In some instances, the RNA is reverse transcribed from a DNA template having a polynucleotide sequence encoding the mutant peptides or precursors described above. In some instances, the RNA is mRNA. In some instances, the RNA is naked mRNA. In some instances, the RNA is modified mRNA (e.g., mRNA protected from degradation using protamine mRNA containing modified 5′CAP structure, or mRNA containing modified nucleotides). In some embodiments, the RNA is single-stranded mRNA.

To provide an RNA insert with increased stability and expression, the polynucleotide construct may further comprise a modification developed for improved stability and expression, and thus improved immunogenicity to the one or more selected mutant peptides. In some instances, the modification is incorporation of a cap on an end of the RNA such as a 5′-cap structure. The cap structure can be the D1 diastereomer of beta-S-ARCA.

In order to deliver the polynucleotide construct with high selectivity to antigen presenting cells, the composition may further include cationic liposomes or a lipoplex for improved uptake of the polynucleotide construct, and thus improved immunogenicity to the one or more selected mutant peptides. In some instances, the composition includes nanoparticles comprising the polynucleotide construct. The nanoparticles can be lipoplexes comprising one or more lipids such as DOTMA and DOPE.

The composition may include cells comprising the mutant peptide and/or nucleic acid(s) encoding the mutant peptide described above. The composition may further comprise one or more suitable vectors and/or one or more delivery systems for the mutant peptide. In some embodiments, the composition may comprise nucleic acid(s) encoding the mutant peptide. In some instances, the cells comprising the mutant peptide and/or nucleic acids encoding the mutant peptide are non-human cells, for example, bacterial cells, protozoan cells, fungal cells, or non-human animal cells. In some instances, the cells comprising the mutant peptide and/or nucleic acids encoding the mutant peptide are human cells. In some instances, the human cells are immune cells. In some instances, the immune cells are antigen-presenting cells (APCs). In some instances, the APCs are professional APCs, such as macrophages, monocyte, dendritic cells, B cells, and microglia. In other instances, the professional APCs are macrophages or dendritic cells. In some instances, the APCs comprising the mutant peptide and/or nucleic acid sequence(s) encoding the mutant peptide are used as a cellular vaccine, thereby inducing a CD4+ or a CD8+ immune response. In other instances, the composition used as a cellular vaccine includes mutant peptide-specific T cells primed by APCs comprising the mutant peptide and/or nucleic acid sequence(s) encoding the mutant peptide.

The composition may include a pharmaceutically-acceptable adjuvant, pharmaceutically-acceptable excipient, an immunomodulator, a checkpoint protein, an antagonist of PD-1 (e.g., an anti-PD-1 antibody), and/or an antagonist of PD-L1 (e.g., an anti-PD-L1 antibody). Adjuvants refer to any substance for which admixture into a composition modifies an immune response to a mutant peptide. Adjuvants can be conjugated using, for example, an immune stimulation agent. Excipients can increase the molecular weight of a particular mutant peptide to increase activity or immunogenicity, confer stability, increase biological activity, and/or increase serum half-life.

The pharmaceutically-acceptable composition can be a vaccine, which can include an individualized vaccine that is specific to (e.g., and potentially developed for) a particular subject. For example, an MHC sequence may have been identified using a sample from the particular subject, and the composition can be developed for and/or used to treat the particular subject.

The vaccine can be a nucleic acid vaccine. The nucleic acid can encode a mutant peptide or precursor of the mutant peptide. The nucleic acid vaccine may include sequences flanking the sequence coding the mutant peptide (or precursor thereof). In some instances, the nucleic acid vaccine includes epitopes corresponding to more than one selected variant-coding sequence. In some instances, the nucleic acid vaccine is a DNA-based vaccine. In some instances, the nucleic acid vaccine is an RNA-based vaccine. In some instances, the RNA-based vaccine comprises mRNA. In some instances, the RNA-based vaccine comprises naked mRNA. In some instances, the RNA-based vaccine comprises modified mRNA (e.g., mRNA protected from degradation using protamine mRNA containing modified 5′CAP structure, or mRNA containing modified nucleotides). In some embodiments, the RNA-based vaccine comprises single-stranded mRNA.

A nucleic acid vaccine may include an individualized neoantigen specific therapy manufactured for a particular subject to be used as part of next-generation immunotherapy. The individualized vaccine may have been designed by first detecting mutant peptides in a sample of the particular subject and subsequently predicting, for each detected mutant peptide, whether and/or a degree to which the peptide will bind to an MHC of the particular subject, be presented by the MHC, bind to a T-cell receptor of the particular subject, and/or trigger an immunological response. Based on these predictions, a subset of the detected mutant peptides can be selected (e.g., a subset having at least 1, at least 2, at least 3, at least 5, at least 8, at least 10, at least 12, at least 15, at least 18, up to 40, up to 30, up to 25, up to 20, up to 18, up to 15, and/or up to 10 mutant peptides). For each selected mutant peptide, a synthetic mRNA sequence can be identified that codes for the mutant peptide. An mRNA vaccine may include mRNA (that encodes part or all of a mutant peptide) complexed with lipids to form an mRNA-lipoplex. Administration of a vaccine that includes the mRNA-lipoplex can result in the mRNA stimulating TLR7 and TLR8, triggering T cell activation by dendritic cells. Further, the administration can result in translation of mRNA into a mutant peptide, which can then bind to and be presented by MHC molecules and induce T cell response.

The composition may include substantially pure mutant peptides, substantially pure precursors thereof, and/or substantially pure nucleic acids encoding the mutant peptides or precursors thereof. The composition may include on more suitable vectors and/or one or more delivery systems to contain the mutant peptides, precursors thereof, and/or nucleic acids encoding the mutant peptides or precursors thereof. Suitable vectors and delivery systems include viral, such as systems based on adenovirus, vaccinia virus, retroviruses, herpes virus, adeno-associated virus, or hybrids containing elements of more than one virus. Non-viral delivery systems include cationic lipids and cationic polymers (e.g., cationic liposomes). In some embodiments, physical delivery, such as with a ‘gene-gun’ can be used.

In certain embodiments, the RNA-based vaccine includes an RNA molecule including, in 5′→3′ direction: (1) a 5′ cap; (2) a 5′ untranslated region (UTR); (3) a polynucleotide sequence encoding a secretory signal peptide; (4) a polynucleotide sequence encoding the one or more mutant peptides resulting from cancer-specific somatic mutations present in the tumor specimen; (5) a polynucleotide sequence encoding at least a portion of a transmembrane and cytoplasmic domain of a major histocompatibility complex (MHC) molecule; (6) a 3′ UTR including: (a) a 3′ untranslated region of an Amino-Terminal Enhancer of Split (AES) mRNA or a fragment thereof; and (b) non-coding RNA of a mitochondrially-encoded 12S RNA or a fragment thereof; and (7) a poly(A) sequence.

In certain embodiments, the RNA molecule further includes a polynucleotide sequence encoding an amino acid linker; wherein the polynucleotide sequences encoding the amino acid linker and a first of the one or more mutant peptides form a first linker-neoepitope module; and wherein the polynucleotide sequences forming the first linker-neoepitope module are between the polynucleotide sequence encoding the secretory signal peptide and the polynucleotide sequence encoding the at least portion of the transmembrane and cytoplasmic domain of the MHC molecule in 5′→3′ direction. In certain embodiments, the amino acid linker includes the sequence GGSGGGGSGG (SEQ ID NO: 1). In certain embodiments, the polynucleotide sequence encoding the amino acid linker includes the sequence GGCGGCUCUGGAGGAGGCGGCUCCGGAGGC (SEQ ID NO: 2).

In certain embodiments, the RNA molecule further includes, in 5′→3′ direction: at least a second linker-epitope module, wherein the at least second linker-epitope module includes a polynucleotide sequence encoding an amino acid linker and a polynucleotide sequence encoding a neoepitope; wherein the polynucleotide sequences forming the second linker-neoepitope module are between the polynucleotide sequence encoding the neoepitope of the first linker-neoepitope module and the polynucleotide sequence encoding the at least portion of the transmembrane and cytoplasmic domain of the MHC molecule in 5′→3′ direction; and wherein the neoepitope of the first linker-epitope module is different from the neoepitope of the second linker-epitope module. In certain embodiments, the RNA molecule includes 5 linker-epitope modules, wherein the 5 linker-epitope modules each encode a different neoepitope. In certain embodiments, the RNA molecule includes 10 linker-epitope modules, wherein the 10 linker-epitope modules each encode a different neoepitope. In certain embodiments, the RNA molecule includes 20 linker-epitope modules, wherein the 20 linker-epitope modules each encode a different neoepitope.

In certain embodiments, the RNA molecule further includes a second polynucleotide sequence encoding an amino acid linker, wherein the second polynucleotide sequence encoding the amino acid linker is between the polynucleotide sequence encoding the neoepitope that is most distal in 3′ direction and the polynucleotide sequence encoding the at least portion of the transmembrane and cytoplasmic domain of the MHC molecule.

In certain embodiments, 5′ cap includes a D1 diastereoisomer of the structure:

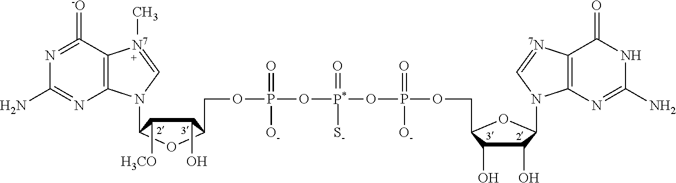

In certain embodiments, 5′ UTR includes the sequence UUCUUCUGGUCCCCACAGACUCAGAGAGAACCCGCCACC (SEQ ID NO: 3). In certain embodiments, 5′ UTR includes the sequence

(SEQ ID NO: 4)
GGCGAACUAGUAUUCUUCUGGUCCCCACAGACUCAGAGAGAACCCGCCAC
C.

In certain embodiments, the secretory signal peptide includes the amino acid sequence MRVMAPRTLILLLSGALALTETWAGS (SEQ ID NO: 5). In certain embodiments, the polynucleotide sequence encoding the secretory signal peptide includes the sequence

(SEQ ID NO: 6)
AUGAGAGUGAUGGCCCCCAGAACCCUGAUCCUGCUGCUGUCUGGCGCCCU
GGCCCUGACAGAGACAUGGGCCGGAAGC.

In certain embodiments, the at least portion of the transmembrane and cytoplasmic domain of the MHC molecule includes the amino acid sequence IVGIVAGLAVLAVVVIGAVVATVMCRRKSSGGKGGSYSQAASSDSAQGSDVSLTA (SEQ ID NO: 7). In certain embodiments, the polynucleotide sequence encoding the at least portion of the transmembrane and cytoplasmic domain of the MHC molecule includes the sequence

(SEQ ID NO: 8)
AUCGUGGGAAUUGUGGCAGGACUGGCAGUGCUGGCCGUGGUGGUGAUCGG
AGCCGUGGUGGCUACCGUGAUGUGCAGACGGAAGUCCAGCGGAGGCAAGG
GCGGCAGCUACAGCCAGGCCGCCAGCUCUGAUAGCGCCCAGGGCAGCGAC
GUGUCACUGACAGCC.

In certain embodiments, 3′ untranslated region of the AES mRNA includes the sequence CUGGUACUGCAUGCACGCAAUGCUAGCUGCCCCUUUCCCGUCCUGGGUACCCC GAGUCUCCCCCGACCUCGGGUCCCAGGUAUGCUCCCACCUCCACCUGCCCCACU CACCACCUCUGCUAGUUCCAGACACCUCC (SEQ ID NO: 9). In certain embodiments, the non-coding RNA of the mitochondrially-encoded 12S RNA includes the sequence CAAGCACGCAGCAAUGCAGCUCAAAACGCUUAGCCUAGCCACACCCCCACGGG AAACAGCAGUGAUUAACCUUUAGCAAUAAACGAAAGUUUAACUAAGCUAUAC UAACCCCAGGGUUGGUCAAUUUCGUGCCAGCCACACCG (SEQ ID NO: 10). In certain embodiments, 3′ UTR includes the sequence

(SEQ ID NO: 11)
CUCGAGCUGGUACUGCAUGCACGCAAUGCUAGCUGCCCCUUUCCCGUCCU
GGGUACCCCGAGUCUCCCCCGACCUCGGGUCCCAGGUAUGCUCCCACCUC
CACCUGCCCCACUCACCACCUCUGCUAGUUCCAGACACCUCCCAAGCACG
CAGCAAUGCAGCUCAAAACGCUUAGCCUAGCCACACCCCCACGGGAAACA
GCAGUGAUUAACCUUUAGCAAUAAACGAAAGUUUAACUAAGCUAUACUAA
CCCCAGGGUUGGUCAAUUUCGUGCCAGCCACACCGAGACCUGGUCCAGAG
UCGCUAGCCGCGUCGCU.

In certain embodiments, the poly(A) sequence includes 120 adenine nucleotides.

In certain embodiments, the RNA-based vaccine includes an RNA molecule including, in 5′→3′ direction: the polynucleotide sequence GGCGAACUAGUAUUCUUCUGGUCCCCACAGACUCAGAGAGAACCCGCCACCAU GAGAGUGAUGGCCCCCAGAACCCUGAUCCUGCUGCUGUCUGGCGCCCUGGCCC UGACAGAGACAUGGGCCGGAAGC (SEQ ID NO: 12); a polynucleotide sequence encoding the one or more mutant peptides resulting from cancer-specific somatic mutations present in the tumor specimen; and the polynucleotide sequence

(SEQ ID NO: 13)
AUCGUGGGAAUUGUGGCAGGACUGGCAGUGCUGGCCGUGGUGGUGAUCGG
AGCCGUGGUGGCUACCGUGAUGUGCAGACGGAAGUCCAGCGGAGGCAAGG
GCGGCAGCUACAGCCAGGCCGCCAGCUCUGAUAGCGCCCAGGGCAGCGAC
GUGUCACUGACAGCCUAGUAACUCGAGCUGGUACUGCAUGCACGCAAUGC
UAGCUGCCCCUUUCCCGUCCUGGGUACCCCGAGUCUCCCCCGACCUCGGG
UCCCAGGUAUGCUCCCACCUCCACCUGCCCCACUCACCACCUCUGCUAGU
UCCAGACACCUCCCAAGCACGCAGCAAUGCAGCUCAAAACGCUUAGCCUA
GCCACACCCCCACGGGAAACAGCAGUGAUUAACCUUUAGCAAUAAACGAA
AGUUUAACUAAGCUAUACUAACCCCAGGGUUGGUCAAUUUCGUGCCAGCC
ACACCGAGACCUGGUCCAGAGUCGCUAGCCGCGUCGCU.

In some embodiments, mutant peptides described herein (e.g., including or consisting of an ordered set of amino acids as identified by variant-coding sequences selected based on results from a machine-learning technique described herein) can be used for making mutant peptide specific therapeutics, such as antibody therapeutics. For example, the mutant peptides can be used to raise and/or identify antibodies specifically recognizing the mutant peptides. These antibodies can be used as therapeutics. Synthetic short peptides have been used to generate protein-reactive antibodies. An advantage of immunizing with synthetic peptides is that unlimited quantity of pure stable antigen can be used. This approach involves synthesizing the short peptide sequences, coupling them to a large carrier molecule, and immunizing a subject with the peptide-carrier molecule. The properties of antibodies are dependent on the primary sequence information. A good response to the desired peptide usually can be generated with careful selection of the sequence and coupling method. Most peptides can elicit a good response. An advantage of anti-peptide antibodies is that they can be prepared immediately after determining the amino acid sequence of a mutant peptide and particular regions of a protein can be targeted specifically for antibody production. Selecting mutant peptides for which a machine-learning model predicted immunogenicity and/or screening for the same can lead to a high chance that the resulting antibody will recognize the native protein in the tumor setting. A mutant peptide can be, for example, 15 or fewer, 18 or fewer or 20 or fewer, 25 or fewer, or 30 or fewer residues. A mutant peptide can be, for example, 9 or more, 10 or more, 15 or more, 20 or more, 25 or more, 30 or more, 50 or more, or 70 or more residues. Shorter peptides can improve antibody production.

Peptide-carrier protein coupling can be used to facilitate production of high titer antibodies. A coupling method can include, for example, site-directed coupling and/or a technique that relies on the reactive functional groups in amino acids, such as —NH2, —COOH, —SH, and phenolic —OH. Any suitable method used in anti-peptide antibody production can be utilized with the mutant peptides identified by the methods of the present invention. Two such known methods are the Multiple Antigenic Peptide system (MAPs) and the Lipid Core Peptides (LCP method). An advantage of MAPs is that the conjugation method is not necessary. No carrier protein or linkage bond is introduced into the immunized host. One disadvantage is that the purity of the peptide is more difficult to control. In addition, MAPs can bypass the immune response system in some hosts. The LCP method is known to provide higher titers than other anti-peptide vaccine systems and thus can be advantageous.

Also provided herein are isolated MHC/peptide complexes comprising one or more mutant peptides identified using a technique disclosed herein. Such MHC/peptide complexes can be used, for example, for identifying antibodies, soluble TCRs, or TCR analogs. One type of antibody has been termed TCR mimics, as they are antibodies that bind peptides from tumor associated antigens in the context of specific HLA environments. This type of antibody has been shown to mediate the lysis of cells expressing the complex on their surface as well as protect mice from implanted cancer cells lines that express the complex. One advantage of TCR mimics as IgG mAbs is that affinity maturation can be performed, and the molecules are coupled with immune effector functions through the present Fc domain. These antibodies can also be used to target therapeutic molecules to tumors, such as toxins, cytokines, or drug products.

Other types of molecules that have been developed using mutant peptides such as those selected using the methods of the present invention using non-hybridoma based antibody production or production of binding competent antibody fragments such as anti-peptide Fab molecules on bacteriophage. These fragments can also be conjugated to other therapeutic molecules for tumor delivery such as anti-peptide MHC Fab-immunotoxin conjugates, anti-peptide MHC Fab-cytokine conjugates, and anti-peptide MHC Fab-drug conjugates.

### Example Methods of Treatment

Some embodiments include treating a medical condition (e.g., tumor) or disease (e.g., cancer) in an individual by administering, to the individual, an effective amount of a composition (e.g., a vaccine) including one or more selected mutant peptides. The individual can be the same individual from whom a disease sample was collected. In some instances, the vaccine is administered to a different individual as compared to the individual from whom the disease sample was collected. The different individual may, for example, be related to the individual from whom the disease sample was collected, have a genetic risk of developing a particular type of cancer, and/or have MHC molecules that have one or more (e.g., all) alleles corresponding to a sequence that are the same (or similar) to one or more MHC alleles of the subject from who the disease sample was collected.

Some embodiments provide methods of treatment including a vaccine, which can be an immunogenic vaccine. In some embodiments, a method of treatment for disease (such as cancer) is provided, which may include administering to an individual an effective amount of a composition described herein, a mutant peptide identified using a technique disclosed herein, a precursor thereof, or nucleic acids encoding a mutant peptide (or precursor) identified using a technique described herein.

In some embodiments, a method of treatment for a disease (such as cancer) is provided. The method may include collecting a sample (e.g., a blood sample) from a subject. T cells can be isolated and stimulated. The isolation can be performed using, for example, density gradient sedimentation (e.g., and centrifugation), immunomagnetic selection, and/or antibody-complex filtering. The stimulation may include, for example, antigen-independent stimulation, which may use a mitogen (e.g., PHA or Con A), anti-CD3 antibodies (e.g., to bind to CD3 and activate the T-cell receptor complex), and/or anti-CD28 antibodies (e.g., to bind to CD28 and stimulate T cells). One or more mutant peptides can be (or may have been) selected for use in the treatment of the subject (e.g., based on results produced by a machine-learning model corresponding to predictions as to whether and/or an extent to which each of set of mutant peptides would bind to an MHC molecule of the individual, be presented by an MHC molecule of the individual, and/or trigger an immune response in the individual). The one or more mutant peptides may have been selected based on a technique disclosed herein that includes identifying and processing one or more sequence representations associated with the subject (e.g., a representation of: an MHC sequence, a set of variant-coding sequences, and/or a T-cell receptor sequence). The one or more sequences may have been detected using the sample from which the T cells were isolated or a different sample.

In some instances, the one or more mutant peptides (or precursors thereof) can be used to produce mutant peptide (for example, neoantigen) specific T cells. For example, peripheral blood T cells can be isolated from a subject and contacted with one or more mutant peptides to induce mutant peptide-specific T cells populations that can be administered to a subject. In some examples, the T-cell receptor sequence of the mutant peptide-reactive T cells can be sequenced. If the sequencing identifies an ordered set of nucleic acids, each codon of nucleic acids can be translated to an amino acid (e.g., via a look-up technique). Once a T-cell receptor sequence (e.g., amino acid T-cell receptor sequence) is obtained, T cells can be engineered to include the T-cell receptor that specifically recognizes the mutant peptide. These engineered T cells can then be administered to a subject. In any of the methods provided herein, the T cells can be expanded in vitro and/or ex vivo prior to administration to a subject. The subject may then be administered (e.g., infused with) a composition that includes the expanded population of T cells.

In some instances, a method of treatment for a disease (such as cancer) is provided, which may include administering to an individual a composition that includes one or more mutant peptides (or one or more precursors thereof) in an amount effective to, for example, prime, activate, and expand T cells in vivo.

In some embodiments, a method of treatment for a disease (such as cancer) is provided, which may include administering to an individual an effective amount of a composition including a precursor of a mutant peptide selected using a technique described herein. In some embodiments, an immunogenic vaccine may include a pharmaceutically-acceptable mutant peptide selected using a technique described herein. In some embodiments, an immunogenic vaccine may include a pharmaceutically-acceptable precursor to a mutant peptide selected using a technique described herein (such as a protein, peptide, DNA, and/or RNA). In some embodiments, a method of treatment for a disease (such as cancer) is provided, which may include administering to an individual an effective amount of an antibody specifically recognizing a mutant peptide selected using a technique described herein. In some embodiments, a method of treatment for a disease (such as cancer) is provided, which may include administering to an individual an effective amount of a soluble TCR or TCR analog specifically recognizing a mutant peptide selected using a technique described herein.

In some embodiments, the cancer is any one of: carcinoma, lymphoma, blastema, sarcoma, leukemia, squamous cell cancer, lung cancer (including small cell lung cancer, non-small cell lung cancer, adenocarcinoma of the lung, and squamous carcinoma of the lung), cancer of the peritoneum, hepatocellular cancer, gastric, or stomach (including gastrointestinal cancer), pancreatic cancer, glioblastoma, cervical cancer, ovarian cancer, liver cancer, bladder cancer, hepatoma, breast cancer, colon cancer, melanoma, endometrial or uterine carcinoma, salivary gland carcinoma, kidney or renal cancer, liver cancer, prostate cancer, vulval cancer, thyroid cancer, hepatic carcinoma, head and neck cancer, colorectal cancer, rectal cancer, soft-tissue sarcoma, Kaposi's sarcoma, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, Waldenstrom's macroglobulinemia, chronic lymphocytic leukemia (CLL), acute lymphoblastic leukemia (ALL), myeloma, Hairy cell leukemia, chronic myeloblasts leukemia, post-transplant lymphoproliferative disorder (PTLD), as well as abnormal vascular proliferation associated with phakomatoses, edema (such as that associated with brain tumors), or Meigs' syndrome.

Embodiments disclosed herein can including identifying part or all of and/or implementing part or all of an individualized-medicine strategy. For example, one or more mutant peptides can be selected for use in a vaccine by determining an MHC sequence and/or a set of variant-coding sequences using a sample from an individual, and processing representations of the MHC sequence and the variant-coding sequences using a machine-learning model disclosed herein (e.g., an attention-based machine-learning model). The one or more mutant peptides (and/or precursors thereof) may then be administered to the same individual.

In some embodiments, a method of treating a disease (such as cancer) in an individual is provided that includes: (a) identifying one or more mutant peptides in the individual (e.g., based on results produced by a machine-learning model corresponding to predictions as to whether and/or an extent to which each of set of mutant peptides would bind to an MHC molecule of the individual, be presented by an MHC molecule of the individual, and/or trigger an immune response in the individual, in accordance with one or more techniques disclosed herein); (b) synthesizing the identified mutant peptide(s) or one or more precursors of the mutant peptide(s) or nucleic acid(s) (e.g., polynucleotides such as DNA or RNA) encoding the identified peptide(s) or peptide precursor(s); and (c) administering the mutant peptide(s), mutant-peptide precursor(s), or nucleic acid(s) to the individual.

In some embodiments, the method of treating a disease (such as cancer) in an individual is provided that includes: (a) identifying one or more mutant peptides in the individual (e.g., based on results produced by a machine-learning model corresponding to predictions as to whether and/or an extent to which each of set of mutant peptides would bind to an MHC molecule of the individual, be presented by an MHC molecule of the individual and/or trigger an immune response in the individual, in accordance with one or more techniques disclosed herein); and (b) optionally, identifying a set of nucleic acids (e.g., polynucleotides such as DNA or RNA) that encode the identified mutant peptide(s) or one or more precursors of the mutant peptide(s), synthesizing the set of nucleic acids, and administering the set of nucleic acids to the individual.

In some embodiments, a method of treating a disease (such as cancer) in an individual is provided that includes: (a) identifying one or more mutant peptides in the individual (e.g., based on results produced by a machine-learning model corresponding to predictions as to whether and/or an extent to which each of set of mutant peptides would bind to an MHC molecule of the individual, be presented by an MHC molecule of the individual, and/or trigger an immune response in the individual, in accordance with one or more techniques disclosed herein); (b) producing an antibody specifically recognizing the mutant peptide; and (c) administering the peptide to the individual.

The methods provided herein can be used to treat an individual (e.g., human) who has been diagnosed with or is suspected of having cancer. In some embodiments, an individual can be a human. In some embodiments, an individual can be at least about any of 18, 20, 25, 30, 35, 40, 45, 50, 55, 60, 65, 70, 75, 80, or 85 years old. In some embodiments, an individual can be a male. In some embodiments, an individual can be a female. In some embodiments, an individual may have refused surgery. In some embodiments, an individual can be medically inoperable. In some embodiments, an individual can be at a clinical stage of Ta, Tis, T1, T2, T3a, T3b, or T4. In some embodiments, cancer can be recurrent. In some embodiments, an individual can be a human who exhibits one or more symptoms associated with cancer. In some of embodiments, an individual can be genetically or otherwise predisposed (e.g., having a risk factor) to developing cancer.

The methods provided herein can be practiced in an adjuvant setting. In some embodiments, the method is practiced in a neoadjuvant setting, i.e., the method can be carried out before the primary/definitive therapy. In some embodiments, the method is used to treat an individual who has previously been treated. Any of the methods of treatment provided herein can be used to treat an individual who has not previously been treated. In some embodiments, the method is used as a first-line therapy. In some embodiments, the method is used as a second-line therapy.

In some embodiments, there is provided a method of reducing incidence or burden of preexisting cancer tumor metastasis (such as pulmonary metastasis or metastasis to the lymph node) in an individual, comprising administering to the individual an effective amount of a composition disclosed herein. In some embodiments, there is provided a method of prolonging time to disease progression of cancer in an individual, comprising administering to the individual an effective amount of a composition disclosed herein. In some embodiments, there is provided a method of prolonging survival of an individual having cancer, comprising administering to the individual an effective amount of a composition disclosed herein.

In some embodiments, at least one or more chemotherapeutic agents can be administered in addition to the composition disclosed herein. In some embodiments, the one or more chemotherapeutic agents may (but not necessarily) belong to different classes of chemotherapeutic agents.

In some embodiments, there is provided a method of treating a disease (such as cancer) in an individual, comprising administering: (a) a vaccine disclosed herein (e.g., that includes a mutant peptide selected based on a machine-learning technique disclosed herein or a precursor thereof); and (b) an immunomodulator. In some embodiments, there is provided a method of treating a disease (such as cancer) in an individual, comprising administering: (a) a vaccine disclosed herein (e.g., that includes a mutant peptide selected based on a machine-learning technique disclosed herein or a precursor thereof); and (b) an antagonist of a checkpoint protein. In some embodiments, there is provided a method of treating a disease (such as cancer) in an individual, comprising administering: (a) a vaccine disclosed herein (e.g., that includes a mutant peptide selected based on a machine-learning technique disclosed herein or a precursor thereof); and (b) an antagonist of programmed cell death 1 (PD-1), such as anti-PD-1. In some embodiments, there is provided a method of treating a disease (such as cancer) in an individual, comprising administering: (a) a vaccine disclosed herein (e.g., that includes a mutant peptide selected based on a machine-learning technique disclosed herein or a precursor thereof); and (b) an antagonist of programmed death-ligand 1 (PD-L1), such as anti-PD-L1. In some embodiments, there is provided a method of treating a disease (such as cancer) in an individual, comprising administering: (a) a vaccine disclosed herein (e.g., that includes a mutant peptide selected based on a machine-learning technique disclosed herein or a precursor thereof); and (b) an antagonist of cytotoxic T-lymphocyte-associated protein 4 (CTLA-4), such as anti-CTLA-4.

It will be appreciated that various disclosures refer to use of amino acid sequences. Nucleic acid sequences may additionally or alternatively be used. For example, a disease-specific sample can be sequenced to identify a set of nucleic acid sequences that is not present in a corresponding non-disease-specific sample (e.g., from the same subject or a different subject). Similarly, the nucleic acid sequence of an MHC molecule and/or T-cell receptor may further be identified. Representations of each of a nucleic acid disease-specific sequence and of an MHC molecule (or of a T-cell receptor) can be processed by an attention-based model as described herein (e.g., and potentially having been trained using nucleic acid sequence representations).

### Example Model Performance

An example peptide-MHC (MHC Class II) machine-learning model (herein “P-MHC-II Model”) was developed. This model is an example implementation for machine-learning model 132 in FIG. 1. The P-MHC-II Model was implemented in correspondence with the architectures depicted in FIG. 5A. The P-MHC-II Model is compared to other previously available models (e.g., NetMHCpan-4.0 (referred to herein as “Model A”). The P-MHC-II Model performed better than Model A for peptide presentation.

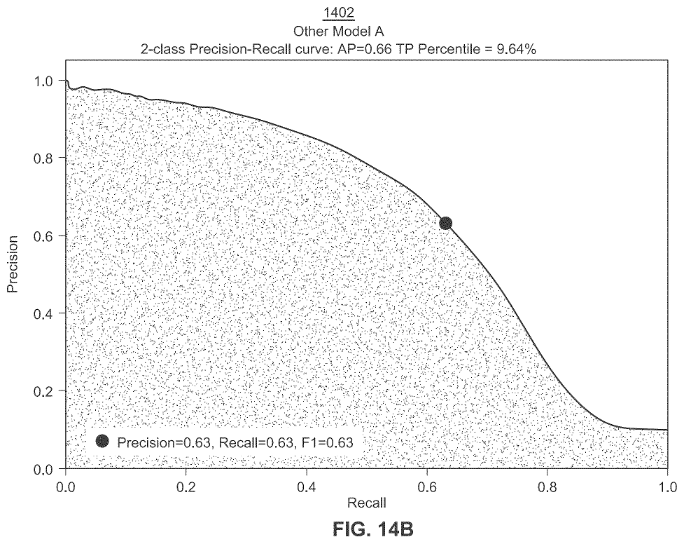

FIGS. 14A and 14B are plots with example precision-recall (PR) curves in accordance with some embodiments. FIGS. 14A and 14B illustrate the performance of the P-MHC-II Model as compared to Model A. An eluted ligand (EL) test dataset was used to evaluate the presentation prediction performance between the EL output of the P-MHC-II Model and the EL output of Model A.

FIG. 14A includes an example plot 1300 indicating the performance of the P-MHC-II Model, in accordance with some embodiments. FIG. 14B includes an example plot 1402 indicating the performance of a previously used approach, Model A, with respect to its elution output, in accordance with some embodiments. The dot on the curve of each of plots 1400 and 1402 corresponds to a score threshold for the top 10.00% and 9.64% quantile, respectively, of the score. Average precision (AP) is representative of threshold-independent performance. The F1 score, precision, and recall values are based on the respective threshold.

Model A values were percentile rank outputs from the previously used approach. The P-MHC-II Model values were taken from the output (of the final node) of the P-MHC-II Model. Based on these PR curves, the results in FIGS. 14A and 14B indicate that P-MHC-II Model showed improved performance over Model A, with an AP value of 0.84 vs 0.66 for Model A. AP values of the methods were compared on a per-allele basis.

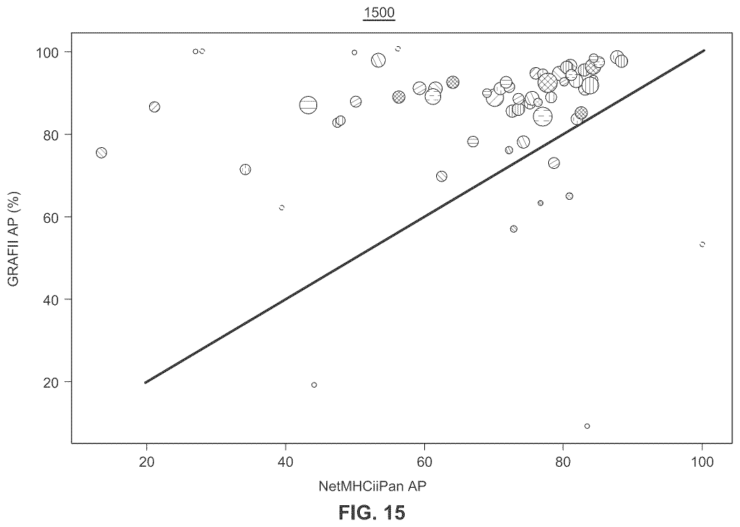

FIG. 15 is an example plot 1500 comparing example average precision values of elution-ligand outputs of Model A and the P-MHC-II Model for each allele in a test data set, in accordance with some embodiments.

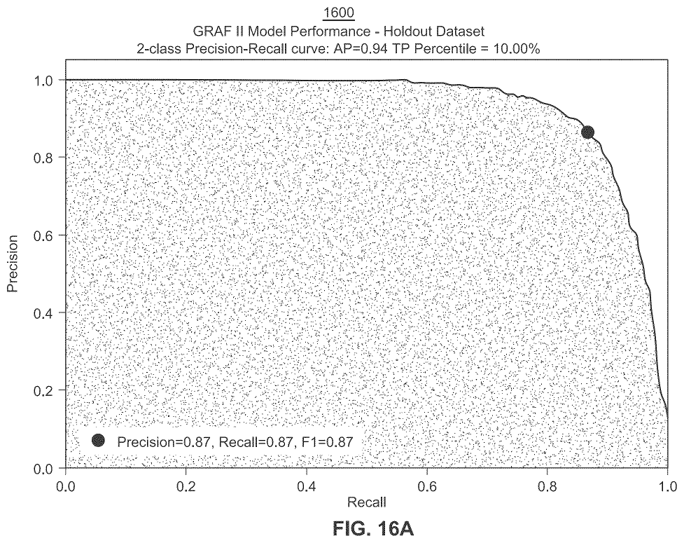

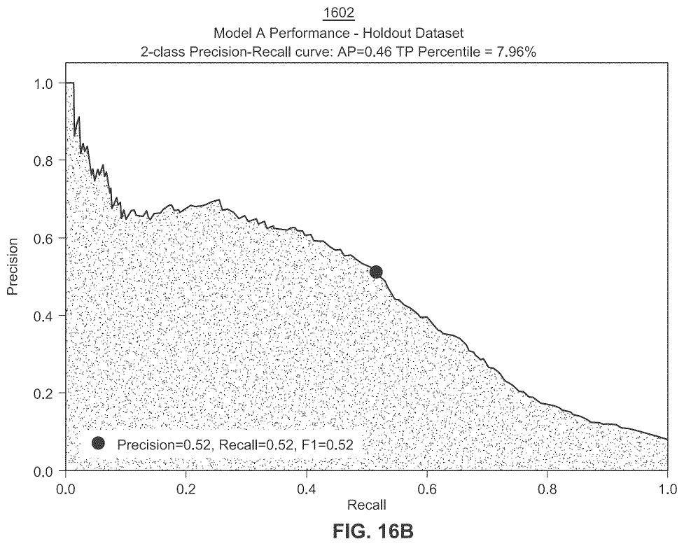

FIGS. 16A-16B are example plots 1600 and 1602 that illustrate the performance of P-MHC-II Model (BA output) and Model A (BA output), respectively, in accordance with some embodiments.

### Example Computer System

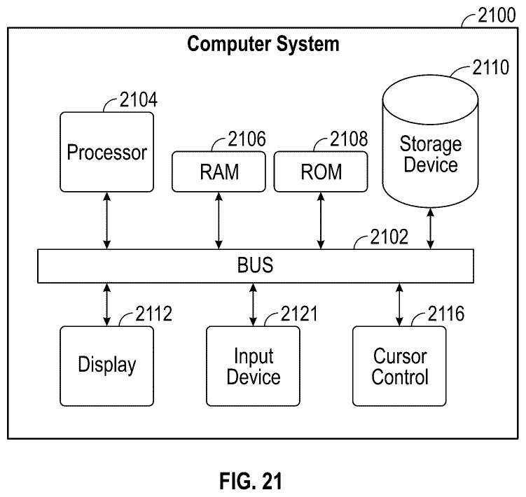

FIG. 21 is a block diagram of a computer system, in accordance with some embodiments. Computer system 2100 can be an example of one implementation for computing platform 102 described above in FIG. 1.

FIG. 21 illustrates an example of one or more computing device(s) 2100 that can be utilized to determined a predicted amino acid-IPC prediction, in accordance with some embodiments. In certain embodiments, the one or more computing device(s) 2100 may perform one or more steps of one or more methods described or illustrated herein. In certain embodiments, the one or more computing device(s) 2100 provide functionality described or illustrated herein. In certain embodiments, software running on the one or more computing device(s) 2100 performs one or more steps of one or more methods described or illustrated herein, or provides functionality described or illustrated herein. Certain embodiments include one or more portions of the one or more computing device(s) 2100.

This disclosure contemplates any suitable number of computing systems 2100. This disclosure contemplates one or more computing device(s) 2100 taking any suitable physical form. As example and not by way of limitation, one or more computing device(s) 2100 can be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the one or more computing device(s) 2100 can be unitary or distributed; span multiple locations; span multiple machines; span multiple data centers; or reside in a cloud, which may include one or more cloud components in one or more networks.

Where appropriate, the one or more computing device(s) 2100 may perform without substantial spatial or temporal limitation one or more steps of one or more methods described or illustrated herein. As an example, and not by way of limitation, the one or more computing device(s) 2100 may perform, in real-time or in batch mode, one or more steps of one or more methods described or illustrated herein. The one or more computing device(s) 2100 may perform, at different times or at different locations, one or more steps of one or more methods described or illustrated herein, where appropriate.

In certain embodiments, the one or more computing device(s) 2100 includes a processor 2102, memory 2104, database 2106, an input/output (I/O) interface 2108, a communication interface 2110, and a bus 2112. Although this disclosure describes and illustrates a particular computer system having a particular number of particular components in a particular arrangement, this disclosure contemplates any suitable computer system having any suitable number of any suitable components in any suitable arrangement. In certain embodiments, processor 2102 includes hardware for executing instructions, such as those making up a computer program. As an example, and not by way of limitation, to execute instructions, processor 2102 may retrieve (or fetch) the instructions from an internal register, an internal cache, memory 2104, or database 2106; decode and execute them; and then write one or more results to an internal register, an internal cache, memory 2104, or database 2106. In certain embodiments, processor 2102 may include one or more internal caches for data, instructions, or addresses. This disclosure contemplates processor 2102 including any suitable number of any suitable internal caches, where appropriate. As an example, and not by way of limitation, processor 2102 may include one or more instruction caches, one or more data caches, and one or more translation lookaside buffers (TLBs). Instructions in the instruction caches can be copies of instructions in memory 2104 or database 2106, and the instruction caches may speed up retrieval of those instructions by processor 2102.

Data in the data caches can be copies of data in memory 2104 or database 2106 for instructions executing at processor 2102 to operate on; the results of previous instructions executed at processor 2102 for access by subsequent instructions executing at processor 2102 or for writing to memory 2104 or database 2106; or other suitable data. The data caches may speed up read or write operations by processor 2102. The TLBs may speed up virtual-address translation for processor 2102. In certain embodiments, processor 2102 may include one or more internal registers for data, instructions, or addresses. This disclosure contemplates processor 2102 including any suitable number of any suitable internal registers, where appropriate. Where appropriate, processor 2102 may include one or more arithmetic logic units (ALUs); be a multi-core processor; or include one or more processors 2102. Although this disclosure describes and illustrates a particular processor, this disclosure contemplates any suitable processor.

In certain embodiments, memory 2104 includes main memory for storing instructions for processor 2102 to execute or data for processor 2102 to operate on. As an example, and not by way of limitation, the one or more computing device(s) 2100 may load instructions from database 2106 or another source (such as, for example, another one or more computing device(s) 2100) to memory 2104. Processor 2102 may then load the instructions from memory 2104 to an internal register or internal cache. To execute the instructions, processor 2102 may retrieve the instructions from the internal register or internal cache and decode them. During or after execution of the instructions, processor 2102 may write one or more results (which can be intermediate or final results) to the internal register or internal cache. Processor 2102 may then write one or more of those results to memory 2104.

In certain embodiments, processor 2102 executes only instructions in one or more internal registers, internal caches, or memory 2104 (as opposed to database 2106 or elsewhere) and operates only on data in one or more internal registers, internal caches, or memory 2104 (as opposed to database 2106 or elsewhere). One or more memory buses (which may each include an address bus and a data bus) may couple processor 2102 to memory 2104. Bus 2112 may include one or more memory buses, as described below. In certain embodiments, one or more memory management units (MMUs) reside between processor 2102 and memory 2104 and facilitate accesses to memory 2104 requested by processor 2102. In certain embodiments, memory 2104 includes random access memory (RAM). This RAM can be volatile memory, where appropriate. Where appropriate, this RAM can be dynamic RAM (DRAM) or static RAM (SRAM). Moreover, where appropriate, this RAM can be single-ported or multi-ported RAM. This disclosure contemplates any suitable RAM. Memory 2104 may include one or more memory devices 2104, where appropriate. Although this disclosure describes and illustrates particular memory, this disclosure contemplates any suitable memory.

In certain embodiments, database 2106 includes mass storage for data or instructions. As an example, and not by way of limitation, database 2106 may include a hard disk drive (HDD), a floppy disk drive, flash memory, an optical disc, a magneto-optical disc, magnetic tape, or a Universal Serial Bus (USB) drive, or a combination of two or more of these. Database 2106 may include removable or non-removable (or fixed) media, where appropriate. Database 2106 can be internal or external to the one or more computing device(s) 2100, where appropriate. In certain embodiments, database 2106 is non-volatile, solid-state memory. In certain embodiments, database 2106 includes read-only memory (ROM). Where appropriate, this ROM can be mask-programmed ROM, programmable ROM (PROM), erasable PROM (EPROM), electrically erasable PROM (EEPROM), electrically alterable ROM (EAROM), flash memory, or a combination of two or more of these. This disclosure contemplates mass database 2106 taking any suitable physical form. Database 2106 may include one or more storage control units facilitating communication between processor 2102 and database 2106, where appropriate. Where appropriate, database 2106 may include one or more databases 2106. Although this disclosure describes and illustrates particular storage, this disclosure contemplates any suitable storage.

In certain embodiments, I/O interface 2108 includes hardware, software, or both, providing one or more interfaces for communication between the one or more computing device(s) 2100 and one or more I/O devices. The one or more computing device(s) 2100 may include one or more of these I/O devices, where appropriate. One or more of these I/O devices may enable communication between a person and the one or more computing device(s) 2100. As an example, and not by way of limitation, an I/O device may include a keyboard, keypad, microphone, monitor, mouse, printer, scanner, speaker, still camera, stylus, tablet, touch screen, trackball, video camera, another suitable I/O device, or a combination of two or more of these. An I/O device may include one or more sensors. This disclosure contemplates any suitable I/O devices and any suitable I/O interfaces 2108 for them. Where appropriate, I/O interface 2108 may include one or more device or software drivers enabling processor 2102 to drive one or more of these I/O devices. I/O interface 2108 may include one or more I/O interfaces 2108, where appropriate. Although this disclosure describes and illustrates a particular I/O interface, this disclosure contemplates any suitable I/O interface.

In certain embodiments, communication interface 2110 includes hardware, software, or both providing one or more interfaces for communication (such as, for example, packet-based communication) between the one or more computing device(s) 2100 and one or more other computing device(s) 2100 or one or more networks. As an example, and not by way of limitation, communication interface 2110 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 2110 for it.

As an example, and not by way of limitation, the one or more computing device(s) 2100 may communicate with an ad hoc network, a personal area network (PAN), a local area network (LAN), a wide area network (WAN), a metropolitan area network (MAN), one or more portions of the Internet, or a combination of two or more of these. One or more portions of one or more of these networks can be wired or wireless. As an example, the one or more computing device(s) 2100 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), other suitable wireless network, or a combination of two or more of these. The one or more computing device(s) 2100 may include any suitable communication interface 2110 for any of these networks, where appropriate. Communication interface 2110 may include one or more communication interfaces, where appropriate. Although this disclosure describes and illustrates a particular communication interface, this disclosure contemplates any suitable communication interface.

In certain embodiments, bus 2112 includes hardware, software, or both coupling components of the one or more computing device(s) 2100 to each other. As an example, and not by way of limitation, bus 2112 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, another suitable bus, or a combination of two or more of these. Bus 2112 may include one or more buses 2112, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium can be volatile, non-volatile, or a combination of volatile and non-volatile, where appropriate.

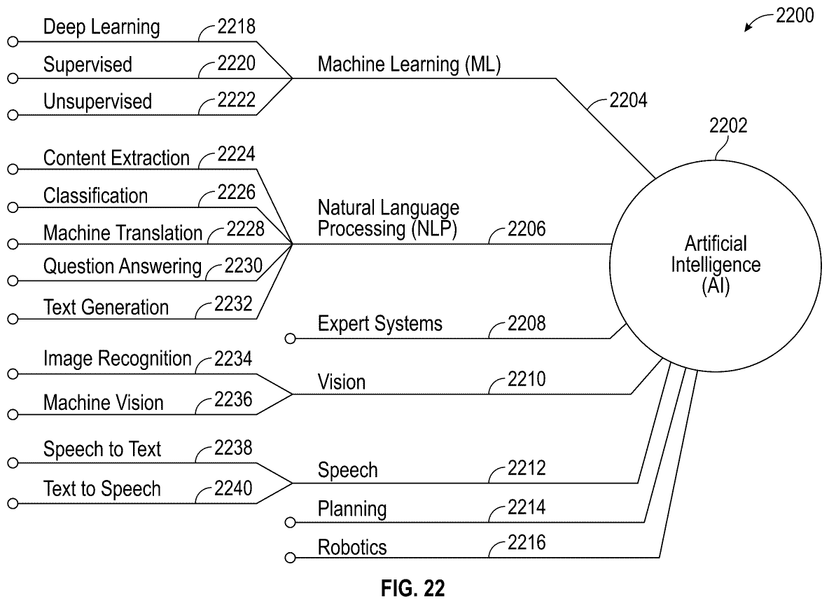

FIG. 22 illustrates a diagram 2200 of an example artificial intelligence (AI) architecture 2202 (which can be included as part of the one or more computing device(s) 2100 as discussed above with respect to FIG. 21) that can be utilized to determined one or more predicted amino acid-IPC interactions, in accordance with the disclosed embodiments. In certain embodiments, the AI architecture 2202 can be implemented utilizing, for example, one or more processing devices that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), and/or other processing device(s) that can be suitable for processing various molecular data and making one or more decisions based thereon), software (e.g., instructions running/executing on one or more processing devices), firmware (e.g., microcode), or some combination thereof.

In certain embodiments, as depicted by FIG. 22, the AI architecture 2202 may include machine learning (ML) algorithms and functions 2204, natural language processing (NLP) algorithms and functions 2206, expert systems 2208, computer-based vision algorithms and functions 2210, speech recognition algorithms and functions 2212, planning algorithms and functions 2214, and robotics algorithms and functions 2216. In certain embodiments, the ML algorithms and functions 2204 may include any statistics-based algorithms that can be suitable for finding patterns across large amounts of data (e.g., “Big Data” such as genomics data, proteomics data, metabolomics data, metagenomics data, transcriptomics data, or other omics data). For example, in certain embodiments, the ML algorithms and functions 2204 may include deep learning algorithms 2218, supervised learning algorithms 2220, and unsupervised learning algorithms 2222.

In certain embodiments, the deep learning algorithms 2218 may include any artificial neural networks (ANNs) that can be utilized to learn deep levels of representations and abstractions from large amounts of data. For example, the deep learning algorithms 2218 may include ANNs, such as a perceptron, a multilayer perceptron (MLP), an autoencoder (AE), a convolution neural network (CNN), a recurrent neural network (RNN), long short term memory (LSTM), a grated recurrent unit (GRU), a restricted Boltzmann Machine (RBM), a deep belief network (DBN), a bidirectional recurrent deep neural network (BRDNN), a generative adversarial network (GAN), and deep Q-networks, a neural autoregressive distribution estimation (NADE), an adversarial network (AN), attentional models (AM), a spiking neural network (SNN), deep reinforcement learning, and so forth.

In certain embodiments, the supervised learning algorithms 2220 may include any algorithms that can be utilized to apply, for example, what has been learned in the past to new data using labeled examples for predicting future events. For example, starting from the analysis of a known training data set, the supervised learning algorithms 2220 may produce an inferred function to make predictions about the output values. The supervised learning algorithms 2220 may also compare its output with the correct and intended output and find errors in order to modify the supervised learning algorithms 2220 accordingly. On the other hand, the unsupervised learning algorithms 2222 may include any algorithms that may applied, for example, when the data used to train the unsupervised learning algorithms 2222 are neither classified nor labeled. For example, the unsupervised learning algorithms 2222 may study and analyze how systems may infer a function to describe a hidden structure from unlabeled data.

In certain embodiments, the NLP algorithms and functions 2206 may include any algorithms or functions that can be suitable for automatically manipulating natural language, such as speech and/or text. For example, the NLP algorithms and functions 2206 may include content extraction algorithms or functions 2224, classification algorithms or functions 2226, machine translation algorithms or functions 2228, question answering (QA) algorithms or functions 2230, and text generation algorithms or functions 2232. In certain embodiments, the content extraction algorithms or functions 2224 may include a means for extracting text or images from electronic documents (e.g., webpages, text editor documents, and so forth) to be utilized, for example, in other applications.

In certain embodiments, the classification algorithms or functions 2226 may include any algorithms that may utilize a supervised learning model (e.g., logistic regression, naïve Bayes, stochastic gradient descent (SGD), k-nearest neighbors, decision trees, random forests, support vector machine (SVM), and so forth) to learn from the data input to the supervised learning model and to make new observations or classifications based thereon. The machine translation algorithms or functions 2228 may include any algorithms or functions that can be suitable for automatically converting source text in one language, for example, into text in another language. The QA algorithms or functions 2230 may include any algorithms or functions that can be suitable for automatically answering questions posed by humans in, for example, a natural language, such as that performed by voice-controlled personal assistant devices. The text generation algorithms or functions 2232 may include any algorithms or functions that can be suitable for automatically generating natural language texts.

In certain embodiments, the expert systems 2208 may include any algorithms or functions that can be suitable for simulating the judgment and behavior of a human or an organization that has expert knowledge and experience in a particular field (e.g., stock trading, medicine, sports statistics, and so forth). The computer-based vision algorithms and functions 2210 may include any algorithms or functions that can be suitable for automatically extracting information from images (e.g., photo images, video images). For example, the computer-based vision algorithms and functions 2210 may include image recognition algorithms 2234 and machine vision algorithms 2236. The image recognition algorithms 2234 may include any algorithms that can be suitable for automatically identifying and/or classifying objects, places, people, and so forth that can be included in, for example, one or more image frames or other displayed data. The machine vision algorithms 2236 may include any algorithms that can be suitable for allowing computers to “see”, or, for example, to rely on image sensors cameras with specialized optics to acquire images for processing, analyzing, and/or measuring various data characteristics for decision making purposes.

In certain embodiments, the speech recognition algorithms and functions 2212 may include any algorithms or functions that can be suitable for recognizing and translating spoken language into text, such as through automatic speech recognition (ASR), computer speech recognition, speech-to-text (STT) 2238, or text-to-speech (TTS) 2240 in order for the computing to communicate via speech with one or more users, for example. In certain embodiments, the planning algorithms and functions 2214 may include any algorithms or functions that can be suitable for generating a sequence of actions, in which each action may include its own set of preconditions to be satisfied before performing the action. Examples of AI planning may include classical planning, reduction to other problems, temporal planning, probabilistic planning, preference-based planning, conditional planning, and so forth. Lastly, the robotics algorithms and functions 2216 may include any algorithms, functions, or systems that may enable one or more devices to replicate human behavior through, for example, motions, gestures, performance tasks, decision-making, emotions, and so forth.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Herein, “automatically” and its derivatives means “without human intervention,” unless expressly indicated otherwise or indicated otherwise by context.

The embodiments disclosed herein are only examples, and the scope of this disclosure is not limited to them. Embodiments according to this disclosure are in particular disclosed in the attached claims directed to a method, a storage medium, a system and a computer program product, wherein any feature mentioned in one claim category, e.g. method, can be claimed in another claim category, e.g. system, as well. The dependencies or references back in the attached claims are chosen for formal reasons only. However, any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates certain embodiments as providing particular advantages, certain embodiments may provide none, some, or all of these advantages.

### Example Descriptions of Terms

As used herein, the terms “peptide,” “polypeptide,” and “protein” are used interchangeably to refer to a polymer of amino acid residues. The terms encompass amino acid chains of any length, including full-length proteins with amino acid residues linked by covalent peptide bonds.

As used herein, a “mutant peptide” may refer to a peptide that is not present in the normal tissue (e.g., in the wild type amino acid sequences of normal tissue) of an individual subject. A mutant peptide comprises at least one mutant amino acid and can be present in a diseased tissue (e.g., collected from a particular subject), but not in a normal tissue (e.g., collected from the particular subject, collected from a different subject, and/or as identified in a database as corresponding to normal tissue). A mutant peptide may include an epitope. An epitope is the portion of a mutant peptide to which an MHC molecule or a TCR binds. Thus, this binding between the epitope of the mutant peptide and the MHC molecule or TCR can induce an immune response (as a result of the mutant peptide not being associated with a subject's “self”). A mutant peptide can include or be a neoantigen. A mutant peptide can arise from, as non-limiting examples: a non-synonymous mutation leading to different amino acids in the protein (e.g., point mutation); a read-through mutation in which a stop codon is modified or deleted, leading to translation of a longer protein with a novel tumor-specific sequence at the C-terminus; a splice site mutation that leads to a unique tumor-specific protein sequence; a chromosomal rearrangement that gives rise to a chimeric protein with a tumor-specific sequence at a junction of two proteins (gene fusion); and/or a frameshift insertion or deletion that leads to a new open reading frame with a tumor-specific protein sequence. A mutant peptide can include a polypeptide (as characterized by a polypeptide sequence) and/or can be encoded by a nucleotide sequence.

As used herein, a “C-flank” of a peptide refers to one or more amino acids upstream of the C-terminus of the peptide, from the parent protein. Optionally, a C-flank of a peptide includes one, two, three, four, five, or more amino acid residues upstream of the C-terminus of the peptide.

As used herein, an “N-flank” of a peptide refers to one or more amino acids downstream of the N-terminus of the peptide, from the parent protein. Optionally, an N-flank of a peptide includes one, two, three, four, five, or more amino acid residues downstream of the N-terminus of the peptide.

As used herein, an “epitope” of a peptide may refer to a region of the peptide between the C-flank and N-flank and can be recognized by a TCR. The epitope of the peptide is a part of the peptide that is recognized by a TCR on a T cell and MHC I on an antigen-presenting cell. For example, the epitope can be a peptide to which a TCR binds, such as a peptide to which the TCR binds when the peptide is bound to MHC I on an antigen-presenting cell.

As used herein, a “ligand” is a peptide that is found to be presented by an MHC molecule at the cell surface from elution experiments or found to be bound to MHC in an in vitro assay.

As used herein, a “sequence” refers to an amino acid sequence that includes an ordered set of amino acid identifiers.

As used herein, a “peptide sequence” refers to a sequence that identifies amino acids of at least a portion of a peptide. In some cases, the peptide sequence includes a variant-coding sequence that includes a variant that is not observed in a corresponding reference sequence.

When the peptide includes a mutant peptide, the variant-coding sequence, identifies amino acids of the mutation or variant. However, when the peptide does not include a mutation or variant, the variant-coding sequence does not identify amino acids of a mutation or variant (and in that instance, is the same as the reference sequence). A variant-coding sequence can be determined by collecting a disease and/or tumor sample (e.g., that includes tumor cells) and performing a sequencing analysis to identify one or more sequences corresponding to disease and/or tumor cells in the sample. In some instances, a sequencing analysis outputs an amino acid sequence. In some instances, a sequencing analysis outputs a nucleic acid sequence, which can be subsequently processed to transform codons into amino acid identifiers and thus to produce an amino acid sequence. A variant-coding sequence can include a sequence of a neoantigen. A variant-coding sequence may, but need not, include one or more termini (e.g., the C-terminus and/or the N-terminus) of the peptide. A variant-coding sequence may include an epitope of the peptide. A variant-coding sequence can identify amino acids within a peptide having one or more variants (e.g., one or more amino acid distinctions) relative to a corresponding reference sequence. In some instances, a variant-coding sequence includes an ordered set of amino acids. In some instances, a variant-coding sequence identifies a reference peptide (e.g., by identifying a genetic reference sequence, such as by gene, start position, and/or end position; or by gene, start position, and/or length) and one or more point mutations relative to the reference peptide.

As used herein, a “reference sequence” may refer to a sequence that identifies amino acids within at least part of a non-mutant peptide or wild-type peptide (e.g., wild-type, parental sequence). The non-mutant or wild-type peptide may include no variants or fewer variants than are included in a mutant peptide. The reference sequence may include an amino acid sequence encoded by a genetic sequence within a same gene relative to a gene that includes a corresponding variant-coding sequence. The reference sequence may include an amino acid sequence encoded by a genetic sequence spanning the same start and stop within a gene relative to intra-gene positions associated with a genetic sequence associated with a corresponding variant-coding sequence. The reference sequence can be identified by collecting a non-disease and/or non-tumor sample from one or more subjects (who may, but need not, include a subject from which a disease sample was collected to determine a variant-coding sequence) and performing a sequencing analysis using the sample.

As used herein, a “pseudosequence” of an MHC molecule may refer to an ordered set of amino acids of the MHC molecule that typically contacts a peptide.

As used herein, a “representation” of a sequence or “sequence representation” can include a set of values that represent or identify amino acids in the sequence and/or a set of values that represent or identify nucleic acids that encode the sequence. For example, each amino acid can be represented by a binary string and/or vector of values that is distinct from each other binary string and/or vector representing each other amino acid. The sequence representation can be generated using, for example, one-hot encoding or using a BLOcks SUbstitution Matrix (BLOSUM) matrix. For example, a multi-dimensional (e.g., 20- or 21-dimensional) array be initialized (e.g., randomly or pseudo-randomly initialized). The initialized array may include, for each amino acid, a unique vector corresponding to that amino acid. The values can be fixed such that use of such a unique vector can be assumed to represent the corresponding amino acid. There can be multiple possible nucleic acid representations of a given sequence, given that any of multiple codons can encode a single amino acid.

As used herein, “presentation” of a peptide refers to at least part of the peptide being presented on a surface of a cell by virtue of being bound to an MHC molecule in a particular manner. The presented peptide can then be accessible to other cells, such as nearby T cells.

As used herein, a “sample” can include tissue (e.g., a biopsy), single cell, multiple cells, fragments of cells, or an aliquot of body fluid. The sample can be obtained from a subject by means such as, for example, without limitation, venipuncture, excretion, ejaculation, massage, biopsy, needle aspirate, lavage sample, scraping, surgical incision, intervention, another type of sample collection means, or a combination thereof.

As used herein, a “subject” encompasses one or more cells, tissue, or an organism. The subject can be a human or non-human, whether in vivo, ex vivo, or in vitro, male or female. A subject can be a mammal, such as a human.

As used herein, “binding affinity” refers to affinity of binding between an amino acid (e.g., a peptide of a specific antigen) and an IPC (e.g., an MHC molecule and/or MHC allele). The binding affinity may characterize a stability, tendency, and/or strength of the binding between the peptide and an IPC.

As used herein, “immunogenicity” may refer to the ability to elicit an immune response (e.g., via T cells and/or B cells). A peptide that is “immunogenic” can be one that is capable of eliciting an immune response.

As used herein, “MHC” refers to the major histocompatibility complex. The human MHC is also called the human leukocyte antigen (HLA) complex.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed can be resorted to by those skilled in the art, and that such modifications and variations are considered to be within the scope of this invention as defined by the appended claims.

### Example Embodiments

Embodiments disclosed herein may include:

1. A computer-implemented method for predicting an amino acid-immunoprotein complex (IPC) interaction comprising:
accessing a set of amino acid sequences, each of the amino acid sequences of the set having been identified from at least one protein;
accessing an immunoprotein complex (IPC) sequence identified for an IPC of a subject;
processing, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
processing, using a second processing block in the processing subsystem, an IPC sequence representation to generate a transformed IPC sequence representation, wherein the IPC sequence representation was generated based on the identified IPC sequence appended with a BOS token, and wherein the set of amino acid sequence representations and the IPC sequence representation are processed in parallel;
generating composite representations by combining each of the transformed BOS token representations of the set of transformed amino acid sequence representations with the transformed BOS token representation of the transformed IPC sequence representation; and
determining one or more predicted amino acid-IPC interactions based on the composite representations.

2. The computer-implemented method of embodiment 1, wherein the set of transformed amino acid sequence representations comprises a set of amino-terminal flanking (N-flank) representations or a set of carboxy-terminal flanking (C-flank) representations.

3. The computer-implemented method of embodiment 1, wherein the IPC of the subject is a major histocompatibility complex (MHC).

4. The computer-implemented method of embodiment 3, wherein the set of transformed amino acid sequence representations comprises a set of MHC-binding representations.

5. The computer-implemented method of embodiment 3, wherein the MHC comprises MHC class II (MHC-II).

6. The computer-implemented method of embodiment 3, wherein the MHC comprises MHC class I (MHC-I).

7. The computer-implemented method of embodiment 1, wherein the IPC of the subject is a T-cell receptor (TCR).

8. The computer-implemented method of embodiment 1, wherein the at least one protein is a therapeutic protein.

9. The computer-implemented method of embodiment 1, wherein the at least one protein is present in a disease sample from the subject.

10. The computer-implemented method of embodiment 9, wherein the disease sample is a tumor cell biopsy.

11. The computer-implemented method of embodiment 9, wherein the disease sample includes cancer.

12. The computer-implemented method of embodiment 9, wherein the disease sample includes tissue.

13. The computer-implemented method of embodiment 1, wherein generating composite representations comprises:
for each of the set of transformed amino acid sequence representations, elementwise multiplying a transformed amino acid beginning-of-sequence (BOS) representation corresponding to the transformed amino acid sequence representation by a transformed IPC beginning-of-sequence (BOS) representation corresponding to the transformed IPC sequence representation.

14. The computer-implemented method of embodiment 1, wherein processing a set of amino acid sequence representations comprises processing a peptide beginning-of-sequence (BOS) representation to generate a transformed peptide sequence representation.

15. The computer-implemented method of embodiment 1, wherein processing an IPC sequence representation comprises processing an MHC beginning-of-sequence representation (BOS) to generate a transformed MHC sequence representation.

16. The computer-implemented method of embodiment 1, further comprising:
for each of a set of IPC sequences, performing the steps of: accessing the IPC sequence, processing the set of amino acid sequence representations, processing the IPC sequence representation using the respective IPC sequence, and generating the composite representations;
wherein the determined one or more predicted amino acid-IPC interactions are based on the composite representations corresponding to the set of IPC sequences.

17. The computer-implemented method of embodiment 16, wherein the set of IPC sequences comprises twelve major histocompatibility complex (MHC) allotypes of the subject and/or six MHC alleles of the subject.

18. The computer-implemented method of embodiment 16, further comprising:
selecting one or more peptide-IPC combinations from the set of amino acid sequences and the set of IPC sequences to include as a target for an immunotherapy based on the one or more determined amino-acid IPC interactions.

19. The computer-implemented method of embodiment 1, wherein processing the set of amino acid sequence representations comprises:
transforming the set of amino acid sequence representations using the one or more first processing blocks into the set of transformed amino acid sequence representations, wherein each of the one or more first processing blocks includes a set of processing sub-blocks.

20. The computer-implemented method of embodiment 1, further comprising:
embedding the set of amino acid sequences to generate a set of embedded amino acid sequence representations; and
positionally encoding the set of embedded amino acid sequence representations.

21. The computer-implemented method of embodiment 1, wherein processing the IPC sequence representation comprises:
transforming the IPC sequence representation using the second processing block into a transformed IPC sequence representation, wherein the second processing block includes a set of processing sub-blocks.

22. The computer-implemented method of embodiment 1, further comprising:
embedding the IPC sequence to generate an embedded IPC sequence representation; and
positionally encoding the embedded IPC sequence representation.

23. The computer-implemented method of embodiment 1, wherein the machine-learning model includes one or more transformer encoders, each of the one or more transformer encoders including a processing layer.

24. The computer-implemented method of embodiment 1, wherein:
each of the one or more first processing blocks or the second processing block comprises a set of processing sub-blocks; and
each of the set of processing sub-blocks includes a neural network comprising at least one processing layer.

25. The computer-implemented method of embodiment 1, wherein the set of amino acid sequence representations comprises aggregate sequence representations, the aggregate sequence representations including a set of peptide representations and one or more of: a set of amino-terminal flanking (N-flank) representations or a set of carboxy-terminal flanking (C-flank) representations.

26. The computer-implemented method of embodiment 1, further comprising, prior to generating the set of transformed amino acid sequence representations:
flattening the aggregate sequence representations into a single array; and
densifying the aggregate sequence representations by removing empty rows from the array, wherein the transformed amino acid sequence representations are generated based on the densified aggregate sequence representations.

27. The computer-implemented method of embodiment 1, wherein the IPC sequence representation comprises aggregate sequence representations, the aggregate sequence representations including one or more of: a major histocompatibility complex (MHC) sequence representation or a T-cell receptor (TCR) sequence representation.

28. The computer-implemented method of embodiment 1, wherein processing the set of amino acid sequence representations comprises:
for each amino acid sequence representation of the set:
   determining, for each element of the amino acid sequence representation, a plurality of vectors based on a set of weights associated with a processing layer of the machine-learning model; and
   generating the set of element-focused scores based on the plurality of vectors and the set of weights.

29. The computer-implemented method of embodiment 28, wherein the plurality of vectors comprises a key vector, a value vector, and a query vector, and the set of weights comprises a set of key weights, a set of value weights, and a set of query weights.

30. The computer-implemented method of embodiment 28, wherein generating the set of element-focused scores comprises:
determining each element-focused score from each pair of elements from the query vector and the key vector.

31. The computer-implemented method of embodiment 1, wherein the one or more first processing blocks and the second processing block comprise attention blocks, each attention block comprising a set of attention sub-blocks, each attention sub-block comprising a self-attention layer; and
wherein the machine-learning model is an attention-based machine learning model.

32. The computer-implemented method of embodiment 31, further comprising:
by one or more of the attention blocks, generating attention maps including one or more masks limiting attention applied by the attention sub-blocks to a sequence length according to the masks.

33. The computer-implemented method of embodiment 1, further comprising:
processing, using a fully connected block in an output subsystem of the machine-learning model, the composition representations to generate a first output;
applying, using a dropout block in the output subsystem of the machine-learning model, a dropout to the first output to generate a second output; and
selecting, using a max layer in the output subsystem of the machine-learning model, a subset of the second output to generate a result,
wherein the one or more predicted amino acid-IPC interactions are determined based on the result.

34. The computer-implemented method of embodiment 1, wherein the set of amino acid sequences comprises a peptide sequence and the IPC sequence comprises a major histocompatibility complex (MHC) sequence, and
wherein the one or more predicted amino acid-IPC interactions comprise one or more of:
an interaction affinity prediction for a peptide-IPC combination that predicts a binding affinity between a peptide and MHC;
an interaction prediction for the peptide-IPC combination that predicts whether the MHC will present the peptide at a cell surface; or
an immunogenicity prediction for the peptide-IPC combination that predicts an ability of the peptide to provoke an immune response with respect to the MHC.

35. The computer-implemented method of embodiment 1, wherein determining the one or more predicted amino acid-IPC interactions comprises:
processing the composite representations to generate a set of results; and
selecting an amino acid-IPC combination based on a highest result among the set of results.

36. The computer-implemented method of embodiment 1, wherein the one or more predicted amino acid-IPC interactions comprise a prediction of tumor-specific immunogenicity of a peptide.

37. The computer-implemented method of embodiment 1, wherein the set of amino acid sequences comprises a set of peptide sequences, wherein the one or more predicted amino acid-IPC interactions identify a subset of peptide sequences having increased tumor-specific immunogenicity or increased likelihood of presentation by the IPC relative to the set of peptide sequences.

38. The computer-implemented method of embodiment 1, further comprising:
identifying a subset of peptides from the set of amino acid sequences to include in an individualized vaccine based on the determined one or more predicted amino acid-IPC interactions.

39. The computer-implemented method of embodiment 38, further comprising:
generating a treatment recommendation that includes the individualized vaccine.

40. The computer-implemented method of embodiment 1, further comprising:
selecting a subset of peptides from the set of amino acid sequences to include as a target for an immunotherapy based on the determined one or more predicted amino acid-IPC interactions.

41. The computer-implemented method of embodiment 40, wherein the immunotherapy comprises one or more of: a T cell therapy, a personalized cancer therapy, an antigen-specific immunotherapy, an antigen-dependent immunotherapy, a vaccine, or a natural killer (NK) cell therapy.

42. The computer-implemented method of embodiment 1, further comprising:
selecting a subset of peptides from the set of amino acid sequences to exclude as a target for an immunotherapy based on the determined one or more predicted amino acid-IPC interactions.

43. The computer-implemented method of embodiment 42, wherein the immunotherapy comprises one or more of: a T cell therapy, a personalized cancer therapy, an antigen-specific immunotherapy, an antigen-dependent immunotherapy, a vaccine, or a natural killer (NK) cell therapy.

44. A system for selecting one or more peptides among a set of peptides for inclusion in a pharmaceutical composition, comprising:
one or more non-transitory computer-readable storage media including instructions; and
one or more processors coupled to the one or more storage media, the one or more processors configured to execute the instructions to:
   access a set of amino acid sequences, each of the amino acid sequences of the set having been identified from at least one protein;
   access an immunoprotein complex (IPC) sequence identified for an IPC of a subject;
   process, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
   process, using a second processing block in the processing subsystem, an IPC sequence representation to generate a transformed IPC sequence representation, wherein the set of amino acid sequence representations and the IPC sequence representation are processed in parallel, wherein the IPC sequence representation was generated based on the identified IPC sequence appended with a BOS token;
   generate composite representations by combining each of the transformed BOS token representations of the set of transformed amino acid sequence representations with the transformed BOS token representations of the transformed IPC sequence representation;
   determine one or more predicted amino acid-IPC interactions based on the composite representations; and
   select one or more amino acid-IPC combinations based on the one or more predicted amino-acid IPC interactions,
   wherein the selected one or more peptides correspond to the selected one or more amino acid-IPC combinations.

45. The system of embodiment 44, wherein the set of transformed amino acid sequence representations comprises a set of amino-terminal flanking (N-flank) representations or a set of carboxy-terminal flanking (C-flank) representations.

46. The system of embodiment 44, wherein the IPC of the subject is a major histocompatibility complex (MHC).

47. The system of embodiment 46, wherein the set of transformed amino acid sequence representations comprises a set of MHC-binding representations.

48. The system of embodiment 46, wherein the MHC comprises MHC class II (MHC-II).

49. The system of embodiment 46, wherein the MHC comprises MHC class I (MHC-I).

50. The system of embodiment 44, wherein the IPC of the subject is a T-cell receptor (TCR).

51. The system of embodiment 44, wherein the at least one protein is a therapeutic protein.

52. The system of embodiment 44, wherein the at least one protein is present in a disease sample from the subject.

53. The system of embodiment 52, wherein the disease sample is a tumor cell biopsy.

54. The system of embodiment 52, wherein the disease sample includes cancer.

55. The system of embodiment 52, wherein the disease sample includes tissue.

56. The system of embodiment 44, wherein generating composite representations comprises:
for each of the set of transformed amino acid sequence representations, elementwise multiplying a transformed amino acid beginning-of-sequence (BOS) representation corresponding to the transformed amino acid sequence representation by a transformed IPC beginning-of-sequence (BOS) representation corresponding to the transformed IPC sequence representation.

57. The system of embodiment 44, wherein processing a set of amino acid sequence representations comprises processing a peptide beginning-of-sequence (BOS) representation to generate a transformed peptide sequence representation.

58. The system of embodiment 44, wherein processing an IPC sequence representation comprises processing an MHC beginning-of-sequence representation (BOS) to generate a transformed MHC sequence representation.

59. The system of embodiment 44, wherein the one or more processors are further configured to execute the instructions to:
for each of a set of IPC sequences, performing the steps of: accessing the IPC sequence, processing the set of amino acid sequence representations, processing the IPC sequence representation using the respective IPC sequence, and generating the composite representations;
wherein the determined one or more predicted amino acid-IPC interactions are based on the composite representations corresponding to the set of IPC sequences.

60. The system of embodiment 59, wherein the set of IPC sequences comprises twelve major histocompatibility complex (MHC) allotypes of the subject and/or six MHC alleles of the subject.

61. The system of embodiment 59, wherein the one or more processors are further configured to execute the instructions to:
selecting one or more peptide-IPC combinations from the set of amino acid sequences and the set of IPC sequences to include as a target for an immunotherapy based on the one or more determined amino-acid IPC interactions.

62. The system of embodiment 44, wherein processing the set of amino acid sequence representations comprises:
transforming the set of amino acid sequence representations using the one or more first processing blocks into the set of transformed amino acid sequence representations, wherein each of the one or more first processing blocks includes a set of processing sub-blocks.

63. The system of embodiment 44, wherein the one or more processors are further configured to execute the instructions to:
embedding the set of amino acid sequences to generate a set of embedded amino acid sequence representations; and
positionally encoding the set of embedded amino acid sequence representations.

64. The system of embodiment 44, wherein processing the IPC sequence representation comprises:
transforming the IPC sequence representation using the second processing block into a transformed IPC sequence representation, wherein the second processing block includes a set of processing sub-blocks.

65. The system of embodiment 44, wherein the one or more processors are further configured to execute the instructions to:
embedding the IPC sequence to generate an embedded IPC sequence representation; and
positionally encoding the embedded IPC sequence representation.

66. The system of embodiment 44, wherein the machine-learning model includes one or more transformer encoders, each of the one or more transformer encoders including a processing layer.

67. The system of embodiment 44, wherein:
each of the one or more first processing blocks or the second processing block comprises a set of processing sub-blocks; and
each of the set of processing sub-blocks includes a neural network comprising at least one processing layer.

68. The system of embodiment 44, wherein the set of amino acid sequence representations comprises aggregate sequence representations, the aggregate sequence representations including a set of peptide representations and one or more of: a set of amino-terminal flanking (N-flank) representations or a set of carboxy-terminal flanking (C-flank) representations.

69. The system of embodiment 44, wherein the one or more processors configured to execute the instructions to: prior to generating the set of transformed amino acid sequence representations:
flattening the aggregate sequence representations into a single array; and
densifying the aggregate sequence representations by removing empty rows from the array, wherein the transformed amino acid sequence representations are generated based on the densified aggregate sequence representations.

70. The system of embodiment 44, wherein the IPC sequence representation comprises aggregate sequence representations, the aggregate sequence representations including one or more of: a major histocompatibility complex (MHC) sequence representation or a T-cell receptor (TCR) sequence representation.

71. The system of embodiment 44, wherein processing the set of amino acid sequence representations comprises:
for each amino acid sequence representation of the set:
   determining, for each element of the IPC sequence representation, a plurality of vectors based on a set of weights associated with a processing layer of the machine-learning model; and
   generating the set of element-focused scores based on the plurality of vectors and the set of weights.

72. The system of embodiment 71, wherein the plurality of vectors comprises a key vector, a value vector, and a query vector, and the set of weights comprises a set of key weights, a set of value weights, and a set of query weights.

73. The system of embodiment 71, wherein generating the set of element-focused scores comprises:
determining each element-focused score from each pair of elements from the query vector and the key vector.

74. The system of embodiment 44, wherein the one or more first processing blocks and the second processing block comprise attention blocks, each attention block comprising a set of attention sub-blocks, each attention sub-block comprising a self-attention layer; and wherein the machine-learning model is an attention-based machine learning model.

75. The system of embodiment 74, wherein the one or more processors are further configured to execute the instructions to:
by one or more of the attention blocks, generating attention maps including masks limiting attention applied by the attention sub-blocks to a sequence length according to the masks.

76. The system of embodiment 44, wherein the one or more processors are further configured to execute the instructions to:
processing, using a fully connected block in an output subsystem of the machine-learning model, the composition representations to generate a first output;
applying, using a dropout block in the output subsystem of the machine-learning model, a dropout to the first output to generate a second output; and
selecting, using a max layer in the output subsystem of the machine-learning model, a subset of the second output to generate a result,
wherein the one or more predicted amino acid-IPC interactions are determined based on the result.

77. The system of embodiment 44, wherein the set of amino acid sequences comprises a peptide sequence and the IPC sequence comprises a major histocompatibility complex (MHC) sequence, and
wherein the one or more predicted amino acid-IPC interactions comprise one or more of:
an interaction affinity prediction for a peptide-IPC combination that predicts a binding affinity between a peptide and MHC;
an interaction prediction for the peptide-IPC combination that predicts whether the MHC will present the peptide at a cell surface; or
an immunogenicity prediction for the peptide-IPC combination that predicts an ability of the peptide to provoke an immune response with respect to the MHC.

78. The system of embodiment 44, wherein determining the one or more predicted amino acid-IPC interactions comprises:
processing the composite representations to generate a set of results; and
selecting an amino acid-IPC combination based on a highest result among the set of results.

79. The system of embodiment 44, wherein the one or more predicted amino acid-IPC interactions comprise a prediction of tumor-specific immunogenicity of a peptide.

80. The system of embodiment 44, wherein the set of amino acid sequences comprises a set of peptide sequences, wherein the one or more predicted amino acid-IPC interactions identify a subset of peptide sequences having increased tumor-specific immunogenicity or increased likelihood of presentation by the IPC relative to the set of peptide sequences.

81. The system of embodiment 44, wherein the one or more processors are further configured to execute the instructions to:
identifying a subset of peptides from the set of amino acid sequences to include in an individualized vaccine based on the determined one or more predicted amino acid-IPC interactions.

82. The system of embodiment 81, wherein the one or more processors are further configured to execute the instructions to:
generating a treatment recommendation that includes the individualized vaccine.

83. The system of embodiment 44, wherein the one or more processors are further configured to execute the instructions to:
selecting a subset of peptides from the set of amino acid sequences to include as a target for an immunotherapy based on the determined one or more predicted amino acid-IPC interactions.

84. The system of embodiment 83, wherein the immunotherapy comprises one or more of: a T cell therapy, a personalized cancer therapy, an antigen-specific immunotherapy, an antigen-dependent immunotherapy, a vaccine, or a natural killer (NK) cell therapy.

85. The system of embodiment 44, wherein the one or more processors are further configured to execute the instructions to:
selecting a subset of peptides from the set of amino acid sequences to exclude as a target for an immunotherapy based on the determined one or more predicted amino acid-IPC interactions.

86. The system of embodiment 85, wherein the immunotherapy comprises one or more of: a T cell therapy, a personalized cancer therapy, an antigen-specific immunotherapy, an antigen-dependent immunotherapy, a vaccine, or a natural killer (NK) cell therapy.

87. A non-transitory computer-readable medium comprising instructions that, when executed by one or more processors of one or more computing devices, cause the one or more processors to:
access a set of amino acid sequences, each of the amino acid sequences of the set having been identified from at least one protein;
access an immunoprotein complex (IPC) sequence identified for an IPC of a subject;
process, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
process, using a second processing block in the processing subsystem, an IPC sequence representation to generate a transformed IPC sequence representation, wherein the set of amino acid sequence representations and the IPC sequence representation are processed in parallel, wherein the IPC sequence representation was generated based on the identified IPC sequence appended with a BOS token;
generate composite representations by combining each of the transformed BOS token representations of the set of transformed amino acid sequence representations with the transformed BOS token representations of the transformed IPC sequence representation; and
determine one or more predicted amino acid-IPC interactions based on the composite representations.

88. The non-transitory computer-readable medium of embodiment 87, wherein the set of transformed amino acid sequence representations comprises a set of amino-terminal flanking (N-flank) representations or a set of carboxy-terminal flanking (C-flank) representations.

89. The non-transitory computer-readable medium of embodiment 87, wherein the IPC of the subject is a major histocompatibility complex (MHC).

90. The non-transitory computer-readable medium of embodiment 89, wherein the set of transformed amino acid sequence representations comprises a set of MHC-binding representations.

91. The non-transitory computer-readable medium of embodiment 89, wherein the MHC comprises MHC class II (MHC-II).

92. The non-transitory computer-readable medium of embodiment 89, wherein the MHC comprises MHC class I (MHC-I).

93. The non-transitory computer-readable medium of embodiment 87, wherein the IPC of the subject is a T-cell receptor (TCR).

94. The non-transitory computer-readable medium of embodiment 87, wherein the at least one protein is a therapeutic protein.

95. The non-transitory computer-readable medium of embodiment 87, wherein the at least one protein is present in a disease sample from the subject.

96. The non-transitory computer-readable medium of embodiment 95, wherein the disease sample is a tumor cell biopsy.

97. The non-transitory computer-readable medium of embodiment 95, wherein the disease sample includes cancer.

98. The non-transitory computer-readable medium of embodiment 95, wherein the disease sample includes tissue.

99. The non-transitory computer-readable medium of embodiment 87, wherein generating composite representations comprises:
for each of the set of transformed amino acid sequence representations, elementwise multiplying a transformed amino acid beginning-of-sequence (BOS) representation corresponding to the transformed amino acid sequence representation by a transformed IPC beginning-of-sequence (BOS) representation corresponding to the transformed IPC sequence representation.

100. The non-transitory computer-readable medium of embodiment 87, wherein processing a set of amino acid sequence representations comprises processing a peptide beginning-of-sequence (BOS) representation to generate a transformed peptide sequence representation.

101. The non-transitory computer-readable medium of embodiment 87, wherein processing an IPC sequence representation comprises processing an MHC beginning-of-sequence representation (BOS) to generate a transformed MHC sequence representation.

102. The non-transitory computer-readable medium of embodiment 87, further comprising instructions that cause the one or more processors to:
for each of a set of IPC sequences, perform the steps of: accessing the IPC sequence, processing the set of amino acid sequence representations, processing the IPC sequence representation using the respective IPC sequence, and generating the composite representations;
wherein the determined one or more predicted amino acid-IPC interactions are based on the composite representations corresponding to the set of IPC sequences.

103. The non-transitory computer-readable medium of embodiment 102, wherein the set of IPC sequences comprises twelve major histocompatibility complex (MHC) allotypes of the subject and/or six MHC alleles of the subject.

104. The non-transitory computer-readable medium of embodiment 102, further comprising instructions that cause the one or more processors to:
select one or more peptide-IPC combinations from the set of amino acid sequences and the set of IPC sequences to include as a target for an immunotherapy based on the one or more determined amino-acid IPC interactions.

105. The non-transitory computer-readable medium of embodiment 87, wherein processing the set of amino acid sequence representations comprises:
transforming the set of amino acid sequence representations using the one or more first processing blocks into the set of transformed amino acid sequence representations, wherein each of the one or more first processing blocks includes a set of processing sub-blocks.

106. The non-transitory computer-readable medium of embodiment 87, further comprising instructions that cause the one or more processors to:
embed the set of amino acid sequences to generate a set of embedded amino acid sequence representations; and
positionally encode the set of embedded amino acid sequence representations.

107. The non-transitory computer-readable medium of embodiment 87, wherein processing the IPC sequence representation comprises:
transforming the IPC sequence representation using the second processing block into a transformed IPC sequence representation, wherein the second processing block includes a set of processing sub-blocks.

108. The non-transitory computer-readable medium of embodiment 87, further comprising instructions that cause the one or more processors to:
embed the IPC sequence to generate an embedded IPC sequence representation; and
positionally encode the embedded IPC sequence representation.

109. The non-transitory computer-readable medium of embodiment 87, wherein the machine-learning model includes one or more transformer encoders, each of the one or more transformer encoders including a processing layer.

110. The non-transitory computer-readable medium of embodiment 87, wherein:
each of the one or more first processing blocks or the second processing block comprises a set of processing sub-blocks; and
each of the set of processing sub-blocks includes a neural network comprising at least one processing layer.

111. The non-transitory computer-readable medium of embodiment 87, wherein the set of amino acid sequence representations comprises aggregate sequence representations, the aggregate sequence representations including a set of peptide representations and one or more of: a set of amino-terminal flanking (N-flank) representations or a set of carboxy-terminal flanking (C-flank) representations.

112. The non-transitory computer-readable medium of embodiment 87, further comprising, prior to generating the set of transformed amino acid sequence representations:
flattening the aggregate sequence representations into a single array; and
densifying the aggregate sequence representations by removing empty rows from the array, wherein the transformed amino acid sequence representations are generated based on the densified aggregate sequence representations.

113. The non-transitory computer-readable medium of embodiment 87, wherein the IPC sequence representation comprises aggregate sequence representations, the aggregate sequence representations including one or more of: a major histocompatibility complex (MHC) sequence representation or a T-cell receptor (TCR) sequence representation.

114. The non-transitory computer-readable medium of embodiment 87, wherein processing the set of amino acid sequence representations comprises:
for each amino acid sequence representation of the set:
   determining, for each element of the IPC sequence representation, a plurality of vectors based on a set of weights associated with a processing layer of the machine-learning model; and
   generating the set of element-focused scores based on the plurality of vectors and the set of weights.

115. The non-transitory computer-readable medium of embodiment 114, wherein the plurality of vectors comprises a key vector, a value vector, and a query vector, and the set of weights comprises a set of key weights, a set of value weights, and a set of query weights.

116. The non-transitory computer-readable medium of embodiment 114, wherein generating the set of element-focused scores comprises:
determining each element-focused score from each pair of elements from the query vector and the key vector.

117. The non-transitory computer-readable medium of embodiment 87, wherein the one or more first processing blocks and the second processing block comprise attention blocks, each attention block comprising a set of attention sub-blocks, each attention sub-block comprising a self-attention layer; and
wherein the machine-learning model is an attention-based machine learning model.

118. The non-transitory computer-readable medium of embodiment 117, further comprising instructions that cause the one or more processors to:
by one or more of the attention blocks, generate attention maps including masks limiting attention applied by the attention sub-blocks to a sequence length according to the masks.

119. The non-transitory computer-readable medium of embodiment 87, further comprising instructions that cause the one or more processors to:
process, using a fully connected block in an output subsystem of the machine-learning model, the composition representations to generate a first output;
apply, using a dropout block in the output subsystem of the machine-learning model, a dropout to the first output to generate a second output; and
select, using a max layer in the output subsystem of the machine-learning model, a subset of the second output to generate a result,
wherein the one or more predicted amino acid-IPC interactions are determined based on the result.

120. The non-transitory computer-readable medium of embodiment 87, wherein the set of amino acid sequences comprises a peptide sequence and the IPC sequence comprises a major histocompatibility complex (MHC) sequence, and
wherein the one or more predicted amino acid-IPC interactions comprise one or more of:
an interaction affinity prediction for a peptide-IPC combination that predicts a binding affinity between a peptide and MHC;
an interaction prediction for the peptide-IPC combination that predicts whether the MHC will present the peptide at a cell surface; or
an immunogenicity prediction for the peptide-IPC combination that predicts an ability of the peptide to provoke an immune response with respect to the MHC.

121. The non-transitory computer-readable medium of embodiment 87, wherein determining the one or more predicted amino acid-IPC interactions comprises:
processing the composite representations to generate a set of results; and
selecting an amino acid-IPC combination based on a highest result among the set of results.

122. The non-transitory computer-readable medium of embodiment 87, wherein the one or more predicted amino acid-IPC interactions comprise a prediction of tumor-specific immunogenicity of a peptide.

123. The non-transitory computer-readable medium of embodiment 87, wherein the set of amino acid sequences comprises a set of peptide sequences, wherein the one or more predicted amino acid-IPC interactions identify a subset of peptide sequences having increased tumor-specific immunogenicity or increased likelihood of presentation by the IPC relative to the set of peptide sequences.

124. The non-transitory computer-readable medium of embodiment 87, further comprising instructions that cause the one or more processors to:
identify a subset of peptides from the set of amino acid sequences to include in an individualized vaccine based on the determined one or more predicted amino acid-IPC interactions.

125. The non-transitory computer-readable medium of embodiment 124, further comprising instructions that cause the one or more processors to:
generate a treatment recommendation that includes the individualized vaccine.

126. The non-transitory computer-readable medium of embodiment 87, further comprising instructions that cause the one or more processors to:
select a subset of peptides from the set of amino acid sequences to include as a target for an immunotherapy based on the determined one or more predicted amino acid-IPC interactions.

127. The non-transitory computer-readable medium of embodiment 126, wherein the immunotherapy comprises one or more of: a T cell therapy, a personalized cancer therapy, an antigen-specific immunotherapy, an antigen-dependent immunotherapy, a vaccine, or a natural killer (NK) cell therapy.

128. The non-transitory computer-readable medium of embodiment 87, further comprising instructions that cause the one or more processors to:
select a subset of peptides from the set of amino acid sequences to exclude as a target for an immunotherapy based on the determined one or more predicted amino acid-IPC interactions.

129. The non-transitory computer-readable medium of embodiment 128, wherein the immunotherapy comprises one or more of: a T cell therapy, a personalized cancer therapy, an antigen-specific immunotherapy, an antigen-dependent immunotherapy, a vaccine, or a natural killer (NK) cell therapy.

130. A vaccine comprising:
one or more peptides;
a plurality of nucleic acids that encode the one or more peptides; or
a plurality of cells expressing the one or more peptides,
wherein the one or more peptides are selected from among a set of peptides by:
   accessing a set of amino acid sequences, each of the amino acid sequences of the set having been identified from at least one protein;
   accessing an immunoprotein complex (IPC) sequence identified for an IPC of a subject;
   processing, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
   processing, using a second processing block in the processing subsystem, an IPC sequence representation to generate a transformed IPC sequence representation, wherein the set of amino acid sequence representations and the IPC sequence representation are processed in parallel, wherein the IPC sequence representation was generated based on the identified IPC sequence appended with a BOS token;
   generating composite representations by combining each of the transformed BOS token representations of the set of transformed amino acid sequence representations with the transformed BOS token representations of the transformed IPC sequence representation;
   determining one or more predicted amino acid-IPC interactions based on the composite representations; and
   selecting one or more amino acid-IPC combinations based on the one or more predicted amino acid-IPC interactions,
   wherein the one or more peptides correspond to the selected one or more amino acid-IPC combinations.

131. The vaccine of embodiment 130, wherein the set of transformed amino acid sequence representations comprises a set of amino-terminal flanking (N-flank) representations or a set of carboxy-terminal flanking (C-flank) representations.

132. The vaccine of embodiment 130, wherein the IPC of the subject is a major histocompatibility complex (MHC).

133. The vaccine of embodiment 132, wherein the set of transformed amino acid sequence representations comprises a set of MHC-binding representations.

134. The vaccine of embodiment 132, wherein the MHC comprises MHC class II (MHC-II).

135. The vaccine of embodiment 132, wherein the MHC comprises MHC class I (MHC-I).

136. The vaccine of embodiment 130, wherein the IPC of the subject is a T-cell receptor (TCR).

137. The vaccine of embodiment 130, wherein the at least one protein is a therapeutic protein.

138. The vaccine of embodiment 130, wherein the at least one protein is present in a disease sample from the subject.

139. The vaccine of embodiment 138, wherein the disease sample is a tumor cell biopsy.

140. The vaccine of embodiment 138, wherein the disease sample includes cancer.

141. The vaccine of embodiment 138, wherein the disease sample includes tissue.

142. The vaccine of embodiment 130, wherein generating composite representations comprises:
for each of the set of transformed amino acid sequence representations, elementwise multiplying a transformed amino acid beginning-of-sequence (BOS) representation corresponding to the transformed amino acid sequence representation by a transformed IPC beginning-of-sequence (BOS) representation corresponding to the transformed IPC sequence representation.

143. The vaccine of embodiment 130, wherein processing a set of amino acid sequence representations comprises processing a peptide beginning-of-sequence (BOS) representation to generate a transformed peptide sequence representation.

144. The vaccine of embodiment 130, wherein processing an IPC sequence representation comprises processing an MHC beginning-of-sequence representation (BOS) to generate a transformed MHC sequence representation.

145. The vaccine of embodiment 130, wherein the one or more peptides are selected from among the set of peptides by further:
for each of a set of IPC sequences, performing the steps of: accessing the IPC sequence, processing the set of amino acid sequence representations, processing the IPC sequence representation using the respective IPC sequence, and generating the composite representations;
wherein the determined one or more predicted amino acid-IPC interactions are based on the composite representations corresponding to the set of IPC sequences.

146. The vaccine of embodiment 145, wherein the set of IPC sequences comprises twelve major histocompatibility complex (MHC) allotypes of the subject and/or six MHC alleles of the subject.

147. The vaccine of embodiment 145, wherein the one or more peptides are selected from among the set of peptides by further:
selecting one or more peptide-IPC combinations from the set of amino acid sequences and the set of IPC sequences to include as a target for an immunotherapy based on the one or more determined amino-acid IPC interactions.

148. The vaccine of embodiment 130, wherein processing the set of amino acid sequence representations comprises:
transforming the set of amino acid sequence representations using the one or more first processing blocks into the set of transformed amino acid sequence representations, wherein each of the one or more first processing blocks includes a set of processing sub-blocks.

149. The vaccine of embodiment 130, wherein the one or more peptides are selected from among the set of peptides by further:
embedding the set of amino acid sequences to generate a set of embedded amino acid sequence representations; and
positionally encoding the set of embedded amino acid sequence representations.

150. The vaccine of embodiment 130, wherein processing the IPC sequence representation comprises:
transforming the IPC sequence representation using the second processing block into a transformed IPC sequence representation, wherein the second processing block includes a set of processing sub-blocks.

151. The vaccine of embodiment 130, wherein the one or more peptides are selected from among the set of peptides by further:
embedding the IPC sequence to generate an embedded IPC sequence representation; and
positionally encoding the embedded IPC sequence representation.

152. The vaccine of embodiment 130, wherein the machine-learning model includes one or more transformer encoders, each of the one or more transformer encoders including a processing layer.

153. The vaccine of embodiment 130, wherein:
each of the one or more first processing blocks or the second processing block comprises a set of processing sub-blocks; and
each of the set of processing sub-blocks includes a neural network comprising at least one processing layer.

154. The vaccine of embodiment 130, wherein the set of amino acid sequence representations comprises aggregate sequence representations, the aggregate sequence representations including a set of peptide representations and one or more of: a set of amino-terminal flanking (N-flank) representations or a set of carboxy-terminal flanking (C-flank) representations.

155. The vaccine of embodiment 130, wherein the one or more peptides are selected from among the set of peptides by, prior to generating the set of transformed amino acid sequence representations:
flattening the aggregate sequence representations into a single array; and
densifying the aggregate sequence representations by removing empty rows from the array, wherein the transformed amino acid sequence representations are generated based on the densified aggregate sequence representations.

156. The vaccine of embodiment 130, wherein the IPC sequence representation comprises aggregate sequence representations, the aggregate sequence representations including one or more of: a major histocompatibility complex (MHC) sequence representation or a T-cell receptor (TCR) sequence representation.

157. The vaccine of embodiment 130, wherein processing the set of amino acid sequence representations comprises:
for each amino acid sequence representation of the set:
   determining, for each element of the IPC sequence representation, a plurality of vectors based on a set of weights associated with a processing layer of the machine-learning model; and
   generating the set of element-focused scores based on the plurality of vectors and the set of weights.

158. The vaccine of embodiment 157, wherein the plurality of vectors comprises a key vector, a value vector, and a query vector, and the set of weights comprises a set of key weights, a set of value weights, and a set of query weights.

159. The vaccine of embodiment 157, wherein generating the set of element-focused scores comprises:
determining each element-focused score from each pair of elements from the query vector and the key vector.

160. The vaccine of embodiment 130, wherein the one or more first processing blocks and the second processing block comprise attention blocks, each attention block comprising a set of attention sub-blocks, each attention sub-block comprising a self-attention layer; and
wherein the machine-learning model is an attention-based machine learning model.

161. The vaccine of embodiment 160, wherein the one or more peptides are selected from among the set of peptides by further:
by one or more of the attention blocks, generating attention maps including masks limiting attention applied by the attention sub-blocks to a sequence length according to the masks.

162. The vaccine of embodiment 130, wherein the one or more peptides are selected from among the set of peptides by further:
processing, using a fully connected block in an output subsystem of the machine-learning model, the composition representations to generate a first output;
applying, using a dropout block in the output subsystem of the machine-learning model, a dropout to the first output to generate a second output; and
selecting, using a max layer in the output subsystem of the machine-learning model, a subset of the second output to generate a result,
wherein the one or more predicted amino acid-IPC interactions are determined based on the result.

163. The vaccine of embodiment 130, wherein the set of amino acid sequences comprises a peptide sequence and the IPC sequence comprises a major histocompatibility complex (MHC) sequence, and
wherein the one or more predicted amino acid-IPC interactions comprise one or more of:
an interaction affinity prediction for a peptide-IPC combination that predicts a binding affinity between a peptide and MHC;
an interaction prediction for the peptide-IPC combination that predicts whether the MHC will present the peptide at a cell surface; or
an immunogenicity prediction for the peptide-IPC combination that predicts an ability of the peptide to provoke an immune response with respect to the MHC.

164. The vaccine of embodiment 130, wherein determining the one or more predicted amino acid-IPC interactions comprises:
processing the composite representations to generate a set of results; and
selecting an amino acid-IPC combination based on a highest result among the set of results.

165. The vaccine of embodiment 130, wherein the one or more predicted amino acid-IPC interactions comprise a prediction of tumor-specific immunogenicity of a peptide.

166. The vaccine of embodiment 130, wherein the set of amino acid sequences comprises a set of peptide sequences, wherein the one or more predicted amino acid-IPC interactions identify a subset of peptide sequences having increased tumor-specific immunogenicity or increased likelihood of presentation by the IPC relative to the set of peptide sequences.

167. The vaccine of embodiment 130, wherein the one or more peptides are selected from among the set of peptides by further:
identifying a subset of peptides from the set of amino acid sequences to include in an individualized vaccine based on the determined one or more predicted amino acid-IPC interactions.

168. The vaccine of embodiment 167, wherein the one or more peptides are selected from among the set of peptides by further:
generating a treatment recommendation that includes the individualized vaccine.

169. The vaccine of embodiment 130, wherein the one or more peptides are selected from among the set of peptides by further:
selecting a subset of peptides from the set of amino acid sequences to include as a target for an immunotherapy based on the determined one or more predicted amino acid-IPC interactions.

170. The vaccine of embodiment 169, wherein the immunotherapy comprises one or more of: a T cell therapy, a personalized cancer therapy, an antigen-specific immunotherapy, an antigen-dependent immunotherapy, a vaccine, or a natural killer (NK) cell therapy.

171. The vaccine of embodiment 130, wherein the one or more peptides are selected from among the set of peptides by further:
selecting a subset of peptides from the set of amino acid sequences to exclude as a target for an immunotherapy based on the determined one or more predicted amino acid-IPC interactions.

172. The vaccine of embodiment 171, wherein the immunotherapy comprises one or more of: a T cell therapy, a personalized cancer therapy, an antigen-specific immunotherapy, an antigen-dependent immunotherapy, a vaccine, or a natural killer (NK) cell therapy.

173. A method of manufacturing a vaccine comprising:
producing a vaccine comprising:
one or more peptides;
a plurality of nucleic acids that encode the one or more peptides; or
a plurality of cells expressing the one or more peptides,
wherein the one or more peptides are selected from among a set of peptides by:
accessing a set of amino acid sequences, each of the amino acid sequences of the set having been identified from at least one protein;
accessing an immunoprotein complex (IPC) sequence identified for an IPC of a subject;
processing, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
processing, using a second processing block in the processing subsystem, an IPC sequence representation to generate a transformed IPC sequence representation, wherein the set of amino acid sequence representations and the IPC sequence representation are processed in parallel, wherein the IPC sequence representation was generated based on the identified IPC sequence appended with a BOS token;
generating composite representations by combining each of the transformed BOS token representations of the set of transformed amino acid sequence representations with the transformed BOS token representations of the transformed IPC sequence representation;
determining one or more predicted amino acid-IPC interactions based on the composite representations; and
selecting one or more amino acid-IPC combinations based on the one or more predicted amino-acid IPC interactions,
wherein the one or more peptides correspond to the selected one or more amino acid-IPC combinations.

174. The method of embodiment 173, wherein the set of transformed amino acid sequence representations comprises a set of amino-terminal flanking (N-flank) representations or a set of carboxy-terminal flanking (C-flank) representations.

175. The method of embodiment 173, wherein the IPC of the subject is a major histocompatibility complex (MHC).

176. The method of embodiment 175, wherein the set of transformed amino acid sequence representations comprises a set of MHC-binding representations.

177. The method of embodiment 175, wherein the MHC comprises MHC class II (MHC-II).

178. The method of embodiment 175, wherein the MHC comprises MHC class I (MHC-I).

179. The method of embodiment 173, wherein the IPC of the subject is a T-cell receptor (TCR).

180. The method of embodiment 173, wherein the at least one protein is a therapeutic protein.

181. The method of embodiment 173, wherein the at least one protein is present in a disease sample from the subject.

182. The method of embodiment 181, wherein the disease sample is a tumor cell biopsy.

183. The method of embodiment 181, wherein the disease sample includes cancer.

184. The method of embodiment 181, wherein the disease sample includes tissue.

185. The method of embodiment 173, wherein generating composite representations comprises:
for each of the set of transformed amino acid sequence representations, elementwise multiplying a transformed amino acid beginning-of-sequence (BOS) representation corresponding to the transformed amino acid sequence representation by a transformed IPC beginning-of-sequence (BOS) representation corresponding to the transformed IPC sequence representation.

186. The method of embodiment 173, wherein processing a set of amino acid sequence representations comprises processing a peptide beginning-of-sequence (BOS) representation to generate a transformed peptide sequence representation.

187. The method of embodiment 173, wherein processing an IPC sequence representation comprises processing an MHC beginning-of-sequence representation (BOS) to generate a transformed MHC sequence representation.

188. The method of embodiment 173, wherein the one or more peptides are selected from among the set of peptides by further:
for each of a set of IPC sequences, performing the steps of: accessing the IPC sequence, processing the set of amino acid sequence representations, processing the IPC sequence representation using the respective IPC sequence, and generating the composite representations;
wherein the determined one or more predicted amino acid-IPC interactions are based on the composite representations corresponding to the set of IPC sequences.

189. The method of embodiment 188, wherein the set of IPC sequences comprises twelve major histocompatibility complex (MHC) allotypes of the subject and/or six MHC alleles of the subject.

190. The method of embodiment 188, wherein the one or more peptides are selected from among the set of peptides by further:
selecting one or more peptide-IPC combinations from the set of amino acid sequences and the set of IPC sequences to include as a target for an immunotherapy based on the one or more determined amino-acid IPC interactions.

191. The method of embodiment 173, wherein processing the set of amino acid sequence representations comprises:
transforming the set of amino acid sequence representations using the one or more first processing blocks into the set of transformed amino acid sequence representations, wherein each of the one or more first processing blocks includes a set of processing sub-blocks.

192. The method of embodiment 173, wherein the one or more peptides are selected from among the set of peptides by further:
embedding the set of amino acid sequences to generate a set of embedded amino acid sequence representations; and
positionally encoding the set of embedded amino acid sequence representations.

193. The method of embodiment 173, wherein processing the IPC sequence representation comprises:
transforming the IPC sequence representation using the second processing block into a transformed IPC sequence representation, wherein the second processing block includes a set of processing sub-blocks.

194. The method of embodiment 173, wherein the one or more peptides are selected from among the set of peptides by further:
embedding the IPC sequence to generate an embedded IPC sequence representation; and
positionally encoding the embedded IPC sequence representation.

195. The method of embodiment 173, wherein the machine-learning model includes one or more transformer encoders, each of the one or more transformer encoders including a processing layer.

196. The method of embodiment 173, wherein:
each of the one or more first processing blocks or the second processing block comprises a set of processing sub-blocks; and
each of the set of processing sub-blocks includes a neural network comprising at least one processing layer.

197. The method of embodiment 173, wherein the set of amino acid sequence representations comprises aggregate sequence representations, the aggregate sequence representations including a set of peptide representations and one or more of: a set of amino-terminal flanking (N-flank) representations or a set of carboxy-terminal flanking (C-flank) representations.

198. The method of embodiment 173, wherein the one or more peptides are selected from among the set of peptides by further, prior to generating the set of transformed amino acid sequence representations:
flattening the aggregate sequence representations into a single array; and
densifying the aggregate sequence representations by removing empty rows from the array, wherein the transformed amino acid sequence representations are generated based on the densified aggregate sequence representations.

199. The method of embodiment 173, wherein the IPC sequence representation comprises aggregate sequence representations, the aggregate sequence representations including one or more of: a major histocompatibility complex (MHC) sequence representation or a T-cell receptor (TCR) sequence representation.

200. The method of embodiment 173, wherein processing the set of amino acid sequence representations comprises:
for each amino acid sequence representation of the set:
   determining, for each element of the IPC sequence representation, a plurality of vectors based on a set of weights associated with a processing layer of the machine-learning model; and
   generating the set of element-focused scores based on the plurality of vectors and the set of weights.

201. The method of embodiment 200, wherein the plurality of vectors comprises a key vector, a value vector, and a query vector, and the set of weights comprises a set of key weights, a set of value weights, and a set of query weights.

202. The method of embodiment 200, wherein generating the set of element-focused scores comprises:
determining each element-focused score from each pair of elements from the query vector and the key vector.

203. The method of embodiment 173, wherein the one or more first processing blocks and the second processing block comprise attention blocks, each attention block comprising a set of attention sub-blocks, each attention sub-block comprising a self-attention layer; and
wherein the machine-learning model is an attention-based machine learning model.

204. The method of embodiment 203, wherein the one or more peptides are selected from among the set of peptides by further:
by one or more of the attention blocks, generating attention maps including masks limiting attention applied by the attention sub-blocks to a sequence length according to the masks.

205. The method of embodiment 173, wherein the one or more peptides are selected from among the set of peptides by further:
processing, using a fully connected block in an output subsystem of the machine-learning model, the composition representations to generate a first output;
applying, using a dropout block in the output subsystem of the machine-learning model, a dropout to the first output to generate a second output; and
selecting, using a max layer in the output subsystem of the machine-learning model, a subset of the second output to generate a result,
wherein the one or more predicted amino acid-IPC interactions are determined based on the result.

206. The method of embodiment 173, wherein the set of amino acid sequences comprises a peptide sequence and the IPC sequence comprises a major histocompatibility complex (MHC) sequence, and
wherein the one or more predicted amino acid-IPC interactions comprise one or more of:
an interaction affinity prediction for a peptide-IPC combination that predicts a binding affinity between a peptide and MHC;
an interaction prediction for the peptide-IPC combination that predicts whether the MHC will present the peptide at a cell surface; or
an immunogenicity prediction for the peptide-IPC combination that predicts an ability of the peptide to provoke an immune response with respect to the MHC.

207. The method of embodiment 173, wherein determining the one or more predicted amino acid-IPC interactions comprises:
processing the composite representations to generate a set of results; and
selecting an amino acid-IPC combination based on a highest result among the set of results.

208. The method of embodiment 173, wherein the one or more predicted amino acid-IPC interactions comprise a prediction of tumor-specific immunogenicity of a peptide.

209. The method of embodiment 173, wherein the set of amino acid sequences comprises a set of peptide sequences, wherein the one or more predicted amino acid-IPC interactions identify a subset of peptide sequences having increased tumor-specific immunogenicity or increased likelihood of presentation by the IPC relative to the set of peptide sequences.

210. The method of embodiment 173, wherein the one or more peptides are selected from among the set of peptides by further:
identifying a subset of peptides from the set of amino acid sequences to include in an individualized vaccine based on the determined one or more predicted amino acid-IPC interactions.

211. The method of embodiment 210, wherein the one or more peptides are selected from among the set of peptides by further:
generating a treatment recommendation that includes the individualized vaccine.

212. The method of embodiment 173, wherein the one or more peptides are selected from among the set of peptides by further:
selecting a subset of peptides from the set of amino acid sequences to include as a target for an immunotherapy based on the determined one or more predicted amino acid-IPC interactions.

213. The method of embodiment 212, wherein the immunotherapy comprises one or more of: a T cell therapy, a personalized cancer therapy, an antigen-specific immunotherapy, an antigen-dependent immunotherapy, a vaccine, or a natural killer (NK) cell therapy.

214. The method of embodiment 173, wherein the one or more peptides are selected from among the set of peptides by further:
selecting a subset of peptides from the set of amino acid sequences to exclude as a target for an immunotherapy based on the determined one or more predicted amino acid-IPC interactions.

215. The method of embodiment 214, wherein the immunotherapy comprises one or more of: a T cell therapy, a personalized cancer therapy, an antigen-specific immunotherapy, an antigen-dependent immunotherapy, a vaccine, or a natural killer (NK) cell therapy.

216. A pharmaceutical composition comprising one or more peptides selected from among a set of peptides by:
accessing a set of amino acid sequences, each of the amino acid sequences of the set having been identified from at least one protein;
accessing an immunoprotein complex (IPC) sequence identified for an IPC of a subject;
processing, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
processing, using a second processing block in the processing subsystem, an IPC sequence representation to generate a transformed IPC sequence representation, wherein the set of amino acid sequence representations and the IPC sequence representation are processed in parallel, wherein the IPC sequence representation was generated based on the identified IPC sequence appended with a BOS token;
generating composite representations by combining each of the transformed BOS token representations of the set of transformed amino acid sequence representations with the transformed BOS token representations of the transformed IPC sequence representation;
determining one or more predicted amino acid-IPC interactions based on the composite representations; and
selecting one or more amino acid-IPC combinations based on the one or more predicted amino acid-IPC interactions,
wherein the one or more peptides correspond to the selected one or more amino acid-IPC combinations.

217. The pharmaceutical composition of embodiment 216, wherein the set of transformed amino acid sequence representations comprises a set of amino-terminal flanking (N-flank) representations or a set of carboxy-terminal flanking (C-flank) representations.

218. The pharmaceutical composition of embodiment 216, wherein the IPC of the subject is a major histocompatibility complex (MHC).

219. The pharmaceutical composition of embodiment 218, wherein the set of transformed amino acid sequence representations comprises a set of MHC-binding representations.

220. The pharmaceutical composition of embodiment 218, wherein the MHC comprises MHC class II (MHC-II).

221. The pharmaceutical composition of embodiment 218, wherein the MHC comprises MHC class I (MHC-I).

222. The pharmaceutical composition of embodiment 216, wherein the IPC of the subject is a T-cell receptor (TCR).

223. The pharmaceutical composition of embodiment 216, wherein the at least one protein is a therapeutic protein.

224. The pharmaceutical composition of embodiment 216, wherein the at least one protein is present in a disease sample from the subject.

225. The pharmaceutical composition of embodiment 224, wherein the disease sample is a tumor cell biopsy.

226. The pharmaceutical composition of embodiment 224, wherein the disease sample includes cancer.

227. The pharmaceutical composition of embodiment 224, wherein the disease sample includes tissue.

228. The pharmaceutical composition of embodiment 216, wherein generating composite representations comprises:
for each of the set of transformed amino acid sequence representations, elementwise multiplying a transformed amino acid beginning-of-sequence (BOS) representation corresponding to the transformed amino acid sequence representation by a transformed IPC beginning-of-sequence (BOS) representation corresponding to the transformed IPC sequence representation.

229. The pharmaceutical composition of embodiment 216, wherein processing a set of amino acid sequence representations comprises processing a peptide beginning-of-sequence (BOS) representation to generate a transformed peptide sequence representation.

230. The pharmaceutical composition of embodiment 216, wherein processing an IPC sequence representation comprises processing an MHC beginning-of-sequence representation (BOS) to generate a transformed MHC sequence representation.

231. The pharmaceutical composition of embodiment 216, wherein the one or more peptides are selected from among the set of peptides by further:
for each of a set of IPC sequences, performing the steps of: accessing the IPC sequence, processing the set of amino acid sequence representations, processing the IPC sequence representation using the respective IPC sequence, and generating the composite representations;
wherein the determined one or more predicted amino acid-IPC interactions are based on the composite representations corresponding to the set of IPC sequences.

232. The pharmaceutical composition of embodiment 231, wherein the set of IPC sequences comprises twelve major histocompatibility complex (MHC) allotypes of the subject and/or six MHC alleles of the subject.

233. The pharmaceutical composition of embodiment 231, wherein the one or more peptides are selected from among the set of peptides by further:
selecting one or more peptide-IPC combinations from the set of amino acid sequences and the set of IPC sequences to include as a target for an immunotherapy based on the one or more determined amino-acid IPC interactions.

234. The pharmaceutical composition of embodiment 216, wherein processing the set of amino acid sequence representations comprises:
transforming the set of amino acid sequence representations using the one or more first processing blocks into the set of transformed amino acid sequence representations, wherein each of the one or more first processing blocks includes a set of processing sub-blocks.

235. The pharmaceutical composition of embodiment 216, wherein the one or more peptides are selected from among the set of peptides by further:
embedding the set of amino acid sequences to generate a set of embedded amino acid sequence representations; and
positionally encoding the set of embedded amino acid sequence representations.

236. The pharmaceutical composition of embodiment 216, wherein processing the IPC sequence representation comprises:
transforming the IPC sequence representation using the second processing block into a transformed IPC sequence representation, wherein the second processing block includes a set of processing sub-blocks.

237. The pharmaceutical composition of embodiment 216, wherein the one or more peptides are selected from among the set of peptides by further:
embedding the IPC sequence to generate an embedded IPC sequence representation; and
positionally encoding the embedded IPC sequence representation.

238. The pharmaceutical composition of embodiment 216, wherein the machine-learning model includes one or more transformer encoders, each of the one or more transformer encoders including a processing layer.

239. The pharmaceutical composition of embodiment 216, wherein:
each of the one or more first processing blocks or the second processing block comprises a set of processing sub-blocks; and
each of the set of processing sub-blocks includes a neural network comprising at least one processing layer.

240. The pharmaceutical composition of embodiment 216, wherein the set of amino acid sequence representations comprises aggregate sequence representations, the aggregate sequence representations including a set of peptide representations and one or more of: a set of amino-terminal flanking (N-flank) representations or a set of carboxy-terminal flanking (C-flank) representations.

241. The pharmaceutical composition of embodiment 216, wherein the one or more peptides are selected from among the set of peptides by further, prior to generating the set of transformed amino acid sequence representations:
flattening the aggregate sequence representations into a single array; and
densifying the aggregate sequence representations by removing empty rows from the array, wherein the transformed amino acid sequence representations are generated based on the densified aggregate sequence representations.

242. The pharmaceutical composition of embodiment 216, wherein the IPC sequence representation comprises aggregate sequence representations, the aggregate sequence representations including one or more of: a major histocompatibility complex (MHC) sequence representation or a T-cell receptor (TCR) sequence representation.

243. The pharmaceutical composition of embodiment 216, wherein processing the set of amino acid sequence representations comprises:
for each amino acid sequence representation of the set:
   determining, for each element of the IPC sequence representation, a plurality of vectors based on a set of weights associated with a processing layer of the machine-learning model; and
   generating the set of element-focused scores based on the plurality of vectors and the set of weights.

244. The pharmaceutical composition of embodiment 243, wherein the plurality of vectors comprises a key vector, a value vector, and a query vector, and the set of weights comprises a set of key weights, a set of value weights, and a set of query weights.

245. The pharmaceutical composition of embodiment 243, wherein generating the set of element-focused scores comprises:
determining each element-focused score from each pair of elements from the query vector and the key vector.

246. The pharmaceutical composition of embodiment 216, wherein the one or more first processing blocks and the second processing block comprise attention blocks, each attention block comprising a set of attention sub-blocks, each attention sub-block comprising a self-attention layer; and
wherein the machine-learning model is an attention-based machine learning model.

247. The pharmaceutical composition of embodiment 246, wherein the one or more peptides are selected from among the set of peptides by further:
by one or more of the attention blocks, generating attention maps including masks limiting attention applied by the attention sub-blocks to a sequence length according to the masks.

248. The pharmaceutical composition of embodiment 216, wherein the one or more peptides are selected from among the set of peptides by further:
processing, using a fully connected block in an output subsystem of the machine-learning model, the composition representations to generate a first output;
applying, using a dropout block in the output subsystem of the machine-learning model, a dropout to the first output to generate a second output; and
selecting, using a max layer in the output subsystem of the machine-learning model, a subset of the second output to generate a result,
wherein the one or more predicted amino acid-IPC interactions are determined based on the result.

249. The pharmaceutical composition of embodiment 216, wherein the set of amino acid sequences comprises a peptide sequence and the IPC sequence comprises a major histocompatibility complex (MHC) sequence, and
wherein the one or more predicted amino acid-IPC interactions comprise one or more of:
an interaction affinity prediction for a peptide-IPC combination that predicts a binding affinity between a peptide and MHC;
an interaction prediction for the peptide-IPC combination that predicts whether the MHC will present the peptide at a cell surface; or
an immunogenicity prediction for the peptide-IPC combination that predicts an ability of the peptide to provoke an immune response with respect to the MHC.

250. The pharmaceutical composition of embodiment 216, wherein determining the one or more predicted amino acid-IPC interactions comprises:
processing the composite representations to generate a set of results; and
selecting an amino acid-IPC combination based on a highest result among the set of results.

251. The pharmaceutical composition of embodiment 216, wherein the one or more predicted amino acid-IPC interactions comprise a prediction of tumor-specific immunogenicity of a peptide.

252. The pharmaceutical composition of embodiment 216, wherein the set of amino acid sequences comprises a set of peptide sequences, wherein the one or more predicted amino acid-IPC interactions identify a subset of peptide sequences having increased tumor-specific immunogenicity or increased likelihood of presentation by the IPC relative to the set of peptide sequences.

253. The pharmaceutical composition of embodiment 216, wherein the one or more peptides are selected from among the set of peptides by further:
identifying a subset of peptides from the set of amino acid sequences to include in an individualized vaccine based on the determined one or more predicted amino acid-IPC interactions.

254. The pharmaceutical composition of embodiment 253, wherein the one or more peptides are selected from among the set of peptides by further:
generating a treatment recommendation that includes the individualized vaccine.

255. The pharmaceutical composition of embodiment 216, wherein the one or more peptides are selected from among the set of peptides by further:
selecting a subset of peptides from the set of amino acid sequences to include as a target for an immunotherapy based on the determined one or more predicted amino acid-IPC interactions.

256. The pharmaceutical composition of embodiment 255, wherein the immunotherapy comprises one or more of: a T cell therapy, a personalized cancer therapy, an antigen-specific immunotherapy, an antigen-dependent immunotherapy, a vaccine, or a natural killer (NK) cell therapy.

257. The pharmaceutical composition of embodiment 216, wherein the one or more peptides are selected from among the set of peptides by further:
selecting a subset of peptides from the set of amino acid sequences to exclude as a target for an immunotherapy based on the determined one or more predicted amino acid-IPC interactions.

258. The pharmaceutical composition of embodiment 257, wherein the immunotherapy comprises one or more of: a T cell therapy, a personalized cancer therapy, an antigen-specific immunotherapy, an antigen-dependent immunotherapy, a vaccine, or a natural killer (NK) cell therapy.

259. The computer-implemented method of embodiment 32, the method further comprising:
calculating a plurality of average attention values corresponding to a plurality of peptide positions by calculating, based on a set of peptides with a uniform length distribution, an average attention value at each peptide position of the plurality of peptide positions; and
subtracting the plurality of average attention values from a mask of the one or more masks.

260. The computer-implemented method of embodiment 1, further comprising obtaining a dataset for training the machine-learning model by:
generating, for a plurality of training peptides, a plurality of transformed peptide representations;
obtaining, for each training peptide, a corresponding cluster of training peptides based on the plurality of transformed peptide representations;
calculating, for each training peptide, an information content based on the corresponding cluster of training peptides; and
excluding one or more training peptides from the training data based on corresponding information contents of the one or more training peptides.

261. The computer-implemented method of embodiment 1, further comprising:
accessing a protein sequence corresponding to the at least one protein;
obtaining a protein sequence embedding based on the protein sequence; and
determining the one or more predicted amino acid-IPC interactions based at least partially on the protein sequence embedding.

262. The computer-implemented method of embodiment 1, wherein the protein language model comprises a pretrained protein language model.

263. The computer-implemented method of embodiment 261, further comprising:
reducing a dimensionality of the protein sequence embedding; and
combining each of the transformed BOS token representations of the set of transformed amino acid sequence representations with the transformed BOS token representation of the transformed IPC sequence representation and the dimensionality-reduced protein sequence embedding.

264. The computer-implemented method of embodiment 263, wherein the dimensionality of the protein sequence embedding is reduced via a neural network.

265. A computer-implemented method for predicting an amino acid-immunoprotein complex (IPC) interaction comprising:
accessing a set of amino acid sequences, each of the amino acid sequences of the set having been identified from at least one protein;
accessing an immunoprotein complex (IPC) sequence identified for an IPC of a subject;
processing, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
processing, using a second processing block in the processing subsystem, an IPC sequence representation to generate a transformed IPC sequence representation, wherein the IPC sequence representation was generated based on the identified IPC sequence appended with a BOS token, and wherein the set of amino acid sequence representations and the IPC sequence representation are processed in parallel;
generating composite representations by combining each of the transformed BOS token representations of the set of transformed amino acid sequence representations with the transformed BOS token representation of the transformed IPC sequence representation; and
determining one or more predicted amino acid-IPC interactions based on the composite representations.

266. The computer-implemented method of embodiment 265, wherein the set of transformed amino acid sequence representations comprises a set of amino-terminal flanking (N-flank) representations or a set of carboxy-terminal flanking (C-flank) representations.

267. The computer-implemented method of embodiment 265, wherein the IPC of the subject is a major histocompatibility complex (MHC) comprising MHC Class I (MHC-I) and/or MHC Class II (MHC-II) or is a T-cell receptor (TCR), and wherein the at least one protein is a therapeutic protein or is present in a disease sample from the subject.

268. The computer-implemented method of embodiment 265, wherein generating composite representations comprises:
for each of the set of transformed amino acid sequence representations, elementwise multiplying a transformed amino acid beginning-of-sequence (BOS) representation corresponding to the transformed amino acid sequence representation by a transformed IPC beginning-of-sequence (BOS) representation corresponding to the transformed IPC sequence representation.

269. The computer-implemented method of embodiment 265, wherein processing a set of amino acid sequence representations comprises processing a peptide beginning-of-sequence (BOS) representation to generate a transformed peptide sequence representation, and wherein processing an IPC sequence representation comprises processing an MHC beginning-of-sequence representation (BOS) to generate a transformed MHC sequence representation.

270. The computer-implemented method of embodiment 265, wherein processing the set of amino acid sequence representations comprises:
transforming the set of amino acid sequence representations using the one or more first processing blocks into the set of transformed amino acid sequence representations, wherein each of the one or more first processing blocks includes a set of processing sub-blocks.

271. The computer-implemented method of embodiment 265, wherein processing the IPC sequence representation comprises:
transforming the IPC sequence representation using the second processing block into a transformed IPC sequence representation, wherein the second processing block includes a set of processing sub-blocks.

272. The computer-implemented method of embodiment 265, wherein the machine-learning model includes one or more transformer encoders, each of the one or more transformer encoders including a processing layer.

273. The computer-implemented method of embodiment 265, wherein the set of amino acid sequence representations comprises aggregate sequence representations, the aggregate sequence representations including a set of peptide representations and one or more of: a set of amino-terminal flanking (N-flank) representations or a set of carboxy-terminal flanking (C-flank) representations.

274. The computer-implemented method of embodiment 265, further comprising, prior to generating the set of transformed amino acid sequence representations:
flattening the aggregate sequence representations into a single array; and
densifying the aggregate sequence representations by removing empty rows from the array, wherein the transformed amino acid sequence representations are generated based on the densified aggregate sequence representations.

275. The computer-implemented method of embodiment 265, wherein processing the set of amino acid sequence representations comprises:
for each amino acid sequence representation of the set:
   determining, for each element of the amino acid sequence representation, a plurality of vectors based on a set of weights associated with a processing layer of the machine-learning model; and
   generating the set of element-focused scores based on the plurality of vectors and the set of weights.

276. The computer-implemented method of embodiment 265, wherein the one or more first processing blocks and the second processing block comprise attention blocks, each attention block comprising a set of attention sub-blocks, each attention sub-block comprising a self-attention layer; and
wherein the machine-learning model is an attention-based machine learning model, and
wherein the method further comprises: by one or more of the attention blocks, generating attention maps including one or more masks limiting attention applied by the attention sub-blocks to a sequence length according to the masks.

277. The computer-implemented method of embodiment 276, the method further comprising:
calculating a plurality of average attention values corresponding to a plurality of peptide positions by calculating, based on a set of peptides with a uniform length distribution, an average attention value at each peptide position of the plurality of peptide positions; and
subtracting the plurality of average attention values from a mask of the one or more masks.

278. The computer-implemented method of embodiment 265, further comprising obtaining a dataset for training the machine-learning model by:
generating, for a plurality of training peptides, a plurality of transformed peptide representations;
obtaining, for each training peptide, a corresponding cluster of training peptides based on the plurality of transformed peptide representations;
calculating, for each training peptide, an information content based on the corresponding cluster of training peptides; and
excluding one or more training peptides from the training data based on corresponding information contents of the one or more training peptides.

279. The computer-implemented method of embodiment 265, further comprising:
processing, using a fully connected block in an output subsystem of the machine-learning model, the composition representations to generate a first output;
applying, using a dropout block in the output subsystem of the machine-learning model, a dropout to the first output to generate a second output; and
selecting, using a max layer in the output subsystem of the machine-learning model, a subset of the second output to generate a result,
wherein the one or more predicted amino acid-IPC interactions are determined based on the result.

280. The computer-implemented method of embodiment 265, wherein the set of amino acid sequences comprises a peptide sequence and the IPC sequence comprises a major histocompatibility complex (MHC) sequence, and
wherein the one or more predicted amino acid-IPC interactions comprise one or more of:
   an interaction affinity prediction for a peptide-IPC combination that predicts a binding affinity between a peptide and MHC;
   an interaction prediction for the peptide-IPC combination that predicts whether the MHC will present the peptide at a cell surface; or
   an immunogenicity prediction for the peptide-IPC combination that predicts an ability of the peptide to provoke an immune response with respect to the MHC.

281. The computer-implemented method of embodiment 265, wherein determining the one or more predicted amino acid-IPC interactions comprises:
processing the composite representations to generate a set of results; and
selecting an amino acid-IPC combination based on a highest result among the set of results.

282. The computer-implemented method of embodiment 265, wherein the one or more predicted amino acid-IPC interactions comprise a prediction of tumor-specific immunogenicity of a peptide.

283. The computer-implemented method of embodiment 265, wherein the set of amino acid sequences comprises a set of peptide sequences, wherein the one or more predicted amino acid-IPC interactions identify a subset of peptide sequences having increased tumor-specific immunogenicity or increased likelihood of presentation by the IPC relative to the set of peptide sequences.

284. The computer-implemented method of embodiment 265, further comprising:
identifying a subset of peptides from the set of amino acid sequences to include in an individualized vaccine, to include as a target for an immunotherapy, and/or to exclude as a target for an immunotherapy based on the determined one or more predicted amino acid-IPC interactions.

285. The computer-implemented method of embodiment 265, further comprising:
accessing a protein sequence corresponding to the at least one protein;
obtaining a protein sequence embedding based on the protein sequence; and
determining the one or more predicted amino acid-IPC interactions based at least partially on the protein sequence embedding.

286. The computer-implemented method of embodiment 265, wherein the protein language model comprises a pretrained protein language model.

287. The computer-implemented method of embodiment 286, further comprising:
reducing a dimensionality of the protein sequence embedding; and
   combining each of the transformed BOS token representations of the set of transformed amino acid sequence representations with the transformed BOS token representation of the transformed IPC sequence representation and the dimensionality-reduced protein sequence embedding.

288. The computer-implemented method of embodiment 287, wherein the dimensionality of the protein sequence embedding is reduced via a neural network.

289. A system for predicting an amino acid-immunoprotein complex (IPC) interaction, comprising:
one or more non-transitory computer-readable storage media including instructions; and
one or more processors coupled to the one or more storage media, the one or more processors configured to execute the instructions to:
   access a set of amino acid sequences, each of the amino acid sequences of the set having been identified from at least one protein;
   access an immunoprotein complex (IPC) sequence identified for an IPC of a subject;
   process, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
   process, using a second processing block in the processing subsystem, an IPC sequence representation to generate a transformed IPC sequence representation, wherein the IPC sequence representation was generated based on the identified IPC sequence appended with a BOS token, and wherein the set of amino acid sequence representations and the IPC sequence representation are processed in parallel;
   generate composite representations by combining each of the transformed BOS token representations of the set of transformed amino acid sequence representations with the transformed BOS token representation of the transformed IPC sequence representation; and
   determine one or more predicted amino acid-IPC interactions based on the composite representations.

290. A non-transitory computer-readable medium for system for predicting an amino acid-immunoprotein complex (IPC) interaction comprising instructions that, when executed by one or more processors of one or more computing devices, cause the one or more processors to:
access a set of amino acid sequences, each of the amino acid sequences of the set having been identified from at least one protein;
access an immunoprotein complex (IPC) sequence identified for an IPC of a subject;
process, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
process, using a second processing block in the processing subsystem, an IPC sequence representation to generate a transformed IPC sequence representation, wherein the IPC sequence representation was generated based on the identified IPC sequence appended with a BOS token, and wherein the set of amino acid sequence representations and the IPC sequence representation are processed in parallel;
generate composite representations by combining each of the transformed BOS token representations of the set of transformed amino acid sequence representations with the transformed BOS token representation of the transformed IPC sequence representation; and
determine one or more predicted amino acid-IPC interactions based on the composite representations.

291. A vaccine comprising:
one or more peptides;
a plurality of nucleic acids that encode the one or more peptides; or
a plurality of cells expressing the one or more peptides,
wherein the one or more peptides are selected from among a set of peptides by:
   accessing a set of amino acid sequences, each of the amino acid sequences of the set having been identified from at least one protein;
   accessing an immunoprotein complex (IPC) sequence identified for an IPC of a subject;
   processing, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
   processing, using a second processing block in the processing subsystem, an IPC sequence representation to generate a transformed IPC sequence representation, wherein the IPC sequence representation was generated based on the identified IPC sequence appended with a BOS token, and wherein the set of amino acid sequence representations and the IPC sequence representation are processed in parallel;
   generating composite representations by combining each of the transformed BOS token representations of the set of transformed amino acid sequence representations with the transformed BOS token representation of the transformed IPC sequence representation; and
   determining one or more predicted amino acid-IPC interactions based on the composite representations.

292. A method for manufacturing a vaccine comprising:
producing a vaccine comprising:
one or more peptides;
a plurality of nucleic acids that encode the one or more peptides; or
a plurality of cells expressing the one or more peptides,
wherein the one or more peptides are selected from among a set of peptides by:
   accessing a set of amino acid sequences, each of the amino acid sequences of the set having been identified from at least one protein;
   accessing an immunoprotein complex (IPC) sequence identified for an IPC of a subject;
   processing, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
   processing, using a second processing block in the processing subsystem, an IPC sequence representation to generate a transformed IPC sequence representation, wherein the IPC sequence representation was generated based on the identified IPC sequence appended with a BOS token, and wherein the set of amino acid sequence representations and the IPC sequence representation are processed in parallel;
   generating composite representations by combining each of the transformed BOS token representations of the set of transformed amino acid sequence representations with the transformed BOS token representation of the transformed IPC sequence representation; and
   determining one or more predicted amino acid-IPC interactions based on the composite representations.

293. A pharmaceutical composition comprising one or more peptides selected from among a set of peptides by:
accessing a set of amino acid sequences, each of the amino acid sequences of the set having been identified from at least one protein;
accessing an immunoprotein complex (IPC) sequence identified for an IPC of a subject;
processing, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
processing, using a second processing block in the processing subsystem, an IPC sequence representation to generate a transformed IPC sequence representation, wherein the IPC sequence representation was generated based on the identified IPC sequence appended with a BOS token, and wherein the set of amino acid sequence representations and the IPC sequence representation are processed in parallel;
generating composite representations by combining each of the transformed BOS token representations of the set of transformed amino acid sequence representations with the transformed BOS token representation of the transformed IPC sequence representation; and
determining one or more predicted amino acid-IPC interactions based on the composite representations.

294. A computer-implemented method for predicting an amino acid-immunoprotein complex (IPC) interaction comprising:

accessing a set of amino acid sequences, each of the amino acid sequences of the set having been identified from at least one protein;
accessing an immunoprotein complex (IPC) sequence identified for an IPC of a subject;
processing, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
processing, using a second processing block in the processing subsystem, the IPC sequence to generate an IPC sequence embedding;
generating composite representations by aggregating each of the transformed BOS token representations of the set of transformed amino acid sequence representations with the IPC sequence embedding; and
determining one or more predicted amino acid-IPC interactions based on the composite representations.

295. The computer-implemented method of embodiment 294, wherein the IPC of the subject is a major histocompatibility complex (MHC) comprising MHC Class I (MHC-I) and/or MHC Class II (MHC-II) or is a T-cell receptor (TCR), and wherein the at least one protein is a therapeutic protein or is present in a disease sample from the subject.

296. The computer-implemented method of embodiment 294, wherein processing a set of amino acid sequence representations comprises processing a peptide beginning-of-sequence (BOS) representation to generate a transformed peptide sequence representation.

297. The computer-implemented method of embodiment 294, wherein processing the set of amino acid sequence representations comprises:
transforming the set of amino acid sequence representations using the one or more first processing blocks into the set of transformed amino acid sequence representations, wherein each of the one or more first processing blocks includes a set of processing sub-blocks.

298. The computer-implemented method of embodiment 294, wherein the machine-learning model includes one or more transformer encoders, each of the one or more transformer encoders including a processing layer.

299. The computer-implemented method of embodiment 294, wherein the set of amino acid sequence representations comprises aggregate sequence representations, the aggregate sequence representations including a set of peptide representations and one or more of: a set of amino-terminal flanking (N-flank) representations or a set of carboxy-terminal flanking (C-flank) representations.

300. The computer-implemented method of embodiment 294, further comprising, prior to generating the set of transformed amino acid sequence representations:
flattening the aggregate sequence representations into a single array; and
densifying the aggregate sequence representations by removing empty rows from the array, wherein the transformed amino acid sequence representations are generated based on the densified aggregate sequence representations.

301. The computer-implemented method of embodiment 294, wherein processing the set of amino acid sequence representations comprises:
for each amino acid sequence representation of the set:
   determining, for each element of the amino acid sequence representation, a plurality of vectors based on a set of weights associated with a processing layer of the machine-learning model; and
   generating the set of element-focused scores based on the plurality of vectors and the set of weights.

302. The computer-implemented method of embodiment 294, wherein the one or more first processing blocks and the second processing block comprise attention blocks, each attention block comprising a set of attention sub-blocks, each attention sub-block comprising a self-attention layer; and
wherein the machine-learning model is an attention-based machine learning model, and
wherein the method further comprises: by one or more of the attention blocks, generating attention maps including one or more masks limiting attention applied by the attention sub-blocks to a sequence length according to the masks.

303. The computer-implemented method of embodiment 302, the method further comprising:
calculating a plurality of average attention values corresponding to a plurality of peptide positions by calculating, based on a set of peptides with a uniform length distribution, an average attention value at each peptide position of the plurality of peptide positions; and
subtracting the plurality of average attention values from a mask of the one or more masks.

304. The computer-implemented method of embodiment 294, further comprising obtaining a dataset for training the machine-learning model by:
generating, for a plurality of training peptides, a plurality of transformed peptide representations;
obtaining, for each training peptide, a corresponding cluster of training peptides based on the plurality of transformed peptide representations;
calculating, for each training peptide, an information content based on the corresponding cluster of training peptides; and
excluding one or more training peptides from the training data based on corresponding information contents of the one or more training peptides.

305. The computer-implemented method of embodiment 294, wherein the set of amino acid sequences comprises a peptide sequence and the IPC sequence comprises a major histocompatibility complex (MHC) sequence, and
wherein the one or more predicted amino acid-IPC interactions comprise one or more of:
   an interaction affinity prediction for a peptide-IPC combination that predicts a binding affinity between a peptide and MHC;
   an interaction prediction for the peptide-IPC combination that predicts whether the MHC will present the peptide at a cell surface; or
   an immunogenicity prediction for the peptide-IPC combination that predicts an ability of the peptide to provoke an immune response with respect to the MHC.

306. The computer-implemented method of embodiment 294, wherein determining the one or more predicted amino acid-IPC interactions comprises:
processing the composite representations to generate a set of results; and
selecting an amino acid-IPC combination based on a highest result among the set of results.

307. The computer-implemented method of embodiment 294, wherein the one or more predicted amino acid-IPC interactions comprise a prediction of tumor-specific immunogenicity of a peptide.

308. The computer-implemented method of embodiment 294, wherein the set of amino acid sequences comprises a set of peptide sequences, wherein the one or more predicted amino acid-IPC interactions identify a subset of peptide sequences having increased tumor-specific immunogenicity or increased likelihood of presentation by the IPC relative to the set of peptide sequences.

309. The computer-implemented method of embodiment 294, further comprising:
identifying a subset of peptides from the set of amino acid sequences to include in an individualized vaccine, to include as a target for an immunotherapy, and/or to exclude as a target for an immunotherapy based on the determined one or more predicted amino acid-IPC interactions.

310. The computer-implemented method of embodiment 294, further comprising:
accessing a protein sequence corresponding to the at least one protein;
obtaining a protein sequence embedding based on the protein sequence; and
determining the one or more predicted amino acid-IPC interactions based at least partially on the protein sequence embedding.

311. The computer-implemented method of embodiment 294, wherein the protein language model comprises a pretrained protein language model.

312. The computer-implemented method of embodiment 310, further comprising:
reducing a dimensionality of the protein sequence embedding; and
combining each of the transformed BOS token representations of the set of transformed amino acid sequence representations with the transformed BOS token representation of the transformed IPC sequence representation and the dimensionality-reduced protein sequence embedding.

313. The computer-implemented method of embodiment 312, wherein the dimensionality of the protein sequence embedding is reduced via a neural network.

314. The computer-implemented method of embodiment 294, wherein generating the IPC sequence embedding comprises: inputting the IPC sequence into a protein language model.

315. The computer-implemented method of embodiment 294, further comprising:
reducing a dimensionality of the IPC sequence embedding.

316. The computer-implemented method of embodiment 315, wherein the dimensionality of the IPC sequence embedding is reduced via Principal Component Analysis (PCA).

317. The computer-implemented method of embodiment 315, wherein generating the composite representations comprises: for each of the set of transformed amino acid sequence representations, elementwise multiplying a transformed amino acid beginning-of-sequence (BOS) representation corresponding to the transformed amino acid sequence representation by the dimensionality reduced IPC sequence embedding.

318. A system for predicting an amino acid-immunoprotein complex (IPC) interaction, comprising:
one or more non-transitory computer-readable storage media including instructions; and
one or more processors coupled to the one or more storage media, the one or more processors configured to execute the instructions to: accessing a set of amino acid sequences, each of the amino acid sequences of the set having been identified from at least one protein;
   access a set of amino acid sequences, each of the amino acid sequences of the set having been identified from at least one protein;
   access an immunoprotein complex (IPC) sequence identified for an IPC of a subject;
   process, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
   process, using a second processing block in the processing subsystem, the IPC sequence to generate an IPC sequence embedding;
   generate composite representations by aggregating each of the transformed BOS token representations of the set of transformed amino acid sequence representations with the IPC sequence embedding; and
   determine one or more predicted amino acid-IPC interactions based on the composite representations.

319. A non-transitory computer-readable medium for system for predicting an amino acid-immunoprotein complex (IPC) interaction comprising instructions that, when executed by one or more processors of one or more computing devices, cause the one or more processors to:
access a set of amino acid sequences, each of the amino acid sequences of the set having been identified from at least one protein;
access an immunoprotein complex (IPC) sequence identified for an IPC of a subject;
process, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
process, using a second processing block in the processing subsystem, the IPC sequence to generate an IPC sequence embedding;
generate composite representations by aggregating each of the transformed BOS token representations of the set of transformed amino acid sequence representations with the IPC sequence embedding; and
determine one or more predicted amino acid-IPC interactions based on the composite representations.

320. A vaccine comprising:
one or more peptides;
a plurality of nucleic acids that encode the one or more peptides; or
a plurality of cells expressing the one or more peptides,
wherein the one or more peptides are selected from among a set of peptides by:
   accessing a set of amino acid sequences, each of the amino acid sequences of the set having been identified from at least one protein;
   accessing an immunoprotein complex (IPC) sequence identified for an IPC of a subject;
   processing, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
   processing, using a second processing block in the processing subsystem, the IPC sequence to generate an IPC sequence embedding;
   generating composite representations by aggregating each of the transformed BOS token representations of the set of transformed amino acid sequence representations with the IPC sequence embedding; and
   determining one or more predicted amino acid-IPC interactions based on the composite representations.

321. A method for manufacturing a vaccine comprising:
producing a vaccine comprising:
one or more peptides;
a plurality of nucleic acids that encode the one or more peptides; or
a plurality of cells expressing the one or more peptides,
wherein the one or more peptides are selected from among a set of peptides by:
   accessing a set of amino acid sequences, each of the amino acid sequences of the set having been identified from at least one protein;
   accessing an immunoprotein complex (IPC) sequence identified for an IPC of a subject;
   processing, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
   processing, using a second processing block in the processing subsystem, the IPC sequence to generate an IPC sequence embedding;
   generating composite representations by aggregating each of the transformed BOS token representations of the set of transformed amino acid sequence representations with the IPC sequence embedding; and
   determining one or more predicted amino acid-IPC interactions based on the composite representations.

322. A pharmaceutical composition comprising one or more peptides selected from among a set of peptides by:
accessing a set of amino acid sequences, each of the amino acid sequences of the set having been identified from at least one protein;
accessing an immunoprotein complex (IPC) sequence identified for an IPC of a subject;
processing, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
processing, using a second processing block in the processing subsystem, the IPC sequence to generate an IPC sequence embedding;
generating composite representations by aggregating each of the transformed BOS token representations of the set of transformed amino acid sequence representations with the IPC sequence embedding; and
determining one or more predicted amino acid-IPC interactions based on the composite representations.

323. A computer-implemented method for predicting an amino acid-immunoprotein complex (IPC) interaction comprising:
accessing a set of amino acid sequences, each of the amino acid sequences of the set having been identified from at least one protein;
accessing an immunoprotein complex (IPC) sequence identified for an IPC of a subject;
generating an IPC sequence embedding based on the IPC sequence;
processing, using one or more processing blocks in a processing subsystem of a machine-learning model, the set of amino acid sequences to generate a set of transformed amino acid sequence representations based on a set of element-focused scores representing binding cores of the set of amino acid sequence representations;
generating, using a cross-attention machine-learning module in the processing subsystem, a set of composite representations based on the set of transformed amino acid sequence representations and the IPC sequence embedding; and
determining one or more predicted amino acid-IPC interactions based on the composite representations.

324. The computer-implemented method of embodiment 323, wherein generating the IPC sequence embedding comprises: inputting the IPC sequence into a protein language model.

325. The computer-implemented method of embodiment 323, further comprising:
reducing a dimensionality of the IPC sequence embedding.

326. The computer-implemented method of embodiment 325, wherein the dimensionality of the IPC sequence embedding is reduced via Principal Component Analysis (PCA).

327. The computer-implemented method of embodiment 325, wherein the cross-attention module comprises a self-attention transformer having three components: a Query (Q) component, a Key (K) component, and a Value (V) component.

328. The computer-implemented method of embodiment 327, wherein:
each of the K component and the V component corresponds to the set of transformed amino acid sequence representations; and
the Q component corresponds to an aggregation of a beginning-of-sequence (BOS) vector embedding and the dimensionality reduced IPC sequence embedding.

329. The computer-implemented method of embodiment 327, wherein:
each of the K component and the V component corresponds to the set of transformed amino acid sequence representations; and
the Q component corresponds to the dimensionality reduced IPC sequence embedding.

330. The computer-implemented method of embodiment 323, wherein:
wherein the set of amino acid sequences comprises at least one peptide sequence having a plurality of binding cores that can be bound to a plurality of alleles of the IPC, and
wherein the one or more predicted amino acid-IPC interactions comprise at least a plurality of allele-specific and binding-core-specific predicted amino acid-IPC interactions.

331. The computer-implemented method of embodiment 323, wherein the IPC of the subject is a major histocompatibility complex (MHC) comprising MHC Class I (MHC-I) and/or MHC Class II (MHC-II) or is a T-cell receptor (TCR), and wherein the at least one protein is a therapeutic protein or is present in a disease sample from the subject.

332. The computer-implemented method of embodiment 323, wherein processing the set of amino acid sequence representations comprises: transforming the set of amino acid sequence representations using the one or more first processing blocks into the set of transformed amino acid sequence representations, wherein each of the one or more first processing blocks includes a set of processing sub-blocks.

333. The computer-implemented method of embodiment 323, wherein the set of amino acid sequence representations comprises aggregate sequence representations, the aggregate sequence representations including a set of peptide representations and one or more of: a set of amino-terminal flanking (N-flank) representations or a set of carboxy-terminal flanking (C-flank) representations.

334. The computer-implemented method of embodiment 323, wherein processing the set of amino acid sequence representations comprises:
for each amino acid sequence representation of the set:
   determining, for each element of the amino acid sequence representation, a plurality of vectors based on a set of weights associated with a processing layer of the machine-learning model; and
   generating the set of element-focused scores based on the plurality of vectors and the set of weights.

335. The computer-implemented method of embodiment 323, wherein the one or more processing blocks comprise attention blocks, each attention block comprising a set of attention sub-blocks, each attention sub-block comprising a self-attention layer; and
wherein the machine-learning model is an attention-based machine learning model, and
wherein the method further comprises: by one or more of the attention blocks, generating attention maps including one or more masks limiting attention applied by the attention sub-blocks to a sequence length according to the masks.

336. The computer-implemented method of embodiment 335, the method further comprising:
calculating a plurality of average attention values corresponding to a plurality of peptide positions by calculating, based on a set of peptides with a uniform length distribution, an average attention value at each peptide position of the plurality of peptide positions; and
subtracting the plurality of average attention values from a mask of the one or more masks.

337. The computer-implemented method of embodiment 323, further comprising obtaining a dataset for training the machine-learning model by:
generating, for a plurality of training peptides, a plurality of transformed peptide representations;
obtaining, for each training peptide, a corresponding cluster of training peptides based on the plurality of transformed peptide representations;
calculating, for each training peptide, an information content based on the corresponding cluster of training peptides; and
excluding one or more training peptides from the training data based on corresponding information contents of the one or more training peptides.

338. The computer-implemented method of embodiment 323, wherein the set of amino acid sequences comprises a peptide sequence and the IPC sequence comprises a major histocompatibility complex (MHC) sequence, and
wherein the one or more predicted amino acid-IPC interactions comprise one or more of:
   an interaction affinity prediction for a peptide-IPC combination that predicts a binding affinity between a peptide and MHC;
   an interaction prediction for the peptide-IPC combination that predicts whether the MHC will present the peptide at a cell surface; or
   an immunogenicity prediction for the peptide-IPC combination that predicts an ability of the peptide to provoke an immune response with respect to the MHC.

339. The computer-implemented method of embodiment 323, wherein determining the one or more predicted amino acid-IPC interactions comprises:
processing the composite representations to generate a set of results; and
selecting an amino acid-IPC combination based on a highest result among the set of results.

340. The computer-implemented method of embodiment 323, wherein the one or more predicted amino acid-IPC interactions comprise a prediction of tumor-specific immunogenicity of a peptide.

341. The computer-implemented method of embodiment 323, wherein the set of amino acid sequences comprises a set of peptide sequences, wherein the one or more predicted amino acid-IPC interactions identify a subset of peptide sequences having increased tumor-specific immunogenicity or increased likelihood of presentation by the IPC relative to the set of peptide sequences.

342. The computer-implemented method of embodiment 323, further comprising:
identifying a subset of peptides from the set of amino acid sequences to include in an individualized vaccine, to include as a target for an immunotherapy, and/or to exclude as a target for an immunotherapy based on the determined one or more predicted amino acid-IPC interactions.

343. The computer-implemented method of embodiment 323, further comprising:
accessing a protein sequence corresponding to the at least one protein;
obtaining a protein sequence embedding based on the protein sequence and a protein language model; and
determining the one or more predicted amino acid-IPC interactions based at least partially on the protein sequence embedding.

344. The computer-implemented method of embodiment 343, wherein the protein language model comprises a pretrained protein language model.

345. The computer-implemented method of embodiment 344, wherein the composite representations are generated based at least partially on the protein sequence embedding.

346. A system for predicting an amino acid-immunoprotein complex (IPC) interaction, comprising:
one or more non-transitory computer-readable storage media including instructions; and
one or more processors coupled to the one or more storage media, the one or more processors configured to execute the instructions to:
   access a set of amino acid sequences, each of the amino acid sequences of the set having been identified from at least one protein;
   access an immunoprotein complex (IPC) sequence identified for an IPC of a subject;
   generate an IPC sequence embedding based on the IPC sequence;
   process, using one or more processing blocks in a processing subsystem of a machine-learning model, the set of amino acid sequences to generate a set of transformed amino acid sequence representations based on a set of element-focused scores representing binding cores of the set of amino acid sequence representations;
   generate, using a cross-attention machine-learning module in the processing subsystem, a set of composite representations based on the set of transformed amino acid sequence representations and the IPC sequence embedding; and
   determine one or more predicted amino acid-IPC interactions based on the composite representations.

347. A non-transitory computer-readable medium for system for predicting an amino acid-immunoprotein complex (IPC) interaction comprising instructions that, when executed by one or more processors of one or more computing devices, cause the one or more processors to:
access a set of amino acid sequences, each of the amino acid sequences of the set having been identified from at least one protein;
access an immunoprotein complex (IPC) sequence identified for an IPC of a subject;
generate an IPC sequence embedding based on the IPC sequence;
process, using one or more processing blocks in a processing subsystem of a machine-learning model, the set of amino acid sequences to generate a set of transformed amino acid sequence representations based on a set of element-focused scores representing binding cores of the set of amino acid sequence representations;
generate, using a cross-attention machine-learning module in the processing subsystem, a set of composite representations based on the set of transformed amino acid sequence representations and the IPC sequence embedding; and
determine one or more predicted amino acid-IPC interactions based on the composite representations.

348. A vaccine comprising:
one or more peptides;
a plurality of nucleic acids that encode the one or more peptides; or
a plurality of cells expressing the one or more peptides,
wherein the one or more peptides are selected from among a set of peptides by:
   accessing a set of amino acid sequences, each of the amino acid sequences of the set having been identified from at least one protein;
   accessing an immunoprotein complex (IPC) sequence identified for an IPC of a subject;
   generating an IPC sequence embedding based on the IPC sequence;
   processing, using one or more processing blocks in a processing subsystem of a machine-learning model, the set of amino acid sequences to generate a set of transformed amino acid sequence representations based on a set of element-focused scores representing binding cores of the set of amino acid sequence representations;
   generating, using a cross-attention machine-learning module in the processing subsystem, a set of composite representations based on the set of transformed amino acid sequence representations and the IPC sequence embedding; and
   determining one or more predicted amino acid-IPC interactions based on the composite representations.

349. A method for manufacturing a vaccine comprising:
producing a vaccine comprising:
one or more peptides;
a plurality of nucleic acids that encode the one or more peptides; or
a plurality of cells expressing the one or more peptides,
wherein the one or more peptides are selected from among a set of peptides by:
   accessing a set of amino acid sequences, each of the amino acid sequences of the set having been identified from at least one protein;
   accessing an immunoprotein complex (IPC) sequence identified for an IPC of a subject;
   generating an IPC sequence embedding based on the IPC sequence;
   processing, using one or more processing blocks in a processing subsystem of a machine-learning model, the set of amino acid sequences to generate a set of transformed amino acid sequence representations based on a set of element-focused scores representing binding cores of the set of amino acid sequence representations;
   generating, using a cross-attention machine-learning module in the processing subsystem, a set of composite representations based on the set of transformed amino acid sequence representations and the IPC sequence embedding; and
   determining one or more predicted amino acid-IPC interactions based on the composite representations.

350. A pharmaceutical composition comprising one or more peptides selected from among a set of peptides by:
accessing a set of amino acid sequences, each of the amino acid sequences of the set having been identified from at least one protein;
accessing an immunoprotein complex (IPC) sequence identified for an IPC of a subject;
generating an IPC sequence embedding based on the IPC sequence;
processing, using one or more processing blocks in a processing subsystem of a machine-learning model, the set of amino acid sequences to generate a set of transformed amino acid sequence representations based on a set of element-focused scores representing binding cores of the set of amino acid sequence representations;
generating, using a cross-attention machine-learning module in the processing subsystem, a set of composite representations based on the set of transformed amino acid sequence representations and the IPC sequence embedding; and
determining one or more predicted amino acid-IPC interactions based on the composite representations.

The description provides preferred example embodiments only, and is not intended to limit the scope, applicability or configuration of the disclosure. Rather, the description of the preferred example embodiments will provide those skilled in the art with an enabling description for implementing various embodiments. It is understood that various changes can be made in the function and arrangement of elements without departing from the spirit and scope as set forth in the appended claims.

Specific details are given in the following description to provide a thorough understanding of the embodiments. However, it will be understood that the embodiments can be practiced without these specific details. For example, circuits, systems, networks, processes, and other components can be shown as components in block diagram form in order not to obscure the embodiments in unnecessary detail. In other instances, well-known circuits, processes, algorithms, structures, and techniques can be shown without unnecessary detail in order to avoid obscuring the embodiments.

What is claimed is:

1. A computer-implemented method for predicting an amino acid-immunoprotein complex (IPC) interaction comprising:
accessing a set of amino acid sequences, each of the amino acid sequences of the set having been identified from at least one protein;
accessing an immunoprotein complex (IPC) sequence identified for an IPC of a subject;
processing, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
processing, using a second processing block in the processing subsystem, an IPC sequence representation to generate a transformed IPC sequence representation, wherein the IPC sequence representation was generated based on the identified IPC sequence appended with a BOS token, and wherein the set of amino acid sequence representations and the IPC sequence representation are processed in parallel;
generating composite representations by combining each of the transformed BOS token representations of the set of transformed amino acid sequence representations with the transformed BOS token representation of the transformed IPC sequence representation; and
determining one or more predicted amino acid-IPC interactions based on the composite representations.

2. The computer-implemented method of claim 1, wherein the set of transformed amino acid sequence representations comprises a set of amino-terminal flanking (N-flank) representations or a set of carboxy-terminal flanking (C-flank) representations.

3. The computer-implemented method of claim 1, wherein the IPC of the subject is a major histocompatibility complex (MHC) comprising MHC Class I (MHC-I) and/or MHC Class II (MHC-II) or is a T-cell receptor (TCR), and wherein the at least one protein is a therapeutic protein or is present in a disease sample from the subject.

4. The computer-implemented method of claim 1, wherein generating composite representations comprises:
for each of the set of transformed amino acid sequence representations, elementwise multiplying a transformed amino acid beginning-of-sequence (BOS) representation corresponding to the transformed amino acid sequence representation by a transformed IPC beginning-of-sequence (BOS) representation corresponding to the transformed IPC sequence representation.

5. The computer-implemented method of claim 1, wherein processing a set of amino acid sequence representations comprises processing a peptide beginning-of-sequence (BOS) representation to generate a transformed peptide sequence representation, and wherein processing an IPC sequence representation comprises processing an MHC beginning-of-sequence representation (BOS) to generate a transformed MHC sequence representation.

6. The computer-implemented method of claim 1, wherein processing the set of amino acid sequence representations comprises:
for each amino acid sequence representation of the set:
determining, for each element of the amino acid sequence representation, a plurality of vectors based on a set of weights associated with a processing layer of the machine-learning model; and
generating the set of element-focused scores based on the plurality of vectors and the set of weights.

7. The computer-implemented method of claim 1, wherein the one or more first processing blocks and the second processing block comprise attention blocks, each attention block comprising a set of attention sub-blocks, each attention sub-block comprising a self-attention layer; and
wherein the machine-learning model is an attention-based machine learning model, and
wherein the method further comprises: by one or more of the attention blocks, generating attention maps including one or more masks limiting attention applied by the attention sub-blocks to a sequence length according to the masks.

8. The computer-implemented method of claim 1, wherein the set of amino acid sequences comprises a peptide sequence and the IPC sequence comprises a major histocompatibility complex (MHC) sequence, and
wherein the one or more predicted amino acid-IPC interactions comprise one or more of:
an interaction affinity prediction for a peptide-IPC combination that predicts a binding affinity between a peptide and MHC;
an interaction prediction for the peptide-IPC combination that predicts whether the MHC will present the peptide at a cell surface; or
an immunogenicity prediction for the peptide-IPC combination that predicts an ability of the peptide to provoke an immune response with respect to the MHC.

9. The computer-implemented method of claim 1, wherein the one or more predicted amino acid-IPC interactions comprise a prediction of tumor-specific immunogenicity of a peptide.

10. The computer-implemented method of claim 1, wherein the set of amino acid sequences comprises a set of peptide sequences, wherein the one or more predicted amino acid-IPC interactions identify a subset of peptide sequences having increased tumor-specific immunogenicity or increased likelihood of presentation by the IPC relative to the set of peptide sequences.

11. The computer-implemented method of claim 1, further comprising:
identifying a subset of peptides from the set of amino acid sequences to include in an individualized vaccine, to include as a target for an immunotherapy, and/or to exclude as a target for an immunotherapy based on the determined one or more predicted amino acid-IPC interactions.

12. A computer-implemented method for predicting an amino acid-immunoprotein complex (IPC) interaction comprising:
accessing a set of amino acid sequences, each of the amino acid sequences of the set having been identified from at least one protein;
accessing an immunoprotein complex (IPC) sequence identified for an IPC of a subject;
processing, using one or more first processing blocks in a processing subsystem of a machine-learning model, a set of amino acid sequence representations to generate a set of transformed amino acid sequence representations based on a set of element-focused scores representing binding cores of the set of amino acid sequence representations, wherein each of the amino acid sequence representations was generated based on one of the amino acid sequences appended with a beginning-of-sequence (BOS) token;
processing, using a second processing block in the processing subsystem, the IPC sequence to generate an IPC sequence embedding;
generating composite representations by aggregating each of the transformed BOS token representations of the set of transformed amino acid sequence representations with the IPC sequence embedding; and
determining one or more predicted amino acid-IPC interactions based on the composite representations.

13. The computer-implemented method of claim 12, wherein the IPC of the subject is a major histocompatibility complex (MHC) comprising MHC Class I (MHC-I) and/or MHC Class II (MHC-II) or is a T-cell receptor (TCR), and wherein the at least one protein is a therapeutic protein or is present in a disease sample from the subject.

14. The computer-implemented method of claim 12, wherein processing a set of amino acid sequence representations comprises processing a peptide beginning-of-sequence (BOS) representation to generate a transformed peptide sequence representation.

15. The computer-implemented method of claim 12, further comprising:
identifying a subset of peptides from the set of amino acid sequences to include in an individualized vaccine, to include as a target for an immunotherapy, and/or to exclude as a target for an immunotherapy based on the determined one or more predicted amino acid-IPC interactions.

16. The computer-implemented method of claim 12, further comprising:
accessing a protein sequence corresponding to the at least one protein;
obtaining a protein sequence embedding based on the protein sequence; and
determining the one or more predicted amino acid-IPC interactions based at least partially on the protein sequence embedding.

17. A computer-implemented method for predicting an amino acid-immunoprotein complex (IPC) interaction comprising:
accessing a set of amino acid sequences, each of the amino acid sequences of the set having been identified from at least one protein;
accessing an immunoprotein complex (IPC) sequence identified for an IPC of a subject;
generating an IPC sequence embedding based on the IPC sequence;
processing, using one or more processing blocks in a processing subsystem of a machine-learning model, the set of amino acid sequences to generate a set of transformed amino acid sequence representations based on a set of element-focused scores representing binding cores of the set of amino acid sequence representations;
generating, using a cross-attention machine-learning module in the processing subsystem, a set of composite representations based on the set of transformed amino acid sequence representations and the IPC sequence embedding; and
determining one or more predicted amino acid-IPC interactions based on the composite representations.

18. The computer-implemented method of claim 17, wherein:
the set of amino acid sequences comprises at least one peptide sequence having a plurality of binding cores that can be bound to a plurality of alleles of the IPC, and
the one or more predicted amino acid-IPC interactions comprise at least a plurality of allele-specific and binding-core-specific predicted amino acid-IPC interactions.

19. The computer-implemented method of claim 17, wherein the IPC of the subject is a major histocompatibility complex (MHC) comprising MHC Class I (MHC-I) and/or MHC Class II (MHC-II) or is a T-cell receptor (TCR), and wherein the at least one protein is a therapeutic protein or is present in a disease sample from the subject.

20. The computer-implemented method of claim 17, further comprising:
identifying a subset of peptides from the set of amino acid sequences to include in an individualized vaccine, to include as a target for an immunotherapy, and/or to exclude as a target for an immunotherapy based on the determined one or more predicted amino acid-IPC interactions.


SEQUENCE LISTING (78 sequences)

SEQ ID NO: 1 - protein, 10 aa, synthetic construct
GGSGGGGSGG

SEQ ID NO: 2 - RNA, 30 nt, synthetic construct
  source 1..30: mol_type = other RNA
ggcggctctggaggaggcggctccggaggc

SEQ ID NO: 3 - RNA, 39 nt, synthetic construct
  source 1..39: mol_type = other RNA
ttcttctggtccccacagactcagagagaacccgccacc

SEQ ID NO: 4 - RNA, 51 nt, synthetic construct
  source 1..51: mol_type = other RNA
ggcgaactagtattcttctggtccccacagactcagagagaacccgccacc

SEQ ID NO: 5 - protein, 26 aa, synthetic construct
MRVMAPRTLILLLSGALALTETWAGS

SEQ ID NO: 6 - RNA, 78 nt, synthetic construct
  source 1..78: mol_type = other RNA
atgagagtgatggcccccagaaccctgatcctgctgctgtctggcgccctggccctgacagagacatgggccggaagc

SEQ ID NO: 7 - protein, 55 aa, synthetic construct
IVGIVAGLAVLAVVVIGAVVATVMCRRKSSGGKGGSYSQAASSDSAQGSDVSLTA

SEQ ID NO: 8 - RNA, 165 nt, synthetic construct
  source 1..165: mol_type = other RNA
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcc

SEQ ID NO: 9 - RNA, 136 nt, synthetic construct
  source 1..136: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 10 - RNA, 143 nt, synthetic construct
  source 1..143: mol_type = other RNA
caagcacgcagcaatgcagctcaaaacgcttagcctagccacacccccacgggaaacagcagtgattaacctttagcaataaacgaaagtttaactaagctatactaaccccagggttggtcaatttcgtgccagccacaccg

SEQ ID NO: 11 - RNA, 317 nt, synthetic construct
  source 1..317: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 12 - RNA, 129 nt, synthetic construct
  source 1..129: mol_type = other RNA
ggcgaactagtattcttctggtccccacagactcagagagaacccgccaccatgagagtgatggcccccagaaccctgatcctgctgctgtctggcgccctggccctgacagagacatgggccggaagc

SEQ ID NO: 13 - RNA, 488 nt, synthetic construct
  source 1..488: mol_type = other RNA
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgct

SEQ ID NO: 14 - protein, 24 aa, synthetic construct
ZKAVKPKAAKPKVVKPKKAAPKKK

SEQ ID NO: 15 - protein, 5 aa, synthetic construct
GVGTP

SEQ ID NO: 16 - protein, 13 aa, synthetic construct
AAAAAAKAAAKAA

SEQ ID NO: 17 - protein, 5 aa, synthetic construct
KYGAA

SEQ ID NO: 18 - protein, 5 aa, synthetic construct
GVGTP

SEQ ID NO: 19 - protein, 13 aa, synthetic construct
AAAAAAKAAAKAA

SEQ ID NO: 20 - protein, 5 aa, synthetic construct
KYGAA

SEQ ID NO: 21 - protein, 5 aa, synthetic construct
GAGPA

SEQ ID NO: 22 - protein, 14 aa, synthetic construct
AAAAAAKAAAKAAQ

SEQ ID NO: 23 - protein, 5 aa, synthetic construct
FGLVG

SEQ ID NO: 24 - protein, 5 aa, synthetic construct
GAGPA

SEQ ID NO: 25 - protein, 15 aa, synthetic construct
AAAAAAKAAAKAAQF

SEQ ID NO: 26 - protein, 5 aa, synthetic construct
GLVGA

SEQ ID NO: 27 - protein, 5 aa, synthetic construct
GAGPA

SEQ ID NO: 28 - protein, 16 aa, synthetic construct
AAAAAAKAAAKAAQFG

SEQ ID NO: 29 - protein, 5 aa, synthetic construct
LVGAA

SEQ ID NO: 30 - protein, 5 aa, synthetic construct
GENRF

SEQ ID NO: 31 - protein, 17 aa, synthetic construct
YYTAGSSSPTHAKSAHV

SEQ ID NO: 32 - protein, 5 aa, synthetic construct
GENRF

SEQ ID NO: 33 - protein, 17 aa, synthetic construct
YYTAGSSSPTHAKSAHV

SEQ ID NO: 34 - protein, 5 aa, synthetic construct
GENRF

SEQ ID NO: 35 - protein, 18 aa, synthetic construct
YYTAGSSSPTHAKSAHVF

SEQ ID NO: 36 - protein, 5 aa, synthetic construct
ETKKK

SEQ ID NO: 37 - protein, 16 aa, synthetic construct
YYVKTYKIDVSSNGED

SEQ ID NO: 38 - protein, 5 aa, synthetic construct
WITIK

SEQ ID NO: 39 - protein, 5 aa, synthetic construct
ETKKK

SEQ ID NO: 40 - protein, 18 aa, synthetic construct
YYVKTYKIDVSSNGEDWI

SEQ ID NO: 41 - protein, 5 aa, synthetic construct
TIKEG

SEQ ID NO: 42 - protein, 83 aa, synthetic construct
DIQMTQSPSSLSASVGDRVTITCRASQGISSALAWYQQKPGKAPKLLIYSASYRYTGVPSRFSGSGSGTDFTFTISSLQPEDI

SEQ ID NO: 43 - protein, 15 aa, synthetic construct
DIQMTQSPSSLSASV

SEQ ID NO: 44 - protein, 15 aa, synthetic construct
IQMTQSPSSLSASVG

SEQ ID NO: 45 - protein, 15 aa, synthetic construct
QMTQSPSSLSASVGD

SEQ ID NO: 46 - protein, 15 aa, synthetic construct
MTQSPSSLSASVGDR

SEQ ID NO: 47 - protein, 12 aa, synthetic construct
SLSASVGDRVTI

SEQ ID NO: 48 - protein, 13 aa, synthetic construct
SLSASVGDRVTIT

SEQ ID NO: 49 - protein, 14 aa, synthetic construct
SLSASVGDRVTITC

SEQ ID NO: 50 - protein, 15 aa, synthetic construct
SLSASVGDRVTITCR

SEQ ID NO: 51 - protein, 12 aa, synthetic construct
LSASVGDRVTIT

SEQ ID NO: 52 - protein, 13 aa, synthetic construct
LSASVGDRVTITC

SEQ ID NO: 53 - protein, 12 aa, synthetic construct
SLSASVGDRVTI

SEQ ID NO: 54 - protein, 13 aa, synthetic construct
SLSASVGDRVTIT

SEQ ID NO: 55 - protein, 14 aa, synthetic construct
SLSASVGDRVTITC

SEQ ID NO: 56 - protein, 15 aa, synthetic construct
SLSASVGDRVTITCR

SEQ ID NO: 57 - protein, 12 aa, synthetic construct
LSASVGDRVTIT

SEQ ID NO: 58 - protein, 13 aa, synthetic construct
LSASVGDRVTITC

SEQ ID NO: 59 - protein, 12 aa, synthetic construct
SLSASVGDRVTI

SEQ ID NO: 60 - protein, 13 aa, synthetic construct
SLSASVGDRVTIT

SEQ ID NO: 61 - protein, 12 aa, synthetic construct
LSASVGDRVTIT

SEQ ID NO: 62 - protein, 13 aa, synthetic construct
LSASVGDRVTITC

SEQ ID NO: 63 - protein, 12 aa, synthetic construct
SYRYTGVPSRFS

SEQ ID NO: 64 - protein, 14 aa, synthetic construct
SYRYTGVPSRFSGS

SEQ ID NO: 65 - protein, 16 aa, synthetic construct
SYRYTGVPSRFSGSGS

SEQ ID NO: 66 - protein, 13 aa, synthetic construct
SYRYTGVPSRFSG

SEQ ID NO: 67 - protein, 15 aa, synthetic construct
SYRYTGVPSRFSGSG

SEQ ID NO: 68 - protein, 17 aa, synthetic construct
SYRYTGVPSRFSGSGSG

SEQ ID NO: 69 - protein, 16 aa, synthetic construct
YRYTGVPSRFSGSGSG

SEQ ID NO: 70 - protein, 14 aa, synthetic construct
YRYTGVPSRFSGSG

SEQ ID NO: 71 - protein, 13 aa, synthetic construct
YRYTGVPSRFSGS

SEQ ID NO: 72 - protein, 12 aa, synthetic construct
SYRYTGVPSRFS

SEQ ID NO: 73 - protein, 14 aa, synthetic construct
SYRYTGVPSRFSGS

SEQ ID NO: 74 - protein, 13 aa, synthetic construct
SYRYTGVPSRFSG

SEQ ID NO: 75 - protein, 14 aa, synthetic construct
YRYTGVPSRFSGSG

SEQ ID NO: 76 - protein, 13 aa, synthetic construct
YRYTGVPSRFSGS

SEQ ID NO: 77 - protein, 9 aa, synthetic construct
YTGVPSRFS

SEQ ID NO: 78 - protein, 9 aa, synthetic construct
RYTGVPSRF